(19) United States
(12) Patent Application Publication
(10) Pub. No.: US 2024/0238211 A1
(43) Pub. Date: Jul. 18, 2024

(54) ISOQUINOLINE-STABILIZED LIPID NANOPARTICLE FORMULATIONS

(71) Applicants: James C. COX, Somerville, MA (US); ModernaTX, Inc., Cambridge, MA (US)

(72) Inventors: Mark Brader, Lexington, MA (US); Jessica Banks, Sudbury, MA (US); Marek Kloczewiak, Newton, MA (US); James C. Cox, Somerville, MA (US); Dipendra Gyawali, East Walpole, MA (US)

(73) Assignee: ModernaTX, Inc., Cambridge, MA (US)

(21) Appl. No.: 18/556,221

(22) PCT Filed: Apr. 22, 2022
(86) PCT No.: PCT/US2022/025967

§ 371 Date: Oct. 19, 2023

Related U.S. Application Data
(60) Provisional application No. 63/229,761, filed on Aug. 5, 2021.
(60) Provisional application No. 63/179,033, filed on Apr. 23, 2021.

(51) Int. Cl. A61K 9/51; A61K 9/00; A61K 9/127; A61K 39/00; A61K 39/39

(52) U.S. Cl. CPC A61K 9/5123; A61K 9/0019; A61K 9/127; A61K 39/39; A61K 2039/53; A61K 2039/54; A61K 2039/55555

(57) ABSTRACT

Stabilized formulations of lipids and nucleic acids, including lipid nanoparticle formulations which encapsulate nucleic acids. Methods of making and of use of the formulations stabilized, by chemical compounds are also provided.

### RELATED APPLICATIONS

This application claims priority under 35 U.S.C. § 119(e) to U.S. Provisional application, U.S. Ser. No. 63/179,033, filed Apr. 23, 2021, and to U.S. Provisional application, U.S. Ser. No. 63/229,761, filed on Aug. 5, 2021, each of which is incorporated herein by reference.

### FIELD

Provided are formulations of lipids and nucleic acids, including lipid nanoparticle formulations which encapsulate nucleic acids, and more specifically to formulations stabilized by chemical compounds.

### BACKGROUND

The use of messenger RNA as a pharmaceutical agent is of great interest for a variety of applications, including in therapeutics, vaccines and diagnostics. Effective in vivo delivery of mRNA formulations represents a continuing challenge, as many such formulations are inherently unstable, activate an immune response, are susceptible to degradation by nucleases, or fail to reach their target organs or cells within the body due to issues with biodistribution. Each of these challenges results in loss of translational potency and therefore hinders efficacy of conventional mRNA pharmaceutical agents.

Various non-viral delivery systems, including nanoparticle formulations, present attractive opportunities to overcome many challenges associated with mRNA delivery. In particular, lipid nanoparticles (LNPs) have drawn particular attention in recent years as various LNP formulations have shown promise in a variety of pharmaceutical applications.

However, lipids have been shown to degrade nucleic acids including mRNA, and lipid nanoparticle formulations undergo rapid loss of purity when stored as refrigerated liquids. It is also evident that the stability of mRNA is poorer when encapsulated within LNPs than when stored unencapsulated.

### SUMMARY

Provided, among other things, are compositions and methods for the stabilization of nucleic acids. Some aspects encompass the observation that the mixture of certain compounds with lipid nanoparticle formulations comprising nucleic acids and/or nucleic acid formulations resulted in substantially improved formulation stability.

According to some aspects, stabilized pharmaceutical compositions are provided herein. In some embodiments, a stabilized pharmaceutical composition comprises a nucleic acid formulation comprising a nucleic acid and a stabilizing compound (e.g., a compound of Formula I, of Formula II, or a tautomer or solvate thereof). In some embodiments, a stabilized pharmaceutical composition comprises a nucleic acid formulation comprising a nucleic acid and a lipid, and a compound of Formula I:

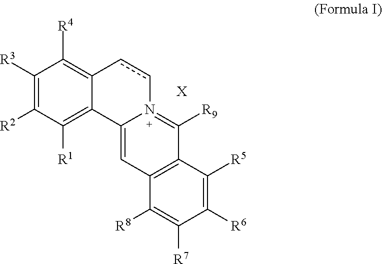

or a tautomer or solvate thereof, wherein:
[symbol] is a single bond or a double bond;
R¹ is H;
R² is OCH₃, or together with R³ is OCH₂O;
R³ is OCH₃, or together with R² is OCH₂O;
R⁴ is H;
R⁵ is H or OCH₃;
R⁶ is OCH₃;
R⁷ is H or OCH₃;
R⁸ is H;
R⁹ is H or CH₃; and
X is a pharmaceutically acceptable anion.

In some embodiments, a stabilized pharmaceutical composition comprises a nucleic acid formulation comprising a nucleic acid and a lipid, and a compound of Formula II:

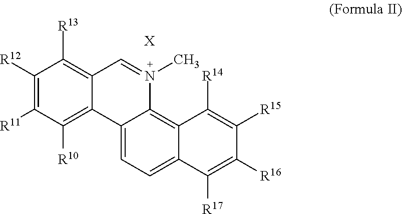

or a tautomer or solvate thereof, wherein:
R¹⁰ is H;
R¹¹ is H;
R¹² together with R¹³ is OCH₂O;
R¹⁴ is H;
R¹⁵ together with R¹⁶ is OCH₂O;
R¹⁷ is H; and
X is a pharmaceutically acceptable anion.

In some embodiments, the compound of Formula I has the structure of:

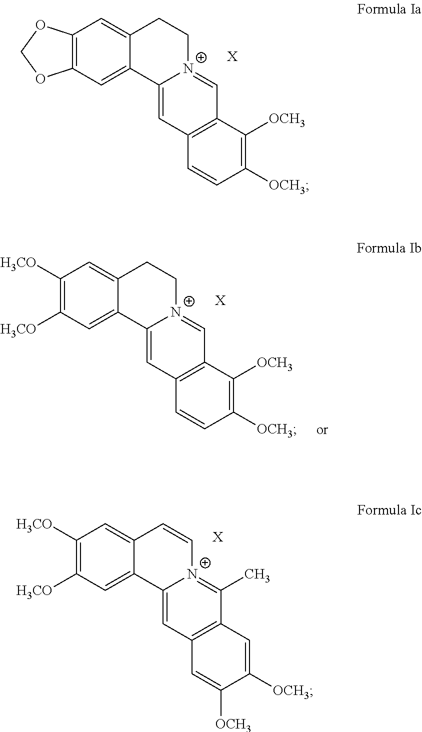

or a tautomer or solvate thereof.

In some embodiments, the compound of Formula II has the structure of:

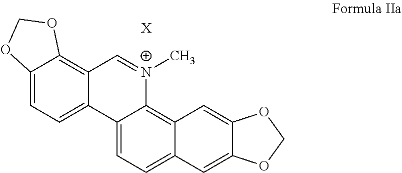

or a tautomer or solvate thereof.

In some embodiments, the nucleic acid formulation comprises lipid nanoparticles. In some embodiments, the nucleic acid formulation comprises liposomes. In some embodiments, the nucleic acid formulation comprises a lipoplex. In some embodiments, the nucleic acid is encapsulated within the lipid nanoparticles, liposomes, or lipoplex.

In some embodiments, the nucleic acid of the stabilized pharmaceutical composition is mRNA.

In some embodiments, the compound (i.e., the compound of Formula I or Formula II) has a purity of at least 70%, 80%, 90%, 95%, or 99%.

In some embodiments, the compound contains fewer than 100 ppm of elemental metals.

In some embodiments, a composition disclosed herein further comprises a pharmaceutically acceptable metal chelator. Exemplary, non-limiting, examples of such metal chelators are EDTA (ethylenediaminetetraacetic acid) and DTPA (diethylenetriaminepentaacetic acid).

In some embodiments, a composition disclosed herein is formulated in an aqueous solution.

In some embodiments, the aqueous solution comprises lipid nanoparticles and the nucleic acid is encapsulated in the lipid nanoparticles.

In some embodiments, the aqueous solution has a pH of or about 5 to 8, including pH of about 5, 5.5, 6, 6.5, 7, 7.5, or 8.

In some embodiments, the aqueous solution does not comprise NaCl. In some embodiments, the aqueous solution comprises NaCl in a concentration of or about 150 mM.

In some embodiments, the aqueous solution comprises a phosphate buffer, a tris buffer, an acetate buffer, a histidine buffer, or a citrate buffer.

In some embodiments, the compound is present at a concentration between about 0.1 mM and about 10 mM in an aqueous solution. In some embodiments, the compound is present at a concentration of or about 2 mM in an aqueous solution. In some embodiments, the compound is present at a concentration of or about 1 mM in an aqueous solution. In some embodiments, the compound is present at a concentration of or about 0.5 mM in an aqueous solution.

In some embodiments, the nucleic acid composition disclosed herein is a lyophilized product.

In some embodiments, the lyophilized product comprises lipid nanoparticles and the nucleic acid is encapsulated in the lipid nanoparticles.

According to some aspects, stabilized pharmaceutical compositions are provided herein, comprising a nucleic acid formulation comprising a nucleic acid and a lipid, and a compound of Formula Ia, or a tautomer or solvate thereof.

According to some aspects, stabilized pharmaceutical compositions are provided herein, comprising a nucleic acid formulation comprising a nucleic acid and a lipid, and a compound of Formula Ib, or a tautomer or solvate thereof.

According to some aspects, stabilized pharmaceutical compositions are provided herein, comprising a nucleic acid formulation comprising a nucleic acid and a lipid, and a compound of Formula Ic, or a tautomer or solvate thereof.

According to some aspects, stabilized pharmaceutical compositions are provided herein, comprising a nucleic acid formulation comprising a nucleic acid and a lipid, and a compound of Formula IIa, or a tautomer or solvate thereof.

In some embodiments, the nucleic acid formulation comprises lipid nanoparticles. In some embodiments, the nucleic acid formulation comprises liposomes. In some embodiments, the nucleic acid formulation comprises a lipoplex.

In some embodiments, the nucleic acid is encapsulated within the lipid nanoparticles, liposomes, or lipoplex. In some embodiments, the nucleic acid is mRNA.

In some embodiments, the compound of Formula I or Formula II has a purity of at least 70%, 80%, 90%, 95%, or 99%. In some embodiments, the compound of Formula I contains fewer than 100 ppm of elemental metals.

In some embodiments, the composition is formulated in an aqueous solution. In some embodiments, the aqueous solution comprises lipid nanoparticles and the nucleic acid is encapsulated in the lipid nanoparticles. In some embodiments, the aqueous solution has a pH of or about 5 to 8, including pH of about 5, 5.5, 6, 6.5, 7, 7.5, or 8. In some embodiments, the aqueous solution does not comprise NaCl. In some embodiments, the aqueous solution comprises NaCl in a concentration of or about 150 mM. In some embodiments, the aqueous solution comprises a buffer. In some embodiments, the aqueous solution comprises a phosphate buffer, a tris buffer, an acetate buffer, a histidine buffer, or a citrate buffer. In some embodiments, the concentration of the buffering agent(s) is about 2-10 mM.

In some embodiments, the compound of Formula I or Formula II is present at a concentration of less than about 10 mM. In some embodiments, the compound is present at a concentration between about 0.1 mM and about 10 mM. In some embodiments, the compound of Formula I is present at a concentration of or about 2 mM. In some embodiments, the compound of Formula I is present at a concentration of or about 1 mM. In some embodiments, the compound of Formula I is present at a concentration of or about 0.5 mM.

In some embodiments, the nucleic acid is a lyophilized product. In some embodiments, the lyophilized product comprises lipid nanoparticles and the nucleic acid is encapsulated in the lipid nanoparticles.

According to some aspects, compositions disclosed herein are used for the treatment of a disease in a subject. In some embodiments, the disease is caused by an infectious agent. In some embodiments, the disease is caused by or associated with a virus. In some embodiments, the disease is a disease caused by or associated with a malignant cell. In some embodiments, the disease is cancer.

According to some aspects, compositions having properties which inhibit microbial growth are disclosed herein. In some embodiments, microbial growth in a composition disclosed herein is inhibited by a compound disclosed herein. In some embodiments, a composition disclosed herein does not comprise phenol, m-cresol, or benzyl alcohol.

According to some aspects, methods of formulating nucleic acids are disclosed herein. In some embodiments, a method of formulating a nucleic acid comprises adding to a composition comprising a nucleic acid and a lipid, a compound of Formula I, or a tautomer or solvate thereof, to obtain a formulated composition. In certain embodiments, the compound of Formula I is of Formula Ia, Formula Ib, or Formula Ic, or a tautomer or solvate thereof.

In some embodiments, a method of formulating a nucleic acid comprises adding to a composition comprising a nucleic acid and a lipid, a compound of Formula II, or a tautomer or solvate thereof, to obtain a formulated composition. In certain embodiments, the compound of Formula II is of Formula IIa, or a tautomer or solvate thereof.

In some embodiments, the formulated composition comprises lipid nanoparticles. In some embodiments, the formulated composition further comprises liposomes. In some embodiments, the formulated composition further comprises a lipoplex. In some embodiments, the nucleic acid is encapsulated in the lipid nanoparticles, liposomes, or lipoplex.

In some embodiments, the method further comprises subsequently removing the compound of Formula I or the compound of Formula II from the formulated composition.

In some embodiments of the method of formulating a nucleic acid, the compound is Formula I, e.g., Formula Ia, Ib, Ic, or a tautomer or solvate thereof. In certain embodiments, the compound is Formula II, e.g., Formula IIa, or a tautomer or solvate thereof.

In some embodiments, the composition is a lyophilized product. In some embodiments, the lyophilized product comprises lipid nanoparticles. In some embodiments, the lipid nanoparticles encapsulate a nucleic acid.

According to some aspects, methods of processing mRNA-lipid nanoparticles are provided herein. In some embodiments, pharmaceutically acceptable methods of processing an mRNA-lipid nanoparticle for therapeutic injection are provided, comprising adding a compound of Formula I or Formula II, or a tautomer or solvate thereof, to a lipid nanoparticle, and subsequently adding an mRNA to the lipid nanoparticle-compound mixture. In some embodiments, pharmaceutically acceptable methods of conferring anti-microbial properties to an mRNA-lipid nanoparticle composition are provided, comprising adding a compound of Formula I or Formula II, or a tautomer or solvate thereof, to the mRNA-lipid nanoparticle composition. In some embodiments, a pharmaceutically acceptable method of processing an mRNA-lipid nanoparticle for therapeutic injection comprises adding an mRNA to a lipid nanoparticle, and subsequently adding a compound of Formula I or Formula II, or a tautomer or solvate thereof, to the lipid nanoparticle-mRNA mixture. In some embodiments, a pharmaceutically acceptable method of processing an mRNA-lipid nanoparticle for therapeutic injection comprises combining an mRNA, a lipid nanoparticle, and a compound of Formula I or Formula II, or a tautomer or solvate thereof.

According to some aspects, compositions of lipid nanoparticles and mRNA having certain mRNA purity levels are provided herein. In some embodiments, a composition comprises a lipid nanoparticle encapsulating a mRNA, wherein the composition comprises a mRNA purity level of greater than 50% main peak mRNA purity after at least thirty days of storage.

In some embodiments, the composition comprises a mRNA purity level of greater than 60% main peak mRNA purity after at least thirty days of storage. In some embodiments, the composition comprises a mRNA purity level of greater than 70% main peak mRNA purity after at least thirty days of storage. In some embodiments, the composition comprises a mRNA purity level of greater than 80% main peak mRNA purity after at least thirty days of storage. In some embodiments, the composition comprises a mRNA purity level of greater than 90% main peak mRNA purity after at least thirty days of storage. In some embodiments, the composition comprises a mRNA purity level of greater than 50% main peak mRNA purity after at least six months of storage. In some embodiments, the composition comprises a mRNA purity level of greater than 50% main peak mRNA purity after at least twelve months of storage.

In some embodiments, the storage is at room temperature. In some embodiments, the storage is at greater than room temperature. In some embodiments, the storage is at 4° C.

In some embodiments, the storage is in the range of 0-40° C., for example, 0-30° C., 0-25° C., 0-20° C., 0-15° C., 0-10° C., or 0-5° C. In certain embodiments, the storage is in the range of 2-10° C., 2-8° C., 4-8° C., or 4-6° C.

In certain embodiments, the composition comprises a mRNA purity level of greater than 50% main peak mRNA purity after at least twelve months of storage at 2-8° C.

In some embodiments, the composition comprises a compound of Formula I, or a tautomer or solvate thereof. In some embodiments, the composition comprises a compound of Formula II, or a tautomer or solvate thereof.

In some embodiments, the composition comprises a compound of Formula I, or a tautomer or solvate thereof, and comprises a mRNA purity level of greater than 50% main peak mRNA purity after at least twelve months of storage at 2-8° C. In some embodiments, the composition comprises a compound of Formula II, or a tautomer or solvate thereof, and comprises a mRNA purity level of greater than 50% main peak mRNA purity after at least twelve months of storage at 2-8° C.

According to some aspects, compositions of lipid nanoparticles encapsulating mRNA having certain compositions of RNA fragments are provided herein. In some embodiments, a composition comprises a lipid nanoparticle encapsulating a mRNA, wherein the composition comprises less than 50% RNA fragments after at least thirty days of storage. In some embodiments, the composition comprises less than 60% RNA fragments after at least thirty days of storage. In some embodiments, the composition comprises less than 70% RNA fragments after at least thirty days of storage. In some embodiments, the composition comprises less than 80% RNA fragments after at least thirty days of storage. In some embodiments, the composition comprises less than 90% RNA fragments after at least thirty days of storage. In some embodiments, the composition comprises less than 95% RNA fragments after at least thirty days of storage.

In some embodiments, the composition is stored for at least six months.

In some embodiments, the storage is at room temperature. In some embodiments, the storage is at greater than room temperature. In some embodiments, the storage is at 4° C.

In some embodiments, the composition comprises a compound of Formula I, or a tautomer or solvate thereof. In some embodiments, the composition comprises a compound of Formula II, or a tautomer or solvate thereof.

In some embodiments, the lipid nanoparticle comprises a ratio of 20-60% amino lipids, 5-30% phospholipid, 10-55% structural lipid, and 0.5-15% PEG-modified lipid. In some embodiments, the lipid nanoparticle comprises a ratio of 20-60% amino lipids, 5-25% phospholipid, 25-55% structural lipid, and 0.5-15% PEG-modified lipid. In certain embodiments, the ratio is a mass ratio. In other embodiments, the ratio is a molar ratio.

In certain embodiments, provided are compositions that do not contain a lipid component.

According to some aspects, methods for producing a protein in a subject are provided herein. In some embodiments, a method for producing a protein in a subject comprises administering a composition comprising a nucleic acid to a subject, wherein the nucleic acid is an mRNA and wherein the mRNA encodes for the production of a protein in the subject.

According to some aspects, devices enabling the use of compositions and methods disclosed herein are provided. In some embodiments, a syringe or cartridge, comprising a composition disclosed herein is provided. In some embodiments, an infusion pump, comprising a composition disclosed herein is provided. In some embodiments, a syringe or cartridge, comprising multiple doses of a composition disclosed herein is provided. In some embodiments, the syringe, cartridge, and/or infusion pump (including any associated components, such as tubing) are photoprotected (e.g., exclude or reduce sunlight, room light, UV light, and/or fluorescent light).

Other advantages and novel features will become apparent from the following detailed description of various non-limiting embodiments when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments are provided by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the subject matter. In the figures:

FIGS. 1A-1B show mRNA instability in lipid nanoparticle formulations at refrigerated temperature. In each case less than 9 months of refrigerated storage stability would be possible. FIG. 1A shows sized-based purity of formulation 1 over 12 months of refrigerated storage. FIG. 1B shows sized-based purity of formulation 2 over 6 months of refrigerated storage.

FIG. 2 shows mRNA purity as a function of time and berberine concentration.

FIG. 3 shows mRNA purity as a function of time and palmatine concentration.

FIG. 4 shows mRNA purity as a function of time and coralyne and palmatine concentration.

FIG. 5 shows HPLC analysis of the purity of mRNA stressed at 40° C. for 3 weeks in the absence of stabilizer (control) and in the presence of ˜1 mM berberine.

FIG. 6 shows dynamic light scattering analysis of the stability of mRNA LNP formulations incubated at 40° C. for 3 weeks in the absence of berberine and in the presence of berberine.

FIG. 7 shows mRNA purity as a function of temperature and pH in the absence of stabilizer, in the presence of 1 mM berberine, or in the presence of 1 mM palatine.

FIG. 8 shows mRNA purity as a function of berberine concentration and temperature after incubation at 40° C. for 1 week or 25° C. for 2 weeks.

FIG. 9 shows differential scanning calorimetry data for increasing amounts of palmatine chloride in combination with mRNA.

FIGS. 10A-B shows the stability of compositions comprising mRNA and palmatine chloride. FIG. 10A shows the stability of 0.20 mg/ml mRNA (empty circles), and 0.20 mg/ml mRNA+2 mM palmatine chloride (dark circles), at 5° C. over twelve months. The degradation rate (% per month) of the stabilized composition was 1.8% as compared to 5.4% for the unstabilized composition. The stabilized composition showed 20% greater purity after ten months. FIG. 10B shows the stability of 0.20 mg/ml mRNA (empty circles), and 0.20 mg/ml mRNA+2 mM palmatine chloride (dark circles), at 25° C. over six months. The degradation rate (% per month) of the stabilized composition was 8.6% as compared to 38% for the unstabilized composition. The stabilized composition showed 35% greater purity after six months.

FIG. 11 shows the internal concentration of palmatine chloride in lipid nanoparticles encapsulating mRNA over time at 5° C., 15° C., 25° C., or 40° C. Formulations contain 0.20 mg/ml mRNA DP with 2 mM palmatine chloride.

FIG. 12 shows the stability of lipid nanoparticles encapsulating mRNA with varying concentrations of palmatine chloride and DTPA.

### DETAILED DESCRIPTION

Lipid nanoparticle (LNP) formulations offer the opportunity to deliver various nucleic acids in vivo for applications in which unencapsulated nucleic acids would be ineffective, but their broad utility has been hindered by insufficient nucleic acid stability over relevant timeframes. Degradation of nucleic acids within LNP formulations limits the use of such formulations to applications in which frozen compositions are acceptable.

### Stabilizing Compounds

It was surprisingly found that a mixture of certain compounds with nucleic acids in LNP formulations or with LNP formulations resulted in substantially improved stability including nucleic acid stability. Accordingly, nucleic acid and lipid compositions, and methods for their preparation and use are provided.

It was determined, using both accelerated and real-time conditions, that the stability of formulations can be significantly enhanced using potent stabilizing excipients or compounds provided herein. The inclusion of these compounds in formulations such as lipid based and/or nucleic acid formulations provides properties useful for preparation, storage, and use of therapeutic agents. For instance, it has been demonstrated that for mRNA-lipid nanoparticle (mRNA-LNP) compositions, combination with stabilizing compounds (e.g., a compound of Formula I, of Formula II, or a tautomer or solvate thereof) dramatically inhibits the rate of purity loss of mRNA encapsulated within the LNP under a variety of storage conditions. The instability of mRNA, specifically loss of purity, is considered one of the greatest challenges to its fundamental therapeutic and commercial viability. Additionally, the instability of mRNA is significant when formulated as an LNP. Provided are stabilizing compounds that provide a solution to these problems.

The discovery that a class of compounds is able to stabilize nucleic acids within a lipid carrier such as an LNP is unexpected and unprecedented. This finding enables several significant applications, including extended refrigerated liquid shelf-life, extended in-use periods at room temperature, and extended in-use stability at physiological temperatures up to higher temperatures such as 40° C. Achieving a stable liquid formulation also enables commercially and therapeutically desirable packaging and delivery options including prefilled syringes and cartridges for patient-friendly autoinjector and infusion pump devices. The incorporation of this compound into methods of making as well as, optionally, the final drug product will provide a significant improvement in purity values of therapeutic nucleic acids, such as mRNA upon manufacture. This solves a critical problem, as current manufacturing processes and formulations experience a 5-10% purity loss during LNP formation and processing that is typical with current large-scale LNP production. The ability to stabilize solutions and pharmaceutical preparations of mRNA and other therapeutics therefore represent a valuable technology facilitating broader use of therapeutic compositions such as mRNA compositions.

Some aspects provide stabilized nucleic acid compositions comprising a nucleic acid and a compound of Formula I:

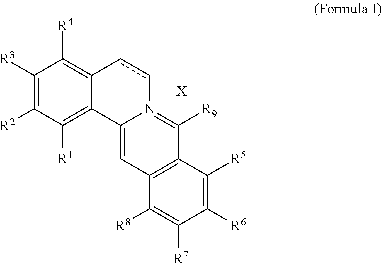

or a tautomer or solvate thereof. Some aspects provide stabilized nucleic acid compositions comprising a nucleic acid and a compound of Formula II:

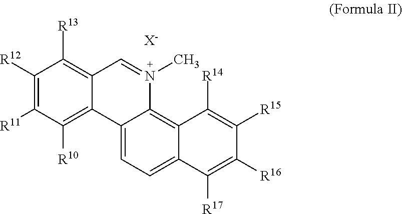

or a tautomer or solvate thereof.

The term “pharmaceutically acceptable anion” refers to a negatively charged group that is associated with a positively charged group (e.g., the polycyclic core of Formula I) in order to maintain electronic neutrality. Also referred to as a “counterion,” the anion may be monovalent (e.g., including one formal negative charge). The anion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, propanoate, benzoate, glycerate, lactate, tartrate, glycolate, gluconate, and the like). Exemplary counterions which may be multivalent include CO₃²⁻, HPO₄²⁻, PO₄³⁻, SO₄²⁻, and carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like). In certain particular embodiments, the pharmaceutically acceptable anion of a compound of Formulae I, Ia, Ib, Ic, II or IIa is chloride.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THE, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refer to isomers of a compound which differ only in the position of the protons and electrons, e.g., two or more interconvertible compounds resulting from at least one migration of a hydrogen atom or electron pair, and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may be catalyzed by acid or base. Exemplary tautomerizations include keto-to-enol, amide-to-imide, lactam-to-lactam, enamine-to-imine, and enamine-to-(a different enamine) tautomerizations. Tautomerizations may result from delocalization of electrons (e.g., between heteroatoms and/or pi bonds in conjugated systems).

In some embodiments, the compound (e.g., a compound of Formula I) has a purity of at least 50%. In some embodiments, the compound has a purity of at least 55%, at least 60%, at least 65%, at least 70%, at least 75%, at least 80%, at least 85%, at least 90%, at least 95%, at least 96%, at least 97%, at least 98%, at least 99%, or at least 99.9%. Exemplary methods of determining the purity of a compound are discussed below.

In some embodiments, the composition (e.g., a nucleic acid and/or lipid composition disclosed herein) has a purity of at least 50%. The purity of a composition reflects the amount of components used to make the composition in the composition at any particular point in time. In some embodiments, the composition has a purity of at least 55%, at least 60%, at least 65%, at least 70%, at least 75%, at least 80%, at least 85%, at least 90%, at least 95%, at least 96%, at least 97%, at least 98%, at least 99%, or at least 99.9%.

The purity of a composition may be characterized based on the presence of impurities in the composition at any particular point in time. Impurities include, for instance, lipid-RNA adducts, or elemental metals. In some embodiments, a composition is considered to have an adequate purity if less than 10% of the RNA in a composition is in the form of a lipid-RNA adduct. In some embodiments, a composition is considered to have an adequate purity if less than 9%, 8%, 7%, 6%, 5%, 4%, 3%, 2%, 1%, 0.5%, or 0.1% of the RNA in a composition is in the form of a lipid-RNA adduct.

The purity of a composition may also be characterized based on the presence of adduct impurities that arise from the decomposition of an ionizable amine lipid (e.g., tertiary amine lipid) component of the composition. For example, an ionizable amine lipid may be converted into one or more electrophilic compounds which react with nucleic acid bases to afford covalent adducts. In particular, an ionizable amine lipid may be oxidized to the corresponding N-oxide, which is ultimately hydrolyzed to the corresponding aldehyde. The aldehyde may then react with amine residues on the bases to form covalent adducts. In some embodiments, the use of a photoprotective container in the preparation or use of a composition comprising a stabilizing compound reduces the amount of reactive aldehyde.

In certain embodiments, oxidation of the ionizable amine is facilitated by mildly acidic conditions. In certain embodiments, oxidation of the ionizable amine is facilitated by a sensitizing compound. In some embodiments, the sensitizing compound is a triplet sensitizer. In some embodiments, the sensitizing compound is a compound of Formula I, of Formula I, or a tautomer or solvate thereof. In certain embodiments, oxidation of the ionizable amine is facilitated by exposure to light. In such embodiments, formation of covalent adducts can be reduced by reducing exposure of a composition (e.g., a lipid nanoparticle formulation) to light. In other embodiments, oxidation of the ionizable amine occurs without exposure to light.

In certain embodiments, the covalent adducts are distinguished as late-eluting peaks using reverse phase ion pair high performance liquid chromatography (RP-IP HPLC). In certain embodiments, the covalent adducts are not distinguishable using capillary electrophoresis.

The formation of covalent adducts to mRNA can abrogate the ability of the mRNA to undergo translation.

In some embodiments, the stabilizer compound (e.g., a compound of Formula I, of Formula II, or a tautomer or solvate thereof) preserves the purity of the nucleic acid. In some embodiments, the stabilizer compound preserves the purity of the nucleic acid in a concentration dependent manner. In some embodiments, 0.01-10 mM of stabilizer compound preserves the purity of the nucleic acid. In some embodiments, 0.1-1 mM of stabilizer compound preserves the purity of the nucleic acid. In some embodiments, 0.1-1 mM of stabilizer compound preserves the purity of the nucleic acid at 25° C. In some embodiments, the stabilizer compound decreases degradation of the nucleic acid. In some embodiments, the stabilizer compound decreases adduct formation.

The term “elemental metal” is given its ordinary meaning in the art. A metal is an element that readily forms positive ions (i.e., cations) and forms metallic bonds. An elemental metal refers to a metal which is not present in a salt form or otherwise within a compound. Those of ordinary skill in the art will, in general, recognize elemental metals. Elemental metals include Ca, Mg, Ti, Cr, Mn, Fe, V, Co, Cu, Ni, Zn, Mn, Fe, and/or Cd.

In some embodiments, the compound (e.g., a compound of Formula I, of Formula II, or a tautomer or solvate thereof) is free of elemental metals. In some embodiments, the compound contains fewer than 1000 ppm, fewer than 900 ppm, fewer than 800 ppm, fewer than 700 ppm, fewer than 600 ppm, fewer than 500 ppm, fewer than 400 ppm, fewer than 300 ppm, fewer than 200 ppm, fewer than 100 ppm, fewer than 90 ppm, fewer than 80 ppm, fewer than 70 ppm, fewer than 60 ppm, fewer than 50 ppm, fewer than 40 ppm, fewer than 30 ppm, fewer than 20 ppm, fewer than 10 ppm, fewer than 9 ppm, fewer than 8 ppm, fewer than 7 ppm, fewer than 6 ppm, fewer than 5 ppm, fewer than 4 ppm, fewer than 3 ppm, fewer than 2 ppm, fewer than 1 ppm of elemental metals. The proceeding values refer to the amount of any single elemental metal, or the total amount of more than one elemental metal.

In some embodiments, the compound (e.g., a compound of Formula I, of Formula II, or a tautomer or solvate thereof) contains fewer than 1000 ppm, fewer than 900 ppm, fewer than 800 ppm, fewer than 700 ppm, fewer than 600 ppm, fewer than 500 ppm, fewer than 400 ppm, fewer than 300 ppm, fewer than 200 ppm, fewer than 100 ppm, fewer than 90 ppm, fewer than 80 ppm, fewer than 70 ppm, fewer than 60 ppm, fewer than 50 ppm, fewer than 40 ppm, fewer than 30 ppm, fewer than 20 ppm, fewer than 10 ppm, fewer than 9 ppm, fewer than 8 ppm, fewer than 7 ppm, fewer than 6 ppm, fewer than 5 ppm, fewer than 4 ppm, fewer than 3 ppm, fewer than 2 ppm, fewer than 1 ppm of one or more metals selected from Ca, Mg, Ti, Cr, Mn, Fe, V, Co, Cu, Ni, Zn, Mn, Fe, and Cd.

In some embodiments, the compound contains fewer than 1000 ppm, fewer than 900 ppm, fewer than 800 ppm, fewer than 700 ppm, fewer than 600 ppm, fewer than 500 ppm, fewer than 400 ppm, fewer than 300 ppm, fewer than 200 ppm, fewer than 100 ppm, fewer than 90 ppm, fewer than 80 ppm, fewer than 70 ppm, fewer than 60 ppm, fewer than 50 ppm, fewer than 40 ppm, fewer than 30 ppm, fewer than 20 ppm, fewer than 10 ppm, fewer than 9 ppm, fewer than 8 ppm, fewer than 7 ppm, fewer than 6 ppm, fewer than 5 ppm, fewer than 4 ppm, fewer than 3 ppm, fewer than 2 ppm, fewer than 1 ppm of one or more metals selected from Fe, Cu, and Zn. In certain embodiments, the compound contains less than 10 ppm of Fe, Cu, and Zn. In other embodiments, the compound contains less than 1 ppm of Fe, Cu, and Zn.

Purity can be determined by any suitable method known in the art. Non-limiting examples of methods to determine the purity of a compound include melting point determination, boiling point determination, spectroscopy (e.g., UV-VIS spectroscopy), titration, chromatography (e.g., liquid chromatography or gas chromatography), mass spectroscopy, capillary electrophoresis, and optical rotation.

In some embodiments, the composition (e.g., a nucleic acid and/or lipid composition disclosed herein) comprises a chelator. A chelator may be a compound which forms two or more coordinate bonds to a metal atom. Chelators may comprise one or more chemical functional groups, such as amines and carboxylic acids, that promote such bonds. A chelator may form a stable, soluble complex with a metal atom. Complexation with a chelator may neutralize or attenuate the reactivity of a metal atom. The metal atom may be an elemental metal, as described herein. In certain embodiments, the chelator is a pharmaceutically acceptable chelator. Exemplary, non-limiting, examples of such chelators are EDTA (ethylenediaminetetraacetic acid) and DTPA (diethylenetriaminepentaacetic acid). In certain embodiments, the composition comprises 1 μM-100 mM chelator. For example, the composition may comprise 1-100 μM, 100-200 μM, 200-300 μM, 300-400 μM, 400-500 μM, 500-600 μM, 600-700 μM, 700-800 μM, 800-900 μM, or 900-950 μM, or 950 μM-1 mM chelator. The composition may comprise 1-100 mM chelator. For example, the composition may comprise 1-2 mM, 1-3 mM, 1-4 mM, 1-5 mM, 1-6 mM, 1-7 mM, 1-8 mM, 1-9 mM, or 1-10 mM chelator. The composition may comprise 10-20 mM, 20-30 mM, 30-40 mM, 40-50 mM, 50-60 mM, 60-70 mM, 70-80 mM, 80-90 mM, or 90-100 mM chelator. In certain embodiments, the composition comprises about 1 mM chelator. In certain embodiments, the stability of a composition is increased or improved with the addition of a chelator.

According to some embodiments, compositions are formulated in aqueous solutions. An aqueous solution is a solution in which components are dissolved or otherwise dispersed within water.

In some embodiments, an aqueous solution has a given pH value. In some embodiments, the pH of an aqueous solution is within the range of about 4.5 to about 8.5. In some embodiments, the pH of an aqueous solution is within the range of about 5 to about 8, about 6 to about 8, about 7 to about 8, about 6.5 to about 8, about 6.5 to about 7.5, about 6.5 to about 7, about 7.5 to about 8.5, or any range or combination thereof. In some embodiments, the pH of an aqueous solution is or is about 5, is or is about 5.5, is or is about 6, is or is about 6.5, is or is about 7, is or is about 7.5, or is or is about 8. In some embodiments, the pH of an aqueous solution is about 6.

In some embodiments, an aqueous solution comprises a pH buffer component, such as a phosphate buffer, a tris buffer, an acetate buffer, a histidine buffer or a citrate buffer, among others. Such a buffer acts to modulate the pH of an aqueous solution, such as an aqueous solution having a pH of 5, 5.5, 6, 6.5, 7, 7.5 or 8. In some embodiments, the aqueous solution has a buffered pH of about 6. Aqueous solutions may comprise various concentrations of salts (e.g., sodium chloride, NaCl). In some embodiments, an aqueous solution may comprise a salt (e.g., NaCl) in a concentration of or about 50 mM, of or about 60 mM, of or about 70 mM, of or about 80 mM, of or about 90 mM, of or about 100 mM, of or about 110 mM, of or about 120 mM, of or about 130 mM, of or about 140 mM, of or about 150 mM, of or about 160 mM, of or about 170 mM, of or about 180 mM, of or about 190 mM, of or about 200 mM, or any intermediate concentration therein. In embodiments in which an aqueous solution comprises more than one salt, each salt may independently have a concentration of one or more of the values described above.

According to some aspects, aqueous solutions (e.g., aqueous solutions comprising nucleic acid, lipid, or nucleic acid and lipid) comprise a compound (e.g., a compound of Formula I, of Formula II, or a tautomer or solvate thereof) at a concentration of between about 0.1 mM and about 10 mM. In some embodiments, an aqueous solution (e.g., an aqueous solution comprising nucleic acid, lipid, or nucleic acid and lipid) comprises a compound (e.g., a compound of Formula I, of Formula II, or a tautomer or solvate thereof) at a concentration of between about 0.2 mM and about 10 mM, about 0.3 mM and about 10 mM, about 0.4 mM and about 10 mM, about 0.5 mM and about 10 mM, about 0.6 mM and about 10 mM, about 0.7 mM and about 10 mM, about 0.8 mM and about 10 mM, about 0.9 mM and about 10 mM, about 1 mM and about 10 mM, about 0.5 mM and about 9 mM, about 0.5 mM and about 8 mM, about 0.5 mM and about 7 mM, about 0.5 mM and about 6 mM, about 0.5 mM and about 5 mM, about 0.5 mM and about 4 mM, about 0.5 mM and about 3 mM, about 0.5 mM and about 2 mM, about 0.5 mM and about 1.5 mM, about 0.5 mM and about 1 mM, or any range or combination thereof. In some embodiments, an aqueous solution (e.g., an aqueous solution comprising nucleic acid, lipid, or nucleic acid and lipid) comprises a compound (e.g., a compound of Formula I, of Formula II, or a tautomer or solvate thereof) at a concentration of or about 0.1 mM, 0.2 mM, 0.3 mM, 0.4 mM, 0.5 mM, 0.6 mM, 0.7 mM, 0.8 mM, 0.9 mM, 1 mM, 1.5 mM, 2 mM, 2.5 mM, 3 mM, 4 mM, 5 mM, 6 mM, 7 mM, 8 mM, 9 mM, or of or about 10 mM. In some embodiments, an aqueous solution (e.g., an aqueous solution comprising nucleic acid, lipid, or nucleic acid and lipid) comprises a compound (e.g., a compound of Formula I, of Formula II, or a tautomer or solvate thereof) at a concentration of or about 0.5 mM, 1 mM, 1.5 mM, or of or about 2 mM. In some embodiments, an aqueous solution (e.g., an aqueous solution comprising nucleic acid, lipid, or nucleic acid and lipid) does not comprise a compound of Formula I, of Formula II, or a tautomer or solvate thereof.

According to some aspects, a composition is a lyophilized product. A lyophilized product is one from which liquid (e.g., water) has been removed by freeze drying, in which a liquid product is frozen and subsequently placed under a vacuum to remove liquid, leaving a composition substantially free of liquid. In some embodiments, a lyophilized product comprises lipids. In some embodiments, a lyophilized product comprises lipid nanoparticles. In some embodiments, a lyophilized product comprises nucleic acid. In some embodiments, a lyophilized product comprises nucleic acid encapsulated within lipid nanoparticles. In some embodiments, a lyophilized product comprises a compound of Formula I, of Formula II, or a tautomer or solvate thereof. In some embodiments, a lyophilized product comprises a compound of Formula I, of Formula II, or a tautomer or solvate thereof. In some embodiments, a lyophilized product comprises lipids, nucleic acids, a compound of Formula I, of Formula II, or a tautomer or solvate thereof, or any mixture thereof. In some embodiments, a lyophilized product is reconstituted with a solution comprising a compound of Formula I, of Formula II, or a tautomer or solvate thereof.

According to some aspects, a compound (e.g., a compound of Formula I, of Formula II, or a tautomer or solvate thereof) permeates into a lipid nanostructure (e.g., lipid nanoparticle, liposome, or lipoplex) to some extent. Permeation into a lipid nanostructure can be characterized, for example, by a partition coefficient representing the relative concentrations at equilibrium of the compound in the lipid nanostructure and in the solution in which the lipid nanostructure is comprised. The partition coefficient is a ratio of concentrations, and therefore represents the relative solubilities of the compound in the bulk solution and in the lipid nanostructure. A partition coefficient can be determined by one of skill in the art, for example by equilibrium dialysis.

In some embodiments, permeation of the compound into a lipid nanostructure (e.g., lipid nanoparticle, liposome, or lipoplex) is defined by a partition coefficient K_{LS} representing the partitioning between a solution (e.g., water or an aqueous solution) and the lipid nanostructure comprised within the solution. In some embodiments, the log of the partition coefficient K_{LS} (log K_{LS}) of a compound provided herein for a solution provided herein and a lipid nanostructure provided herein, measured at 25° C. is or is about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10. In some embodiments, the log K_{LS} is defined with reference to a compound (e.g., a compound of Formula I, of Formula II, or a tautomer or solvate thereof) in water partitioning into a lipid nanostructure. In some embodiments, permeation of the compound into a lipid nanostructure (e.g., lipid nanoparticle, liposome, or lipoplex) is defined by a partition coefficient K_{OW} which is defined by the ratio of concentrations of the compound in octanol and water at equilibrium. In some embodiments, the log of the partition coefficient K_{OW} (log K_{OW}) of a compound disclosed herein, measured at 25° C. is or is about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10. In some embodiments, log K_{OW} of a compound disclosed herein, measured at 25° C., is or is about 6. In some embodiments, log K_{OW} of a compound disclosed herein, measured at 25° C., is or is about 5.85. In some embodiments, log K_{OW} of a compound disclosed herein, measured at 25° C., is or is about 5.

In some embodiments, permeation of a compound (e.g., a compound of Formula I, of Formula II, or a tautomer or solvate thereof) into a lipid nanostructure (e.g., lipid nanoparticle, liposome, or lipoplex) is defined by the amount of the compound (e.g., by weight) present in the lipid nanostructure following incubation of the lipid nanostructure with a given concentration of the compound. In some embodiments, permeation of a compound into a lipid nanostructure is also defined by the temperature at which the incubation of the lipid nanostructure with the compound is conducted. In some embodiments, following incubation of a lipid nanostructure disclosed herein with a 1 mM solution of the compound disclosed herein, the lipid nanostructure comprises 0.001%, 0.005%, 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 1.1%, 1.12%, 1.14%, 1.16%, 1.18%, 1.2%, 1.22%, 1.24%, 1.26%, 1.28%, 1.3%, 1.32%, 1.34%, 1.36%, 1.38%, 1.4%, 1.42%, 1.44%, 1.46%, 1.48%, 1.5%, 1.52%, 1.54%, 1.56%, 1.58%, 1.6%, 1.62%, 1.64%, 1.66%, 1.68%, 1.7%, 1.72%, 1.74%, 1.76%, 1.78%, 1.8%, 1.82%, 1.84%, 1.86%, 1.88%, 1.9%, 1.92%, 1.94%, 1.96%, 1.98%, 2%, 2.2%, 2.4%, 2.6%, 2.8%, 3% by weight of the compound, or any range or combination thereof.

In some embodiments, following incubation of a lipid nanostructure disclosed herein with a 1 mM solution of the compound disclosed herein, the permeated concentration of the compound is 0.01-1 mM. In certain embodiments, the permeated concentration of the compound is about 0.01-0.05 mM, 0.05-0.1 mM, 0.1-0.15 mM, 0.15-0.2 mM, 0.2-0.25 mM, 0.25-0.3 mM, 0.3-0.35 mM, 0.35-0.4 mM, 0.4-0.45 mM, 0.45-0.5 mM, 0.5-0.55 mM, 0.55-0.6 mM, 0.6-0.65 mM, 0.65-0.7 mM, 0.7-0.75 mM, 0.75-0.8 mM, 0.8-0.85 mM, 0.85-0.9 mM, 0.9-0.95 mM, or 0.95-1 mM.

In some embodiments, following incubation of a lipid nanostructure disclosed herein with a 2 mM solution of the compound disclosed herein, the lipid nanostructure comprises 0.001%, 0.005%, 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 1.1%, 1.12%, 1.14%, 1.16%, 1.18%, 1.2%, 1.22%, 1.24%, 1.26%, 1.28%, 1.3%, 1.32%, 1.34%, 1.36%, 1.38%, 1.4%, 1.42%, 1.44%, 1.46%, 1.48%, 1.5%, 1.52%, 1.54%, 1.56%, 1.58%, 1.6%, 1.62%, 1.64%, 1.66%, 1.68%, 1.7%, 1.72%, 1.74%, 1.76%, 1.78%, 1.8%, 1.82%, 1.84%, 1.86%, 1.88%, 1.9%, 1.92%, 1.94%, 1.96%, 1.98%, 2%, 2.2%, 2.4%, 2.6%, 2.8%, 3% by weight of the compound, or any range or combination thereof.

In some embodiments, following incubation of a lipid nanostructure disclosed herein with a 2 mM solution of the compound disclosed herein, the permeated concentration of the compound is 0.01-2 mM. In certain embodiments, the permeated concentration of the compound is about 0.01-0.05 mM, 0.05-0.1 mM, 0.1-0.15 mM, 0.15-0.2 mM, 0.2-0.25 mM, 0.25-0.3 mM, 0.3-0.35 mM, 0.35-0.4 mM, 0.4-0.45 mM, 0.45-0.5 mM, 0.5-0.55 mM, 0.55-0.6 mM, 0.6-0.65 mM, 0.65-0.7 mM, 0.7-0.75 mM, 0.75-0.8 mM, 0.8-0.85 mM, 0.85-0.9 mM, 0.9-0.95 mM, 0.95-1 mM, 1-1.05 mM, 1.05-1.1 mM, 1.1-1.15 mM, 1.15-1.2 mM, 1.2-1.25 mM, 1.25-1.3 mM, 1.3-1.35 mM, 1.35-1.4 mM, 1.4-1.45 mM, 1.45-1.5 mM, 1.5-1.55 mM, 1.55-1.6 mM, 1.6-1.65 mM, 1.65-1.7 mM, 1.7-1.75 mM, 1.75-1.8 mM, 1.8-1.85 mM, 1.85-1.9 mM, 1.9-1.95 mM, or 1.95-2 mM.

In some embodiments, permeability increases at the gel-to-liquid phase transition. In some embodiments, permeability increases with greater surface fluidity (e.g., membrane viscosity). In some embodiments, surface fluidity increases with temperature. In some embodiments, the LNPs do not undergo a phase transition. In some embodiments, surface polarity (e.g., the presence of polar molecules) increases with temperature.

In some embodiments, the surface properties of the LNP change over time in the absence of stabilizer (e.g., a compound of Formula I, of Formula II, or a tautomer or solvate thereof). In some embodiments, the surface polarity of the LNP increases over time in the absence of stabilizer. In some embodiments, the surface fluidity of the LNP increases over time in the absence of stabilizer. In some embodiments, the surface fluidity of the LNP stays approximately constant over time in the absence of stabilizer.

In some embodiments, the surface properties of the LNP change over time in the presence of stabilizer (e.g., a compound of Formula I, of Formula II, or a tautomer or solvate thereof). In some embodiments, the surface polarity of the LNP increases over time in the presence of stabilizer. In some embodiments, the surface polarity of the LNP increases more over time in the presence of stabilizer than in the absence of stabilizer. In some embodiments, the surface fluidity of the LNP increases over time in the presence of stabilizer. In some embodiments, the surface fluidity of the LNP increases less over time in the presence of stabilizer than in the absence of stabilizer.

In some embodiments, the nanoparticle and nucleic acid are equilibrated at 5° C. to 60° C. In some embodiments, the nanoparticle and nucleic acid are equilibrated at 5, 15, 25, 32, 40, 50, or 60° C. In some embodiments, the nanoparticle and nucleic acid are equilibrated with a stabilizer. In some embodiments, the nanoparticle and nucleic acid are equilibrated for at least 10 minutes.

In some embodiments, a stabilizing compound (e.g., a compound of Formula I, of Formula II, or a tautomer or solvate thereof) is incubated with the mRNA (e.g., a LNP comprising the mRNA) for a period of at least 30 minutes, such as 1 hour, 2 hours, 3 hours, 4 hours, 5 hours, 6 hours, 7 hours, 8 hours, 9 hours, 10 hours, 15 hours, 20 hours, 24 hours, 36 hours, 48 hours, 72 hours, 4 days, 5 days, 6 days, 7 days, 8 days, 9 days, 10 days or more. In some embodiments, the stabilizing compound is incubated with the mRNA (e.g., a LNP comprising the mRNA) at a temperature of 0° C. or more, such as 5° C., 10° C., 15° C., 20° C., 25° C., 30° C., 35° C., 37° C., 40° C., 45° C., or more.

In some embodiments, a compound disclosed herein is water soluble. In some embodiments, the compound has a solubility in water of at least 10 mg/L (e.g., at least 100 mg/L, at least 200 mg/L, at least 300 mg/L, at least 400 mg/L, at least 500 mg/L, at least 600 mg/L, at least 700 mg/L, at least 800 mg/L, at least 900 mg/L, at least 1 g/L, at least 2 g/L, at least 3 g/L, at least 10 g/L, or more) at 25° C. In some embodiments, the compound has a solubility in water of or about 50 g/L at 25° C. In some embodiments, the compound has a solubility in water of or about 45 g/L at 25° C. In some embodiments, the compound has a solubility in water of or about 43.6 g/L at 25° C.

According to some aspects, a compound (e.g., a compound of Formula I, of Formula II, or a tautomer or solvate thereof) disclosed herein has a low cytotoxicity. In some embodiments, a compound disclosed herein has a cytotoxicity LC50 value of at least 5 mg/L (e.g., at least 10 mg/L, at least 15 mg/L, at least 20 mg/L, at least 25 mg/L, at least 30 mg/L, at least 35 mg/L, at least 40 mg/L, at least 45 mg/L, at least 50 mg/L, or more) when measured in mammalian cells (e.g., human cells or murine cells) in culture, or when measured in test organisms (e.g., fish, such as zebrafish or Mystus vittatus).

In some embodiments, the stabilizing compound is a photosensitive stabilizing compound. Thus, in some embodiments the stabilizing compound is shielded from light exposure (e.g., sunlight, room light, UV light, and/or fluorescent light). Thus, in some embodiments, the stabilizing compound and/or mixtures or composition containing the stabilizing compound (e.g., mRNA compositions, LNP compositions, mRNA-encapsulated LNP compositions, and/or LNP component compositions) are protected from exposure to light. That is, in some embodiments light is inhibited or prohibited from contacting a composition comprising the stabilizing compound. In some embodiments, the composition comprising the stabilizer compound is stored in a container that inhibits or prohibits light from contacting the composition. In some embodiments, the composition is stored in a container covered mostly, or preferably entirely, with a film, foil, or coating that is light impermeable.

### Nucleic Acids

Also provided are stabilized nucleic acids. In some embodiments, the nucleic acids are in contact with a stabilizing compound. Some embodiments comprise a composition comprising a nucleic acid and a stabilizing compound.

The term “nucleic acid” refers to multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G))). As used herein, the term nucleic acid refers to polyribonucleotides as well as polydeoxyribonucleotides. The term nucleic acid also includes polynucleosides (i.e., a polynucleotide minus the phosphate) and any other organic base containing polymer. Non-limiting examples of nucleic acids include chromosomes, genomic loci, genes or gene segments that encode polynucleotides or polypeptides, coding sequences, non-coding sequences (e.g., intron, 5′-UTR, or 3′-UTR) of a gene, pri-mRNA, pre-mRNA, cDNA, mRNA, etc. In some embodiments, the nucleic acid is mRNA. A nucleic acid may include a substitution and/or modification. In some embodiments, the substitution and/or modification is in one or more bases and/or sugars. For example, in some embodiments a nucleic acid includes nucleic acids having backbone sugars that are covalently attached to low molecular weight organic groups other than a hydroxyl group at the 2′ position and other than a phosphate group or hydroxy group at the 5′ position. Thus, in some embodiments, a substituted or modified nucleic acid includes a 2′-O-alkylated ribose group. In some embodiments, a modified nucleic acid includes sugars such as hexose, 2′-F hexose, 2′-amino ribose, constrained ethyl (cEt), locked nucleic acid (LNA), arabinose or 2′-fluoroarabinose instead of ribose. Thus, in some embodiments, a nucleic acid is heterogeneous in backbone composition thereby containing any possible combination of polymer units linked together such as peptide-nucleic acids (which have an amino acid backbone with nucleic acid bases).

In some embodiments, a nucleic acid is DNA, RNA, PNA, cEt, LNA, ENA or hybrids including any chemical or natural modification thereof. Chemical and natural modifications are well known in the art. Non-limiting examples of modifications include modifications designed to increase translation of the nucleic acid, to increase cell penetration or sub-cellular distribution of the nucleic acid, to stabilize the nucleic acid against nucleases and other enzymes that degrade or interfere with the structure or activity of the nucleic acid, and to improve the pharmacokinetic properties of the nucleic acid.

In some embodiments, the compositions comprise a RNA having an open reading frame (ORF) encoding a polypeptide. In some embodiments, the RNA is a messenger RNA (mRNA). In some embodiments, the RNA (e.g., mRNA) further comprises a 5′ UTR, 3′ UTR, a poly(A) tail and/or a 5′ cap analog.

Messenger RNA (mRNA) is any RNA that encodes a (at least one) protein (e.g., a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents RNA (e.g., mRNA), the “T”'s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in an RNA polynucleotide.

Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a composition includes an RNA polynucleotide having an open reading frame encoding at least one polypeptide having at least one modification, at least one 5′ terminal cap, and is formulated within a lipid nanoparticle along with the stabilizing compound (e.g., a compound of Formula I, of Formula II, or a tautomer or solvate thereof).

5′ terminal caps can include endogenous caps or cap analogs. According to the present invention, a 5′ terminal cap can comprise a guanine analog. Useful guanine analogs include, but are not limited to, inosine, NI-methyl-guanosine, 2′fluoro-guanosine, 7-deaza-guanosine, 8-oxo-guanosine, 2-amino-guanosine, LNA-guanosine, and 2-azido-guanosine.

Also provided herein are exemplary caps including those that can be used in co-transcriptional capping methods for ribonucleic acid (RNA) synthesis, using RNA polymerase, e.g., wild type RNA polymerase or variants thereof, e.g., such as those variants described herein. In one embodiment, caps can be added when RNA is produced in a “one-pot” reaction, without the need for a separate capping reaction. Thus, the methods, in some embodiments, comprise reacting a polynucleotide template with a RNA polymerase variant, nucleoside triphosphates, and a cap analog under in vitro transcription reaction conditions to produce RNA transcript.

In some embodiments, the cap analog binds to a polynucleotide template that comprises a promoter region comprising a transcriptional start site having a first nucleotide at nucleotide position +1, a second nucleotide at nucleotide position +2, and a third nucleotide at nucleotide position +3. In some embodiments, the cap analog hybridizes to the polynucleotide template at least at nucleotide position +1, such as at the +1 and +2 positions, or at the +1, +2, and +3 positions.

A cap analog may be, for example, a dinucleotide cap, a trinucleotide cap, or a tetranucleotide cap. In some embodiments, a cap analog is a dinucleotide cap. In some embodiments, a cap analog is a trinucleotide cap. In some embodiments, a cap analog is a tetranucleotide cap. As used here the term “cap” includes the inverted G nucleotide and can comprise additional nucleotides 3′ of the inverted G, e.g., 1, 2, or more nucleotides 3′ of the inverted G and 5′ to the 5′ UTR.

Exemplary caps comprise a sequence GG, GA, or GGA wherein the underlined, italicized G is an inverted G.

A trinucleotide cap, in some embodiments, comprises a compound of formula (I)

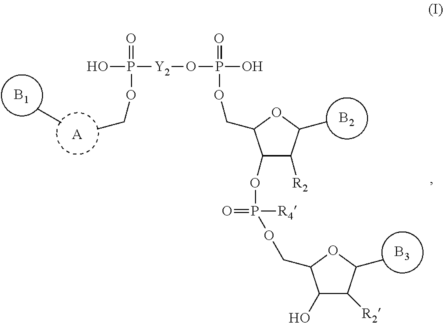

or a stereoisomer, tautomer or salt thereof, wherein

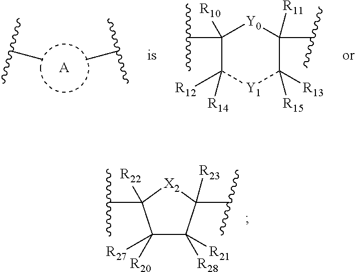

ring B₁ is a modified or unmodified Guanine;
ring B₂ and ring B₃ each independently is a nucleobase or a modified nucleobase;
X₂ is O, S(O)ₚ, NR₂₄ or CR₂₅R₂₆ in which p is 0, 1, or 2;
Y₀ is O or CR₆R₇;
Y1 is O, S(O)ₙ, CR₆R₇, or NR₈, in which n is 0, 1, or 2;
each [symbol] is a single bond or absent, wherein when each [symbol] is a single bond, Yi is O, S(O)ₙ, CR₆R₇, or NR₈; and when each [symbol] is absent, Y₁ is void;
Y₂ is (OP(O)R₄)ₘ in which m is 0, 1, or 2, or —O—(CR₄₀R₄₁)u-Q₀-(CR₄₂R₄₃)v-, in which Q₀ is a bond, O, S(O)ᵣ, NR₄₄, or CR₄₅R₄₆, r is 0, 1, or 2, and each of u and v independently is 1, 2, 3 or 4;
each R₂ and R_{2′} independently is halo, LNA, or OR₃;
each R₃ independently is H, C₁-C₆ alkyl, C₂-C₆ alkenyl, or C₂-C₆ alkynyl and R₃, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;
each R₄ and R₄′ independently is H, halo, C₁-C₆ alkyl, OH, SH, SeH, or BH₃⁻;
each of R₆, R₇, and R₈, independently, is -Q₁-T₁, in which Q₁ is a bond or C₁-C₃ alkyl linker optionally substituted with one or more of halo, cyano, OH and C₁-C₆ alkoxy, and T₁ is H, halo, OH, COOH, cyano, or Rₛ₁, in which Rₛ₁ is C₁-C₃ alkyl, C₂-C₆ alkenyl, C₂-C₆ alkynyl, C₁-C₆ alkoxyl, C(O)O—C₁-C₆ alkyl, C₃-C₈ cycloalkyl, C₆-C₁₀ aryl, NR₃₁R₃₂, (NR₃₁R₃₂R₃₃)⁺, 4 to 12-membered heterocycloalkyl, or 5- or 6-membered heteroaryl, and Rₛ₁ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered heterocycloalkyl, and 5- or 6-membered heteroaryl;
each of R₁₀, R₁₁, R₁₂, R₁₃ R₁₄, and R₁₅, independently, is -Q₂-T₂, in which Q₂ is a bond or C₁-C₃ alkyl linker optionally substituted with one or more of halo, cyano, OH and C₁-C₆ alkoxy, and T₂ is H, halo, OH, NH₂, cyano, NO₂, N₃, Rₛ₂, or ORₛ₂, in which Rₛ₂ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, NR₃₁R₃₂, (NR₃₁R₃₂R₃₃)⁺, 4 to 12-membered heterocycloalkyl, or 5- or 6-membered heteroaryl, and Rₛ₂ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered heterocycloalkyl, and 5- or 6-membered heteroaryl; or alternatively R₁₂ together with R₁₄ is oxo, or R₁₃ together with R₁₅ is oxo,
each of R₂₀, R₂₁, R₂₂, and R₂₃ independently is -Q₃-T₃, in which Q₃ is a bond or C₁-C₃ alkyl linker optionally substituted with one or more of halo, cyano, OH and C₁-C₆ alkoxy, and T₃ is H, halo, OH, NH₂, cyano, NO₂, N₃, R_{S3}, or OR_{S3}, in which R_{S3} is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, mono-C₁-C₆ alkylamino, di-C₁-C₆ alkylamino, 4 to 12-membered heterocycloalkyl, or 5- or 6-membered heteroaryl, and R_{S3} is optionally substituted with one or more substituents selected from the group consisting of halo, OH, oxo, C₁-C₆ alkyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl;
   each of R₂₄, R₂₅, and R₂₆ independently is H or C₁-C₆ alkyl;
each of R₂₇ and R₂₈ independently is H or OR₂₉; or R₂₇ and R₂₈ together form O—R₃₀—O; each R₂₉ independently is H, C₁-C₆ alkyl, C₂-C₆ alkenyl, or C₂-C₆ alkynyl and R₂₉, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;
R₃₀ is C₁-C₆ alkylene optionally substituted with one or more of halo, OH and C₁-C₆ alkoxyl;
each of R₃₁, R₃₂, and R₃₃, independently is H, C₁-C₆ alkyl, C₃-C₈ cycloalkyl, C₆-C₁₀ aryl, 4 to 12-membered heterocycloalkyl, or 5- or 6-membered heteroaryl;
each of R₄₀, R₄₁, R₄₂, and R₄₃ independently is H, halo, OH, cyano, N₃, OP(O)R₄₇R₄₈, or C₁-C₆ alkyl optionally substituted with one or more OP(O)R₄₇R₄₈, or one R₄₁ and one R₄₃, together with the carbon atoms to which they are attached and Q₀, form C₄-C₁₀ cycloalkyl, 4- to 14-membered heterocycloalkyl, C₆-C₁₀ aryl, or 5- to 14-membered heteroaryl, and each of the cycloalkyl, heterocycloalkyl, phenyl, or 5- to 6-membered heteroaryl is optionally substituted with one or more of OH, halo, cyano, N₃, oxo, OP(O)R₄₇R₄₈, C₁-C₆ alkyl, C₁-C₆ haloalkyl, COOH, C(O)O—C₁-C₆ alkyl, C₁-C₆ alkoxyl, C₁-C₆ haloalkoxyl, amino, mono-C₁-C₆ alkylamino, and di-C₁-C₆ alkylamino;

R⁴⁴ is H, C₁-C₆ alkyl, or an amine protecting group;

each of R₄₅ and R₄₆ independently is H, OP(O)R₄₇R₄₈, or C₁-C₆ alkyl optionally substituted with one or more OP(O)R₄₇R₄₈, and
each of R₄₇ and R₄₈, independently is H, halo, C₁-C₆ alkyl, OH, SH, SeH, or BH₃⁻.

It should be understood that a cap analog, as provided herein, may include any of the cap analogs described in international publication WO 2017/066797, published on 20 Apr. 2017, incorporated by reference herein in its entirety.

In some embodiments, the B₂ middle position can be a non-ribose molecule, such as arabinose.

In some embodiments R₂ is ethyl-based.

Thus, in some embodiments, a trinucleotide cap comprises the following structure:

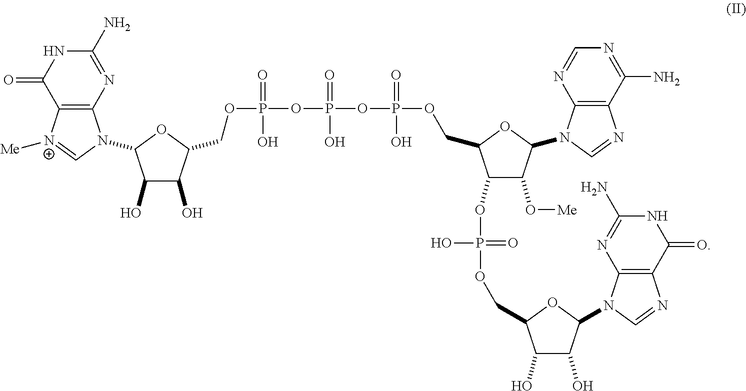

In yet other embodiments, a trinucleotide cap comprises the following structure:

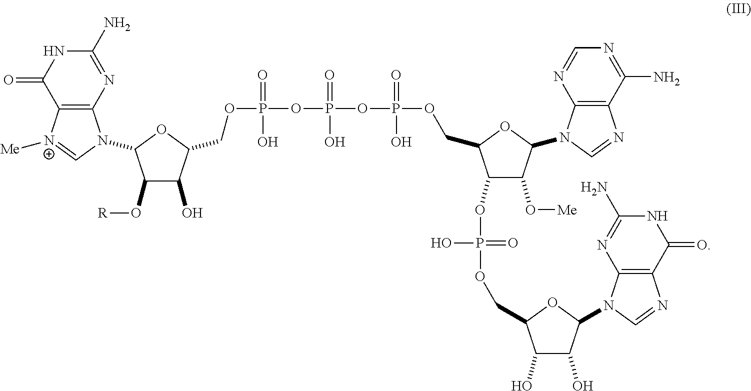

In still other embodiments, a trinucleotide cap comprises the following structure:

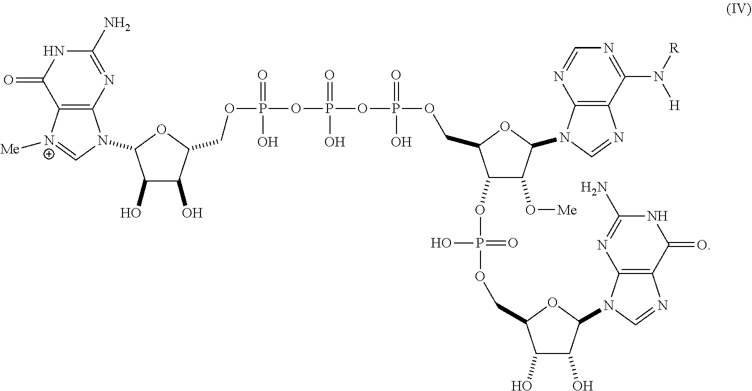

In some embodiments, R is an alkyl (e.g., C₁-C₆ alkyl). In some embodiments, R is a methyl group (e.g., C₁ alkyl). In some embodiments, R is an ethyl group (e.g., C₂ alkyl).

A trinucleotide cap, in some embodiments, comprises a sequence selected from the following sequences: GAA, GAC, GAG, GAU, GCA, GCC, GCG, GCU, GGA, GGC, GGG, GGU, GUA, GUC, GUG, and GUU. In some embodiments, a trinucleotide cap comprises GAA. In some embodiments, a trinucleotide cap comprises GAC. In some embodiments, a trinucleotide cap comprises GAG. In some embodiments, a trinucleotide cap comprises GAU. In some embodiments, a trinucleotide cap comprises GCA. In some embodiments, a trinucleotide cap comprises GCC. In some embodiments, a trinucleotide cap comprises GCG. In some embodiments, a trinucleotide cap comprises GCU. In some embodiments, a trinucleotide cap comprises GGA. In some embodiments, a trinucleotide cap comprises GGC. In some embodiments, a trinucleotide cap comprises GGG. In some embodiments, a trinucleotide cap comprises GGU. In some embodiments, a trinucleotide cap comprises GUA. In some embodiments, a trinucleotide cap comprises GUC. In some embodiments, a trinucleotide cap comprises GUG. In some embodiments, a trinucleotide cap comprises GUU.

In some embodiments, a trinucleotide cap comprises a sequence selected from the following sequences: m⁷GpppApA, m⁷GpppApC, m⁷GpppApG, m⁷GpppApU, m⁷GpppCpA, m⁷GpppCpC, m⁷GpppCpG, m⁷GpppCpU, m⁷GpppGpA, m⁷GpppGpC, m⁷GpppGpG, m⁷GpppGpU, m⁷GpppUpA, m⁷GpppUpC, m⁷GpppUpG, and m⁷GpppUpU.

In some embodiments, a trinucleotide cap comprises m⁷GpppApA. In some embodiments, a trinucleotide cap comprises m⁷GpppApC. In some embodiments, a trinucleotide cap comprises m⁷GpppApG. In some embodiments, a trinucleotide cap comprises m⁷GpppApU. In some embodiments, a trinucleotide cap comprises m⁷GpppCpA. In some embodiments, a trinucleotide cap comprises m⁷GpppCpC. In some embodiments, a trinucleotide cap comprises m⁷GpppCpG. In some embodiments, a trinucleotide cap comprises m⁷GpppCpU. In some embodiments, a trinucleotide cap comprises m⁷GpppGpA. In some embodiments, a trinucleotide cap comprises m⁷GpppGpC. In some embodiments, a trinucleotide cap comprises m⁷GpppGpG. In some embodiments, a trinucleotide cap comprises m⁷GpppGpU. In some embodiments, a trinucleotide cap comprises m⁷GpppUpA. In some embodiments, a trinucleotide cap comprises m⁷GpppUpC. In some embodiments, a trinucleotide cap comprises m⁷GpppUpG. In some embodiments, a trinucleotide cap comprises m⁷GpppUpU.

A trinucleotide cap, in some embodiments, comprises a sequence selected from the following sequences: m⁷G_{3′OMe}pppApA, m⁷G_{2′OMe}pppApC, m⁷G_{3′OMe}pppApG, m⁷G_{3′OMe}pppApU, m⁷G_{3′OMe}pppCpA, m⁷G_{3′OMe}pppCpC, m⁷G_{3′OMe}pppCpG, m⁷G_{3′OMe}pppCpU, m⁷G_{3′OMe}pppGpA, m⁷G_{3′OMe}pppGpC, m⁷G_{3′OMe}pppGpG, m⁷G_{3′OMe}pppGpU, m⁷G_{3′OMe}pppUpA, m⁷G_{3′OMe}pppUpC, m⁷G_{3′OMe}pppUpG, and m⁷G_{3′OMe}pppUpU.

In some embodiments, a trinucleotide cap comprises m⁷G_{3′OMe}pppApA. In some embodiments, a trinucleotide cap comprises m⁷G_{3′OMe}pppApC. In some embodiments, a trinucleotide cap comprises m⁷G_{2′OMe}pppApG. In some embodiments, a trinucleotide cap comprises m⁷G_{3′OMe}pppApU. In some embodiments, a trinucleotide cap comprises m⁷G_{3′OMe}pppCpA. In some embodiments, a trinucleotide cap comprises m⁷G_{3′OMe}pppCpC. In some embodiments, a trinucleotide cap comprises m⁷G_{2′OMe}pppCpG. In some embodiments, a trinucleotide cap comprises m⁷G_{3′OMe}pppCpU. In some embodiments, a trinucleotide cap comprises m⁷G_{3′OMe}pppGpA. In some embodiments, a trinucleotide cap comprises m⁷G_{3′OMe}pppGpC. In some embodiments, a trinucleotide cap comprises m⁷G_{3′OMe}pppGpG. In some embodiments, a trinucleotide cap comprises m⁷G_{3′OMe}pppGpU. In some embodiments, a trinucleotide cap comprises m⁷G_{3′OMe}pppUpA. In some embodiments, a trinucleotide cap comprises m⁷G_{3′OMe}pppUpC. In some embodiments, a trinucleotide cap comprises m⁷G_{3′OMe}pppUpG. In some embodiments, a trinucleotide cap comprises m⁷G_{3′OMe}pppUpU.

A trinucleotide cap, in other embodiments, comprises a sequence selected from the following sequences: m⁷G_{3′OMe}pppA_{2′OMe}pA, m⁷G_{3′OMe}pppA_{2′OMe}pC, m⁷G_{3′OMe}pppA_{2′OMe}pG, m⁷G_{3′OMe}pppA_{2′OMe}pU, m⁷G_{3′OMe}pppC_{2′OMe}pA, m⁷G_{3′OMe}pppC_{2′OMe}pC, m⁷G_{3′OMe}pppC_{2′OMe}pG, m⁷G_{3′OMe}pppC_{2′OMe}pU, m⁷G_{3′OMe}pppG_{2′OMe}pA, m⁷G_{3′OMe}pppG_{2′OMe}pC, m⁷G_{3′OMe}pppG_{2′OMe}pG, m⁷G_{3′OMe}pppG_{2′OMe}pU, m⁷G_{3′OMe}pppU_{2′OMe}pA, m⁷G_{3′OMe}pppU_{2′OMe}pC, m⁷G_{3′OMe}pppU_{2′OMe}pG, and m⁷G_{3′OMe}pppU_{2′OMe}pU.

In some embodiments, a trinucleotide cap comprises m⁷G_{3′OMe}pppA_{2′OMe}pA. In some embodiments, a trinucleotide cap comprises m⁷G_{2′OMe}pppA_{2′OMe}pC. In some embodiments, a trinucleotide cap comprises m⁷G_{3′OMe}pppA_{2′OMe}pG. In some embodiments, a trinucleotide cap comprises m⁷G_{3′OMe}pppA_{2′OMe}pU. In some embodiments, a trinucleotide cap comprises m⁷G_{3′OMe}pppC_{2′OMe}pA. In some embodiments, a trinucleotide cap comprises m⁷G_{3′OMe}pppC_{2′OMe}pC. In some embodiments, a trinucleotide cap comprises m⁷G_{3′OMe}pppC_{2′OMe}pG. In some embodiments, a trinucleotide cap comprises m⁷G_{3′OMe}pppC_{2′OMe}pU. In some embodiments, a trinucleotide cap comprises m⁷G_{3′OMe}pppG_{2′OMe}pA. In some embodiments, a trinucleotide cap comprises m⁷G_{3′OMe}pppG_{2′OMe}pC. In some embodiments, a trinucleotide cap comprises m⁷G_{3′OMe}pppG_{2′OMe}pG. In some embodiments, a trinucleotide cap comprises m′G_{3′OMe}pppG_{2′OMe}pU. In some embodiments, a trinucleotide cap comprises m⁷G_{3′OMe}pppU_{2′OMe}pA. In some embodiments, a trinucleotide cap comprises m⁷G_{3′OMe}pppU_{2′OMe}pC. In some embodiments, a trinucleotide cap comprises m⁷G_{3′OMe}pppU_{2′OMe}pG. In some embodiments, a trinucleotide cap comprises m⁷G_{3′OMe}pppU_{2′OMe}pU.

A trinucleotide cap, in still other embodiments, comprises a sequence selected from the following sequences: m⁷GpppA_{2′OMe}pA, m⁷GpppA_{2′OMe}pC, m⁷GpppA_{2′OMe}pG, m⁷GpppA_{2′OMe}pU, m⁷GpppC_{2′OMe}pA, m⁷GpppC_{2′OMe}pC, m⁷GpppC_{2′OMe}pG, m⁷GpppC_{2′OMe}pU, m⁷GpppG_{2′OMe}pA, m⁷GpppG_{2′OMe}pC, m⁷GpppG_{2′OMe}pG, m⁷GpppG_{2′OMe}pU, m⁷GpppU_{2′OMe}pA, m⁷GpppU_{2′OMe}pC, m⁷GpppU_{2′OMe}pG, and m⁷GpppU_{2′OMe}pU.

In some embodiments, a trinucleotide cap comprises m⁷GpppA_{2′OMe}pA. In some embodiments, a trinucleotide cap comprises m⁷GpppA_{2′OMe}pC. In some embodiments, a trinucleotide cap comprises m⁷GpppA_{2′OMe}pG. In some embodiments, a trinucleotide cap comprises m⁷GpppA_{2′OMe}pU. In some embodiments, a trinucleotide cap comprises m⁷GpppC_{2′OMe}pA. In some embodiments, a trinucleotide cap comprises m⁷GpppC_{2′OMe}pC. In some embodiments, a trinucleotide cap comprises m⁷GpppC_{2′OMe}pG. In some embodiments, a trinucleotide cap comprises m⁷GpppC_{2′OMe}pU. In some embodiments, a trinucleotide cap comprises m⁷GpppG_{2′OMe}pA. In some embodiments, a trinucleotide cap comprises m⁷GpppG_{2′OMe}pC. In some embodiments, a trinucleotide cap comprises m⁷GpppG_{2′OMe}pG. In some embodiments, a trinucleotide cap comprises m⁷GpppG_{2′OMe}pU. In some embodiments, a trinucleotide cap comprises m⁷GpppU_{2′OMe}pA. In some embodiments, a trinucleotide cap comprises m⁷Gppp_{2′OMe}pC. In some embodiments, a trinucleotide cap comprises m⁷Gppp_{2′OMe}pG. In some embodiments, a trinucleotide cap comprises m⁷GpppU_{2′OMe}pU. In some embodiments, a trinucleotide cap comprises m⁷Gpppm⁶A_{2′Ome}pG. In some embodiments, a trinucleotide cap comprises m⁷Gpppe⁶A_{2′Ome}pG.

In some embodiments, a trinucleotide cap comprises GAG. In some embodiments, a trinucleotide cap comprises GCG. In some embodiments, a trinucleotide cap comprises GUG. In some embodiments, a trinucleotide cap comprises GGG.

In some embodiments, a trinucleotide cap comprises any one of the following structures:

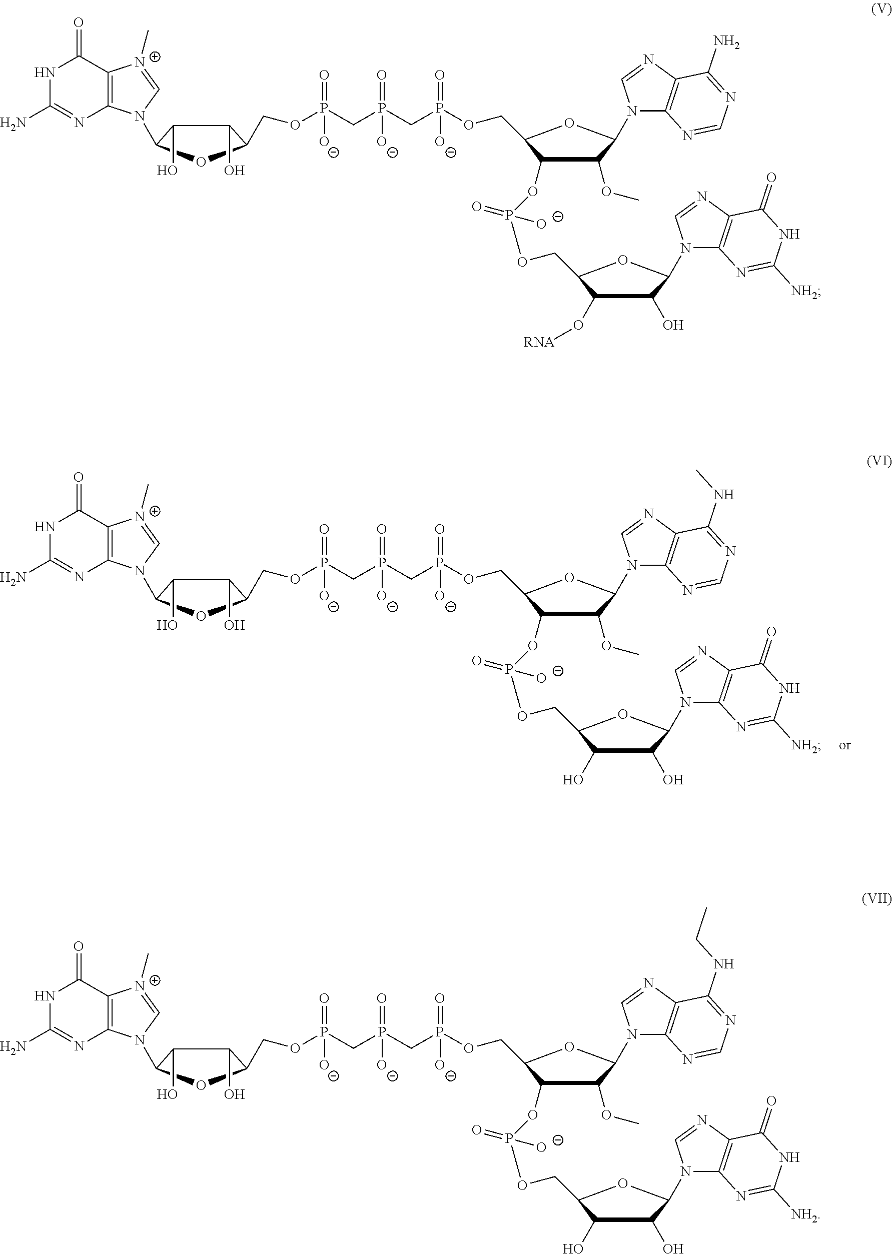

In some embodiments, the cap analog comprises a tetranucleotide cap. In some embodiments, the tetranucleotide cap comprises a trinucleotide as set forth above. In some embodiments, the tetranucleotide cap comprises ^{m7}GpppN₁N₂N₃, where N₁, N₂, and N₃ are optional (i.e., can be absent or one or more can be present) and are independently a natural, a modified, or an unnatural nucleoside base. In some embodiments, ^{m7}G is further methylated, e.g., at the 3′ position. In some embodiments, the ^{m7}G comprises an O-methyl at the 3′ position. In some embodiments N₁, N₂, and N₃ if present, optionally, are independently an adenine, a uracil, a guanidine, a thymine, or a cytosine. In some embodiments, one or more (or all) of N₁, N₂, and N₃, if present, are methylated, e.g., at the 2′ position. In some embodiments, one or more (or all) of N₁, N₂, and N₃, if present have an O-methyl at the 2′ position.

In some embodiments, the tetranucleotide cap comprises the following structure:

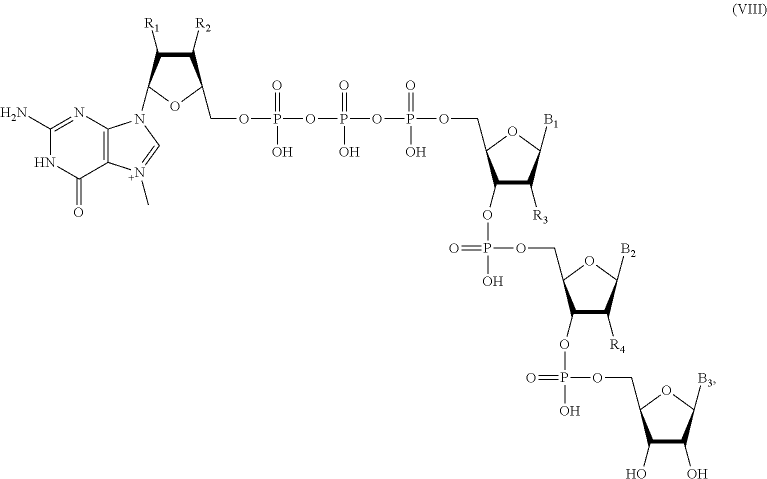

wherein B₁, B₂, and B₃ are independently a natural, a modified, or an unnatural nucleoside based; and R₁, R₂, R₃, and R₄ are independently OH or O-methyl. In some embodiments, R₃ is O-methyl and R₄ is OH. In some embodiments, R₃ and R₄ are O-methyl. In some embodiments, R₄ is O-methyl. In some embodiments, R₁ is OH, R₂ is OH, R₃ is O-methyl, and R₄ is OH. In some embodiments, R₁ is OH, R₂ is OH, R₃ is O-methyl, and R₄ is O-methyl. In some embodiments, at least one of R₁ and R₂ is O-methyl, R₃ is O-methyl, and R₄ is OH. In some embodiments, at least one of R₁ and R₂ is O-methyl, R₃ is O-methyl, and R₄ is O-methyl.

In some embodiments, B₁, B₃, and B₃ are natural nucleoside bases. In some embodiments, at least one of B₁, B₂, and B₃ is a modified or unnatural base. In some embodiments, at least one of B₁, B₂, and B₃ is N6-methyladenine. In some embodiments, B₁ is adenine, cytosine, thymine, or uracil. In some embodiments, B₁ is adenine, B₂ is uracil, and B₃ is adenine. In some embodiments, R₁ and R₂ are OH, R₃ and R₄ are O-methyl, B₁ is adenine, B₂ is uracil, and B₃ is adenine.

In some embodiments the tetranucleotide cap comprises a sequence selected from the following sequences: GAAA, GACA, GAGA, GAUA, GCAA, GCCA, GCGA, GCUA, GGAA, GGCA, GGGA, GGUA, GUCA, and GUUA. In some embodiments the tetranucleotide cap comprises a sequence selected from the following sequences: GAAG, GACG, GAGG, GAUG, GCAG, GCCG, GCGG, GCUG, GGAG, GGCG, GGGG, GGUG, GUCG, GUGG, and GUUG. In some embodiments the tetranucleotide cap comprises a sequence selected from the following sequences: GAAU, GACU, GAGU, GAUU, GCAU, GCCU, GCGU, GCUU, GGAU, GGCU, GGGU, GGUU, GUAU, GUCU, GUGU, and GUUU. In some embodiments the tetranucleotide cap comprises a sequence selected from the following sequences: GAAC, GACC, GAGC, GAUC, GCAC, GCCC, GCGC, GCUC, GGAC, GGCC, GGGC, GGUC, GUAC, GUCC, GUGC, and GUUC.

A tetranucleotide cap, in some embodiments, comprises a sequence selected from the following sequences: m⁷G_{3′OMe}pppApApN, m⁷G_{3′OMe}pppApCpN, m⁷G_{3′OMe}pppApGpN, m⁷G_{3′OMe}pppApUpN, m⁷G_{3′OMe}pppCpApN, m⁷G_{3′OMe}pppCpCpN, m⁷G_{3′OMe}pppCpGpN, m⁷GxoMepppCpUpN, m⁷G_{3′OMe}pppGpApN, m⁷G_{3′OMe}pppGpCpN, m⁷G_{3′OMe}pppGpGpN, m⁷G_{3′OMe}pppGpUpN, m⁷G_{3′OMe}pppUpApN, m⁷G_{3′OMe}pppUpCpN, m⁷G_{3′OMe}pppUpGpN, and m⁷G_{3′OMe}pppUpUpN, where N is a natural, a modified, or an unnatural nucleoside base.

A tetranucleotide cap, in other embodiments, comprises a sequence selected from the following sequences: m⁷G_{3′OMe}pppA_{2′OMe}pApN, m⁷G_{3′OMe}pppA_{2′OMe}pCpN, m⁷G_{3′OMe}pppA_{2′OMe}GpN, m⁷G_{3′OMe}pppA_{2′OMe}UpN, m⁷G_{3′OMe}pppC_{2′OMe}pApN, m⁷G_{3′OMe}pppC_{2′OMe}pCpN, m⁷G_{3′OMe}pppC_{2′OMe}pGpN, m⁷G_{3′OMe}pppC_{2′OMe}pUpN, m⁷G_{3′OMe}pppG_{2′OMe}pApN, m⁷G_{3′OME}pppG_{2′OMe}pCpN, m⁷G_{3′OMe}pppG_{2′OMe}pGpN, m⁷G_{3′OMe}pppG_{2′OMe}pUpN, m⁷G_{3′OMe}pppU_{2′OMe}pApN, m⁷G_{3′OMe}pppU_{2′OMe}pCpN, m⁷G_{3′OMe}pppU_{2′OMe}pGpN, and m⁷G_{3′OMe}pppU_{2′OMe}pUpN, where N is a natural, a modified, or an unnatural nucleoside base.

A tetranucleotide cap, in still other embodiments, comprises a sequence selected from the following sequences: m⁷GpppA_{2′OMe}pApN, m⁷GpppA_{2′OMe}pCpN, m⁷GpppA_{2′OMe}GpN, m⁷GpppA_{2′OMe}pUpN, m⁷GpppC_{2′OMe}pApN, m⁷GpppC_{2′OMe}pCpN, m⁷GpppC_{2′OMe}pGpN, m⁷GpppC_{2′OMe}pUpN, m⁷GpppG_{2′OMe}pApN, m⁷GpppG_{2′OMe}pCpN, m⁷GpppG_{2′OMe}pGpN, m⁷GpppG_{2′OMe}pUpN, m⁷GpppU_{2′OMe}pApN, m⁷GpppU_{2′OMe}pCpN, m⁷GpppU_{2′OMe}pGpN, and m⁷GpppU_{2′OMe}pUpN, where N is a natural, a modified, or an unnatural nucleoside base.

A tetranucleotide cap, in other embodiments, comprises a sequence selected from the following sequences: m⁷G_{3′OMe}pppA_{2′OMe}pA_{2′OMe}pN, m⁷G_{3′OMe}pppA_{2′OMe}pC_{2′OMe}pN, m⁷G_{3′OMe}pppC_{2′OMe}pC_{2′OMe}pN, m⁷G_{3′OMe}pppC_{2′OMe}pG_{2′OMe}pN, m⁷G_{3′OMe}pppC_{2′OMe}pU_{2′OMe}pN, m⁷G_{3′OMe}pppG_{2′OMe}pA_{2′OMe}pN, m⁷G_{3′OMe}pppG_{2′OMe}pC_{2′OMe}pN, m⁷G_{3′OMe}pppG_{2′OMe}pG_{2′OMe}pN, m⁷G_{3′OMe}pppG_{2′OMe}pU_{2′OMe}pN, m⁷G_{3′OMe}pppU_{2′OMe}pA_{2′OMe}pN, m⁷G_{3′OMe}pppU_{2′OMe}pC_{2′OMe}pN, m⁷G_{3′OMe}pppU_{2′OMe}pG_{2′OMe}pN, and m⁷G_{3′OMe}pppU_{2′OMe}pU_{2′OMe}pN, where N is a natural, a modified, or an unnatural nucleoside base.

A tetranucleotide cap, in still other embodiments, comprises a sequence selected from the following sequences: m⁷GpppA_{2′OMe}pA_{2′OMe}pN, m⁷GpppA_{2′OMe}pC_{2′OMe}pN, m⁷GpppA_{2′OMe}pG_{2′OMe}pN, m⁷GpppA_{2′OMe}pU_{2′OMe}pN, m⁷GpppC_{2′OMe}pA_{2′OMe}pN, m⁷GpppC_{2′OMe}pC_{2′OMe}pN, m⁷GpppC_{2′OMe}pG_{2′OMe}pN, m⁷GpppC_{2′OMe}pU_{2′OMe}pN, m⁷GpppG_{2′OMe}pA_{2′OMe}pN, m⁷GpppG_{2′OMe}C_{2′OMe}pN, m⁷GpppG_{2′OMe}pG_{2′OMe}pN, m⁷GpppG_{2′OMe}pU_{2′OMe}pN, m⁷GpppU_{2′OMe}pA_{2′OMe}pN, m⁷Gppp_{2′OMe}pC_{2′OMe}pN, m⁷Gppp_{2′OMe}pG_{2′OMe}pN, and m⁷GpppU_{2′OMe}pU_{2′OMe}pN, where N is a natural, a modified, or an unnatural nucleoside base.

In some embodiments, a tetranucleotide cap comprises GGAG. In some embodiments, a tetranucleotide cap comprises the following structure:

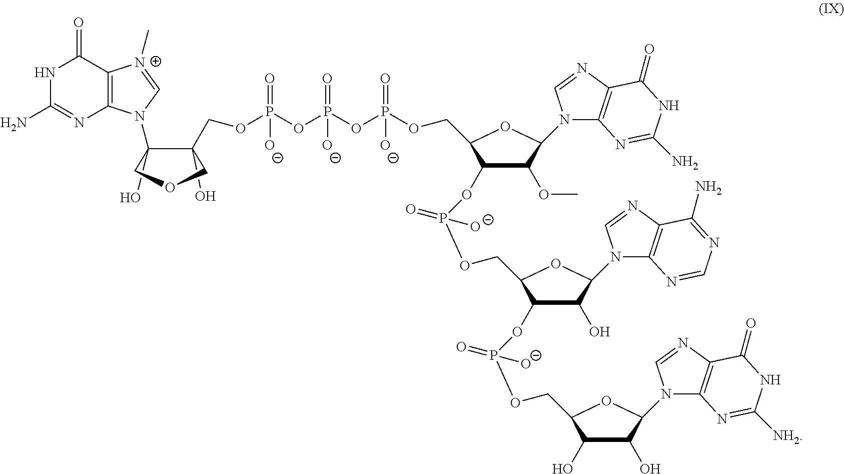

The capping efficiency of a post-transcriptional or co-transcriptional capping reaction may vary. As used herein “capping efficiency” refers to the amount (e.g., expressed as a percentage) of mRNAs comprising a cap structure relative to the total mRNAs in a mixture (e.g., a post-translational capping reaction or a co-transcriptional calling reaction). In some embodiments, the capping efficiency of a capping reaction is at least 60%, 70%, 80%, 90%, 95%, 99%, or 99.9% (e.g., after the capping reaction at least 60%, 70%, 80%, 90%, 95%, 99%, or 99.9% of the input mRNAs comprise a cap). In some embodiments, multivalent co-IVT reactions described herein do not affect the capping efficiency of the mRNAs resulting from the IVT reaction.

A 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, a composition comprises an RNA (e.g., mRNA) having an ORF that encodes a signal peptide fused to the expressed polypeptide. Signal peptides, usually comprising the N-terminal 15-60 amino acids of proteins, are typically needed for the translocation across the membrane on the secretory pathway and, thus, universally control the entry of most proteins both in eukaryotes and prokaryotes to the secretory pathway. A signal peptide may have a length of 15-60 amino acids.

In some embodiments, an ORF encoding a polypeptide is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, an RNA (e.g., mRNA) is not chemically modified and comprises the standard ribonucleotides consisting of adenosine, guanosine, cytosine and uridine. In some embodiments, nucleotides and nucleosides comprise standard nucleoside residues such as those present in transcribed RNA (e.g. A, G, C, or U). In some embodiments, nucleotides and nucleosides comprise standard deoxyribonucleosides such as those present in DNA (e.g. dA, dG, dC, or dT).

The compositions can comprise, in some embodiments, an RNA having an open reading frame encoding a polypeptide, wherein the nucleic acid comprises nucleotides and/or nucleosides that can be standard (unmodified) or modified as is known in the art. In some embodiments, nucleotides and nucleosides comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide is one as is generally known or recognized in the art. Non-limiting examples of such naturally occurring modified nucleotides and nucleotides can be found, inter alia, in the widely recognized MODOMICS database.

Also provided are modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

In some embodiments, modified nucleobases in nucleic acids (e.g., RNA nucleic acids, such as mRNA nucleic acids) comprise 1-methyl-pseudouridine (m1ω), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a mRNA comprises 1-methyl-pseudouridine (m1ψ) substitutions at one or more or all uridine positions of the nucleic acid.

In some embodiments, a mRNA comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a mRNA comprises pseudouridine (ψ) substitutions at one or more or all uridine positions of the nucleic acid.

In some embodiments, a mRNA pseudouridine (ψ) substitutions at one or more or all uridine positions of the nucleic acid and 5-methyl cytidine substitutions at one or more or all cytidine positions of the nucleic acid.

In some embodiments, a mRNA comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids may be partially or fully modified along the entire length of the molecule. For example, one or more or all or a given type of nucleotide (e.g., purine or pyrimidine, or any one or more or all of A, G, U, C) may be uniformly modified in a nucleic acid, or in a predetermined sequence region thereof (e.g., in the mRNA including or excluding the poly(A) tail). In some embodiments, all nucleotides X in a nucleic acid (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The mRNAs may comprise one or more regions or parts which act or function as an untranslated region. Where mRNAs are designed to encode at least one polypeptide of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas the 3′ UTR starts immediately following the stop codon and continues until the transcriptional termination signal. The regulatory features of a UTR can be incorporated into the polynucleotides to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

According to some aspects, a stabilizing compound (e.g., a compound of Formula I, of Formula II, or a tautomer or solvate thereof) interacts with a nucleic acid. In some embodiments, the compound interacts with a nucleic acid comprised within a lipid nanostructure (e.g., a lipid nanoparticle, liposome, or lipoplex) disclosed herein. In some embodiments, the compound interacts with a nucleic acid via electrostatic binding. In some embodiments, the compound intercalates with a nucleic acid. In some embodiments, the compound intercalates with a nucleic acid comprised within a lipid nanostructure. In some embodiments, the compound binds with a nucleic acid. In some embodiments, the compound reversibly binds with a nucleic acid. In some embodiments, the compound binds with a nucleic acid comprised within a lipid nanostructure.

In some embodiments, a stabilizing compound (e.g., a compound of Formula I, of Formula II, or a tautomer or solvate thereof) binds with a nucleic acid externally. In some embodiments, a stabilizing compound interacts with a nucleic acid via pi-pi stacking. In some embodiments, a stabilizing compound interacts with the bases a nucleic acid via pi-pi stacking. In some embodiments, a stabilizing compound interacts with a nucleic acid and changes backbone helicity of the nucleic acid. In some embodiments, a stabilizing compound self-associates. In some embodiments, a stabilizing compound self-associates via pi-pi stacking.

In some embodiments, a stabilizing compound (e.g., a compound of Formula I, of Formula II, or a tautomer or solvate thereof) has a similar proportion of nucleic acid contacts to self-contacts. In some embodiments, a stabilizing compound has a higher proportion of self-contacts to nucleic acid contacts. In some embodiments, a stabilizing compound binds to nucleic acid ribose contacts or to nucleic acid base contacts. In some embodiments, a stabilizing compound self-associates, binds to nucleic acid ribose contacts, or binds to nucleic acid base contacts. In some embodiments, a stabilizing compound self-associates, binds to nucleic acid ribose contacts, or binds to nucleic acid base contacts preferentially over binding to nucleic acid phosphate contacts. In some embodiments, a stabilizing compound does not substantially bind to nucleic acid phosphate contacts.

In some embodiments, a stabilizing compound (e.g., a compound of Formula I, of Formula II, or a tautomer or solvate thereof) is positively charged. In some embodiments, the positive charge contributes to nucleic acid binding.

In some embodiments, a stabilizing compound (e.g., a compound of Formula I, of Formula II, or a tautomer or solvate thereof) interacts with a nucleic acid and provides shielding from solvent. In some embodiments, a stabilizing compound shields ribose from water. In some embodiments, a stabilizing compound shields ribose from water more than the compound shields the phosphate groups of a nucleic acid. In some embodiments, a stabilizing compound reduces solvent exposure of ribose. In some embodiments, a stabilizing compound reduces solvent exposure of phosphate groups of a nucleic acid. In some embodiments, a stabilizing compound reduces solvent exposure of ribose more than it reduces the solvent exposure of phosphate groups of a nucleic acid. In some embodiments, the solvent exposure is measured by the solvent accessible surface area (SASA). In some embodiments, a stabilizing compound decreases the solvent accessible area of ribose to about 5-10 nm². In some embodiments, a stabilizing compound decreases the solvent accessible area of ribose to about 6-8 nm². In some embodiments, a stabilizing compound decreases the solvent accessible area of phosphate to about 9-12 nm². In some embodiments, a stabilizing compound decreases the solvent accessible area of phosphate to about 10-11 nm².

In some embodiments, a stabilizing compound (e.g., a compound of Formula I, of Formula II, or a tautomer or solvate thereof) interacts with a nucleic acid (e.g., an mRNA) with a binding affinity defined by a particular equilibrium dissociation constant. In some embodiments, the equilibrium dissociation constant is less than 10⁻³ M (e.g., less than 10⁻⁴ M, less than 10⁻⁵ M, less than 10⁻⁶ M, less than 10⁻⁷ M, less than 10⁻⁸ M, or less than 10⁻⁹ M). In some embodiments, the equilibrium dissociation constant is between 10⁻³ M and 10⁻⁴ M, between 10⁻³ M and 10⁻⁵ M, between 10⁻³ M and 10⁻⁶ M, between 10⁻³ M and 10⁻⁷ M, between 10⁻³ M and 10⁻⁸ M, between 10⁻³ M and 10⁻⁹ M, between 10⁻³ M and 10⁻¹⁰ M, between 10⁻⁴ M and 10⁻⁵ M, between 10⁻⁴ M and 10⁻⁶ M, between 10⁻⁴ M and 10⁻⁷ M, between 10⁻⁴ M and 10⁻⁸ M, between 10⁻⁴ M and 10⁻⁹ M, between 10⁻⁴ M and 10⁻¹⁰ M, between 10⁻⁵ M and 10⁻⁶ M, between 10⁻⁵ M and 10⁻⁷ M, between 10⁻⁵ M and 10⁻⁸ M, between 10⁻⁵ M and 10⁻⁹ M, between 10⁻⁵ M and 10⁻¹⁰ M, between 10⁻⁶ M and 10⁻⁷ M, between 106 M and 10⁻⁸ M, between 10⁻⁶ M and 10⁻⁹ M, between 10⁻⁶ M and 10⁻¹⁰ M, between 10⁻⁷ M and 10⁻⁸ M, between 10⁻⁷ M and 10⁻⁹ M, between 10⁻⁷ M and 10⁻¹⁰ M, between 10⁻⁸ M and 10⁻⁹ M, between 10-% M and 10⁻¹⁰ M, or between 10⁻⁹ M and 10⁻¹⁰ M. In some embodiments, the equilibrium dissociation constant is between 10⁻³ M and 10⁻⁴ M or between 10⁻³ M and 10⁻⁵ M. In certain embodiments, the equilibrium dissociation constant is about 10⁻⁵ M. In certain embodiments, the equilibrium dissociation constant is about 10⁻⁶ M.

mRNA molecules that are conformationally stabilized by compounds provided herein can exhibit thermal unfolding temperatures (measured by circular dichroism or DSC, for example) that are higher than in the absence of such stabilizing compound. See, e.g., FIG. XX. In some embodiments, a stabilizing compound (e.g., a compound of Formula I, of Formula II, or a tautomer or solvate thereof) confers increased stability to a nucleic acid (e.g., an mRNA) in a folded structure. In some embodiments, a stabilizing compound confers increased stability to a folded structure of a nucleic acid (e.g., an mRNA) relative to its unfolded or less folded (i.e., more linear) form. Changes in stability of a folded structure of a nucleic acid can be identified by one of ordinary skill in the art, for example, by circular dichroism. Such changes in stability of a folded structure may, for example, result in changes in the amplitude of peaks in circular dichroism spectra. In some embodiments, a stabilizing compound enhances the thermal stability of a nucleic acid (e.g., an mRNA) in a folded state. Changes in thermal stability of a folded state of a nucleic acid can be identified by one of ordinary skill in the art, for example, by differential scanning calorimetry. Such changes in thermal stability may, for example, result in shifts of differential scanning calorimetry thermograms.

In some embodiments, a stabilizing compound (e.g., a compound of Formula I, of Formula II, or a tautomer or solvate thereof) causes compaction of a nucleic acid molecule (e.g., an mRNA) upon interaction with the nucleic acid molecule. In some embodiments, a stabilizing compound causes a decrease in the hydrodynamic radius of a nucleic acid molecule (e.g., an mRNA) upon interaction with the nucleic acid molecule. In some embodiments, a stabilizing compound causes compaction or a decrease in the hydrodynamic radius of a nucleic acid molecule by 5%, 10%, 15%, 20%, 25%, 30%, 35%, 40%, 45%, 50%, 55%, 60%, or more. In some embodiments, a stabilizing compound causes compaction or a decrease in the hydrodynamic radius of a nucleic acid molecule when the compound is in a concentration of 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 60 μM, 70 μM, 80 μM, 90 μM, or 100 μM. In some embodiments, a stabilizing compound causes compaction or a decrease in the hydrodynamic radius of a nucleic acid molecule when the compound is in a concentration of 10 μM. In some embodiments, a stabilizing compound causes compaction of a nucleic acid molecule (e.g., an mRNA) within a lipid nanostructure (e.g., a lipid nanoparticle, liposome, or lipoplex). In some embodiments, a stabilizing compound causes compaction of a nucleic acid molecule within a lipid nanostructure without changing the size of the lipid nanostructure. Compaction of a nucleic acid molecule or a decrease in its hydrodynamic radius can be measured by one of ordinary skill in the art, for example, via dynamic light scattering or transmission electron microscopy measurements. However, such compaction cannot be directly measured within a lipid nanoparticle.

### Lipid Nanoparticle Formulations

A lipid nanoparticle (LNP) refers to a nanoscale construct (e.g., a nanoparticle, typically less than 100 nm in diameter) comprising lipid molecules arranged in a substantially spherical (e.g., spheroid) geometry, sometimes encapsulating one or more additional molecular species. In some embodiments, the LNP contains a bleb region, e.g., as described in Brader et al., Biophysical Journal 120: 1-5 (2021). A LNP may comprise or one or more types of lipids, including but not limited to amino lipids (e.g., ionizable amino lipids), neutral lipids, non-cationic lipids, charged lipids, PEG-modified lipids, phospholipids, structural lipids and sterols. In some embodiments, a LNP may further comprise one or more cargo molecules, including but not limited to nucleic acids (e.g., mRNA, plasmid DNA, DNA or RNA oligonucleotides, siRNA, shRNA, snRNA, snoRNA, lncRNA, etc.), small molecules, proteins and peptides. A LNP may have a unilamellar structure (i.e., having a single lipid layer or lipid bilayer surrounding a central region) or a multilamellar structure (i.e., having more than one lipid layer or lipid bilayer surrounding a central region). In some embodiments, a lipid nanoparticle may be a liposome. A liposome is a nanoparticle comprising lipids arranged into one or more concentric lipid bilayers around a central region. The central region of a liposome may comprises an aqueous solution, suspension, or other aqueous composition.

In some embodiments, nucleic acids are formulated as lipid nanoparticle (LNP) compositions. Lipid nanoparticles typically comprise amino lipid, phospholipid, structural lipid and PEG lipid components along with the nucleic acid cargo of interest. The lipid nanoparticles provided herein can be generated using components, compositions, and methods as are generally known in the art, see for example PCT/US2016/052352, PCT/US2016/068300; PCT/US2017/037551; PCT/US2015/027400; PCT/US2016/047406; PCT/US2016/000129; PCT/US2016/014280; PCT/US2017/038426; PCT/US2014/027077; PCT/US2014/055394; PCT/US2016/052117; PCT/US2012/069610; PCT/US2017/027492; PCT/US2016/059575; PCT/US2016/069491; PCT/US2016/069493; and PCT/US2014/066242, all of which are incorporated by reference herein in their entirety.

In some embodiments, a LNP comprises an N:P ratio of from about 2:1 to about 30:1.

In some embodiments, a LNP comprises an N:P ratio of about 6:1.

In some embodiments, a LNP comprises an N:P ratio of about 3:1, 4:1, or 5:1.

In some embodiments, a LNP comprises a wt/wt ratio of the ionizable amino lipid component to the RNA of from about 10:1 to about 100:1.

In some embodiments, a LNP comprises a wt/wt ratio of the ionizable amino lipid component to the RNA of about 20:1.

In some embodiments, a LNP comprises a wt/wt ratio of the ionizable amino lipid component to the RNA of about 10:1.

In some embodiments, a LNP has a mean diameter from about 30 nm to about 150 nm.

In some embodiments, a LNP has a mean diameter from about 60 nm to about 120 nm.

In some embodiments, the lipid nanoparticle has a diameter of at most 80 nm, at most 70 nm, at most 60 nm, at most 50 nm, at most 40 nm, at most 30 nm, or at most 20 nm.

In some embodiments, the lipid nanoparticle has a diameter of at most 30 nm.

In some embodiments, the lipid nanoparticle has a diameter of at most 20 nm.

In some embodiments, the LNPs have one or more regions having a lamellar structure. In some embodiments, the lamellar structure diminishes with increasing temperature in the absence of stabilizer. In some embodiments, the LNP size increases with increasing time in the absence of stabilizer. In some embodiments, the LNP size increases with diminished lamellar structure in the absence of stabilizer. In some embodiments, loss of lamellar structure is not reversible in the absence of stabilizer. In some embodiments, loss of lamellar structure is not reversible at higher temperatures (e.g., 40° C.) in the absence of stabilizer.

In some embodiments, the lamellar structure diminishes with increasing temperature in the presence of stabilizer. In some embodiments, the lamellar structure is preserved at low temperature in the presence of stabilizer. In some embodiments, the lamellar structure is preserved at 5° C. in the presence of stabilizer.

In some embodiments, a lipid nanoparticle may comprise two or more components (e.g., amino lipid and nucleic acid, PEG-lipid, phospholipid, structural lipid). For instance, a lipid nanoparticle may comprise an amino lipid and a nucleic acid. Compositions comprising the lipid nanoparticles may be used for a wide variety of applications, including the stealth delivery of therapeutic payloads with minimal adverse innate immune response.

### Ionizable Amino Lipids

In some embodiments, a LNP provided herein may include one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those provided above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2.250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 21%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LC-MS), nuclear magnetic resonance (NMR), or mass spectrometry (MS). Those of ordinary skill in the art would be knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable amino lipid.

In some embodiments, the lipid nanoparticle comprises 45-55 mole percent (mol %) ionizable amino lipid. For example, lipid nanoparticle may comprise 45, 46, 47, 48, 49, 50, 51, 52, 53, 54, or 55 mol % ionizable amino lipid.

In some embodiments, the ionizable amino lipid is a compound of Formula (AI):

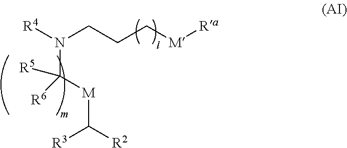

or its N-oxide, or a salt or isomer thereof,
wherein R′^{a} is R′^{branched}; wherein
R′^{branched} is:

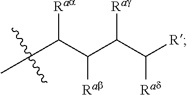

wherein

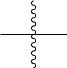

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

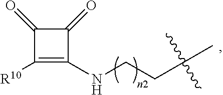

wherein

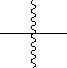

denotes a point of attachment; wherein R¹⁰ is N(R)₂; each R is independently selected from the group consisting of C₁₋₆ alkyl, C₂₋₃ alkenyl, and H; and
n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of the compounds of Formula (AI), R′^{a} is R′^{branched}; R′^{branched} is

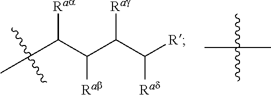

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched}; R′^{branched} is

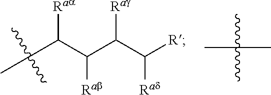

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of the compounds of Formula (AI), R′^{a} is R′^{branched}; R′^{branched} is

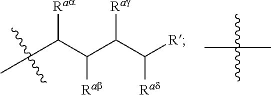

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

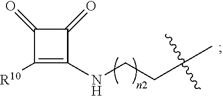

R¹⁰ NH(C₁₋₆ alkyl); n2 is 2; R⁵ is H; each R⁶ is H; M and M′ are each —C(O)O—; R′ is a C₁₋₁₂ alkyl; l is 5; and m is 7.

In some embodiments of the compounds of Formula (I), R′^{a} is R′^{branched}; R′^{branched} is

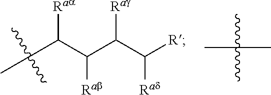

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the compound of Formula (I) is selected from:

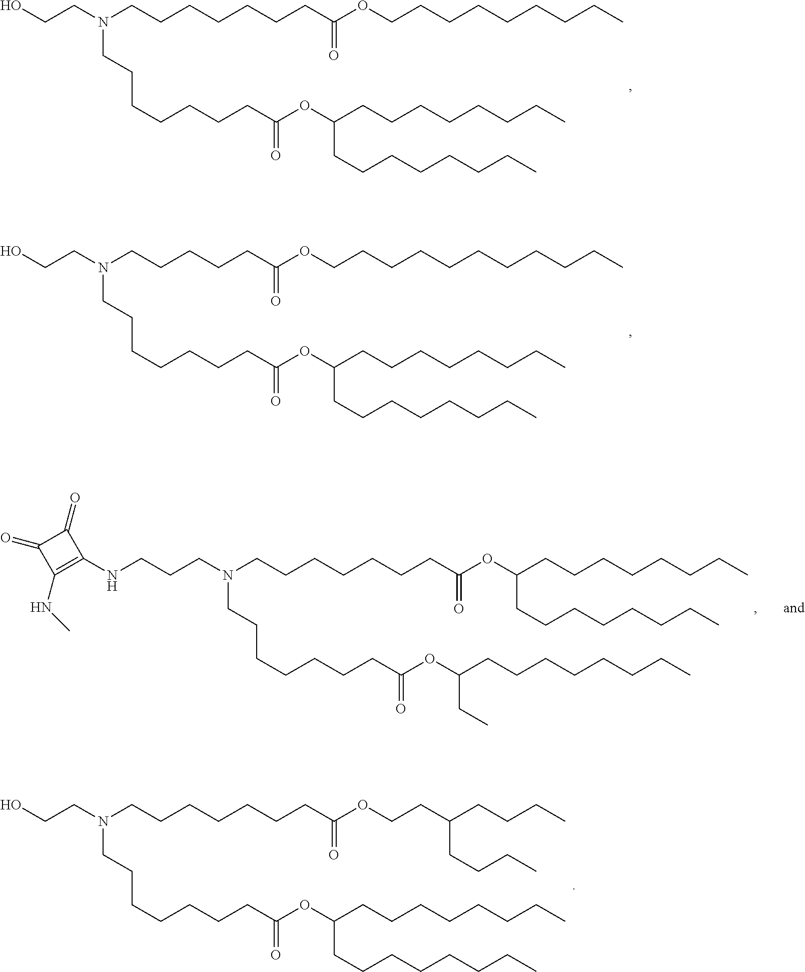 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the ionizable amino lipid is a compound of Formula (Ala):

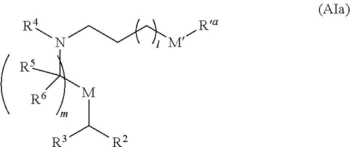

or its N-oxide, or a salt or isomer thereof,
wherein R′^{a} is R′^{branched}; wherein
R′^{branched} is:

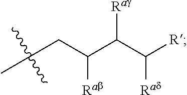

wherein

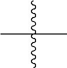

denotes a point of attachment; wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

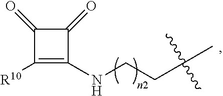

wherein

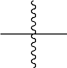

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the ionizable amino lipid is a compound of Formula (AIb):

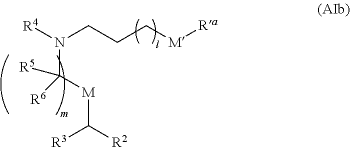

or its N-oxide, or a salt or isomer thereof,
wherein R′^{a} is R′^{branched}; wherein
R′^{branched} is:

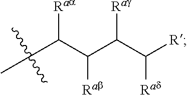

wherein

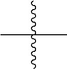

denotes a point of attachment; wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of Formula (AI) or (AIb), R′^{a} is R′^{branched}; R′^{branched} is

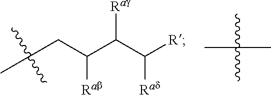

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of Formula (AI) or (AIb), R′^{a} is R′^{branched}; R′^{branched} is

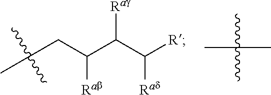

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of Formula (AI) or (AIb), R′^{a} is R′^{branched}; R′^{branched} is

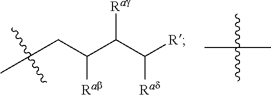

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments, the ionizable amino lipid is a compound of Formula (AIc):

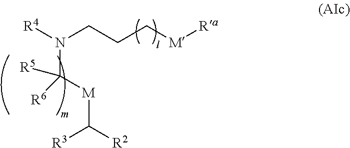

or its N-oxide, or a salt or isomer thereof,
wherein R′^{a} is R′^{branched}; wherein
R′^{branched} is:

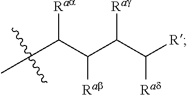

wherein

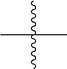

denotes a point of attachment;
   wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is

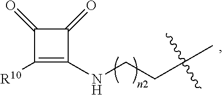

wherein

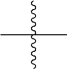

denotes a point of attachment; wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, R′^{a} is R′^{branched}; R′^{branched} is

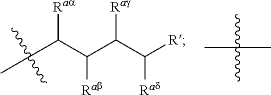

denotes a point of attachment; R^{aβ}, R^{aγ}, and R^{aδ} are each H; R^{aα} is C₂₋₁₂ alkyl; R² and R³ are each C₁₋₁₄ alkyl; R⁴ is

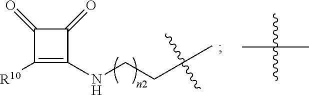

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; 1 is 5; and m is 7.

In some embodiments, the compound of Formula (AIc) is:

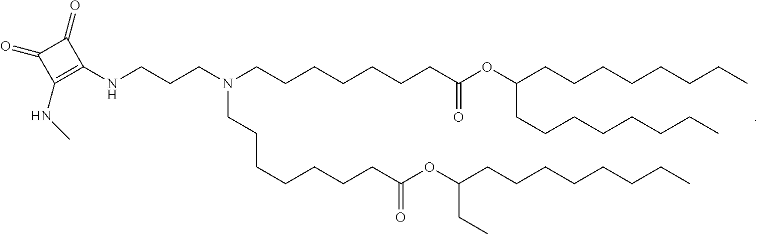 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the ionizable amino lipid is a compound of Formula (AII):

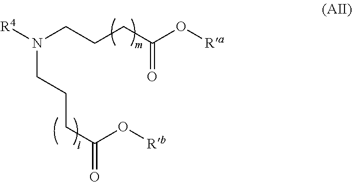

or its N-oxide, or a salt or isomer thereof,
wherein R′^{a} is R′^{branched} or R′^{cyclic}, wherein
R′^{branched} is:

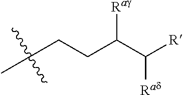

and R′^{cyclic} is:

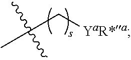

and
R′^{b} is:

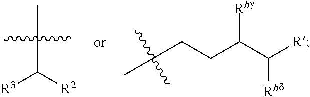

wherein

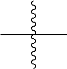

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

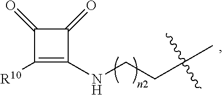

wherein

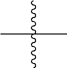

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;

Y^{a} is a C₃₋₆ carbocycle;

R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-a):

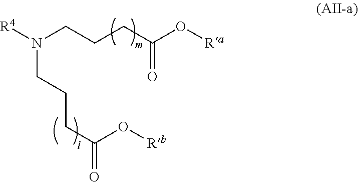

or its N-oxide, or a salt or isomer thereof,
wherein R′^{a} is R′^{branched} or R′^{cyclic}; wherein
R′^{branched} is:

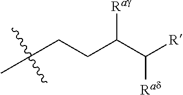

and R′^{b} is:

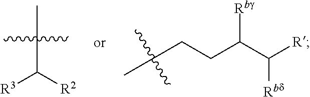

wherein

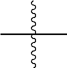

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

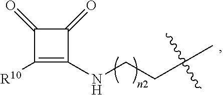

wherein

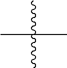

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-b):

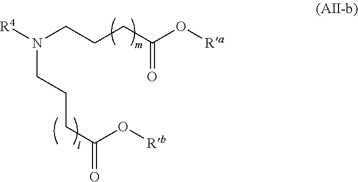

or its N-oxide, or a salt or isomer thereof,
wherein R′^{a} is R′^{branched} or R′^{cyclic}; wherein
R′^{branched} is:

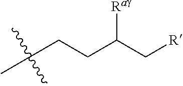

and R′b is:

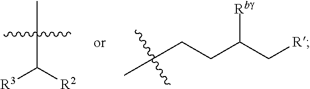

wherein

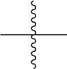

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

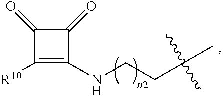

wherein

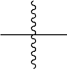

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-c):

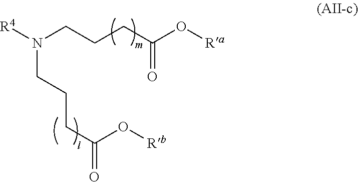

or its N-oxide, or a salt or isomer thereof,
wherein R′^{a} is R′^{branched} or R′^{cyclic}; wherein
R′^{branched} is:

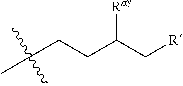

and R′^{b} is:

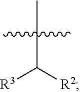

wherein

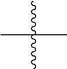

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

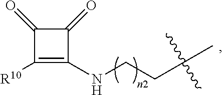

wherein

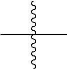

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-d):

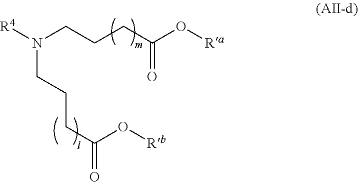

or its N-oxide, or a salt or isomer thereof,
wherein R′^{a} is R′^{branched} or R′^{cyclic}, wherein
R′^{branched} is:

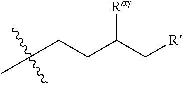

and R′^{b} is:

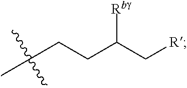

wherein

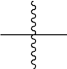

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙ OH wherein n is selected from the group consisting of 1, 2, 3, 4, and 5, and

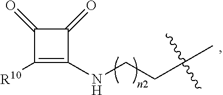

wherein

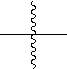

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-e):

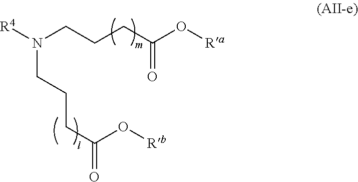

or its N-oxide, or a salt or isomer thereof,
wherein R′^{a} is R′^{branched} or R′^{cyclic}; wherein
R′^{branched} is:

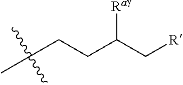

and R′^{b} is:

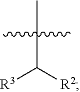

wherein

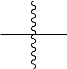

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (AII), (AII-a), (AII-b), (AII-c), (AII-d), or (AII-e), m and l are each independently selected from 4, 5, and 6. In some embodiments of the compound of Formula (AII), (AII-a), (AII-b), (AII-c), (AII-d), or (AII-e), m and l are each 5.

In some embodiments of the compound of Formula (AII), (AII-a), (AII-b), (AII-c), (AII-d), or (AII-e), each R′ independently is a C₁₋₁₂ alkyl. In some embodiments of the compound of Formula (AII), (AII-a), (AII-b), (AII-c), (AII-d), or (AII-e), each R′ independently is a C₂₋₅ alkyl.

In some embodiments of the compound of Formula (AII), (AII-a), (AII-b), (AII-c), (AII-d), or (AII-e), R′^{b} is:

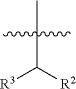

and R² and R³ are each independently a C₁₋₁₄ alkyl. In some embodiments of the compound of Formula (AII), (AII-a), (AII-b), (AII-c), (AII-d), or (AII-e), R′^{b} is:

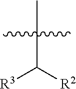

and R² and R³ are each independently a C₆₋₁₀ alkyl. In some embodiments of the compound of Formula (AII), (AII-a), (AII-b), (AII-c), (AII-d), or (AII-e), R′ is:

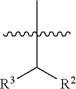

and R² and R³ are each a C₈ alkyl.

In some embodiments of the compound of Formula (AII), (AII-a), (AII-b), (AII-c), (AII-d), or (AII-e), R′^{branched} is:

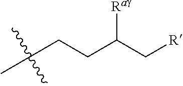

### and R′^{b} is:

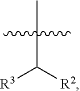

R^{aγ} is a C₁₋₁₂ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

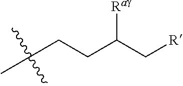

### and R′^{b} is:

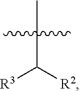

R^{aγ} is a C₂₋₆ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

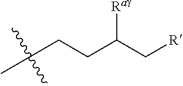

### and R′^{b} is:

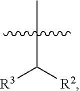

R^{aγ} is a C₂₋₆ alkyl, and R² and R³ are each a C₈ alkyl.

In some embodiments of the compound of Formula (AII), (AII-a), (AII-b), (AII-c), (AII-d), or (AII-e), R′^{branched} is:

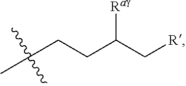

### R′^{b} is:

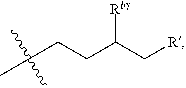

and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

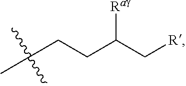

### R′^{b} is:

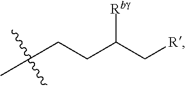

and R^{aγ} and R^{bγ} are each a C₂₋₆ alkyl.

In some embodiments of the compound of Formula (AII), (AII-a), (AII-b), (AII-c), (AII-d), or (AII-e), m and l are each independently selected from 4, 5, and 6 and each R′ independently is a C₁₋₁₂ alkyl. In some embodiments of the compound of Formula (AII), (AII-a), (AII-b), (AII-c), (AII-d), or (AII-e), m and l are each 5 and each R′ independently is a C₂₋₅ alkyl.

In some embodiments of the compound of (AII), (AII-a), (AII-b), (AII-c), (AII-d), or (AII-e), R′^{branched} is:

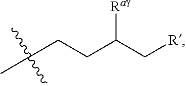

### R′^{b} is:

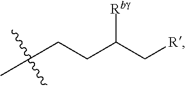

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

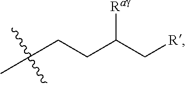

### R′^{b} is:

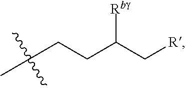

m and l are each 5, each R′ independently is a C₂₋₅ alkyl, and R^{aγ} and R^{bγ} are each a C₂₋₆ alkyl.

In some embodiments of the compound of Formula (AII), (AII-a), (AII-b), (AII-c), (AII-d), or (AII-e), R′^{branched} is:

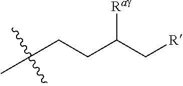

### and R′^{b} is:

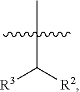

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl.

In some embodiments of the compound of Formula (AII), (AII-a), (AII-b), (AII-c), (AII-d), or (AII-e), R′^{branched} is:

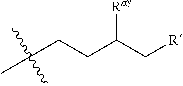

### and R′^{b} is:

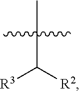

m and l are each 5, R′ is a C₂₋₅ alkyl, R^{aγ} is a C₂₋₆ alkyl, and R² and R³ are each a C₈ alkyl.

In some embodiments of the compound of (AII), (AII-a), (AII-b), (AII-c), (AII-d), or (AII-e), R⁴ is

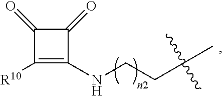

wherein R¹⁰ is NH(C₁₋₆ alkyl) and n2 is 2. In some embodiments of the compound of Formula (AII), (AII-a), (AII-b), (AII-c), (AII-d), or (AII-e), R⁴ is

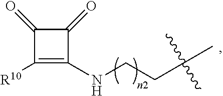

wherein R¹⁰ is NH(CH₃) and n2 is 2.

In some embodiments of the compound of Formula (AII), (AII-a), (AII-b), (AII-c), (AII-d), or (AII-e), R′^{branched} is:

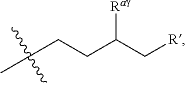

### R′^{b} is:

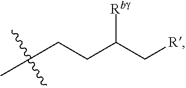

m and l are each independently selected from 4, 5, and 6, each R′ independently is a C₁₋₁₂ alkyl, R^{aγ} and R^{bγ} are each a C₁₋₁₂ alkyl, and R⁴ is

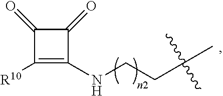

wherein R¹⁰ is NH(C₁₋₆ alkyl), and n2 is 2. In some embodiments of the compound of Formula (AII), (AII-a), (AII-b), (AII-c), (AII-d), or (AII-e), R′^{branched} is:

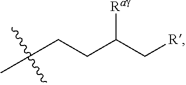

### R′^{b} is:

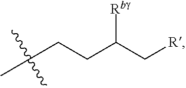

m and l are each 5, each R′ independently is a C₂₋₅ alkyl, R^{aγ} and R^{bγ} are each a C₂₋₆ alkyl, and R⁴ is

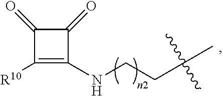

wherein R¹⁰ is NH(CH₃) and n2 is 2.

In some embodiments of the compound of Formula (AII), (AII-a), (AII-b), (AII-c), (AII-d), or (AII-e), R′^{branched} is:

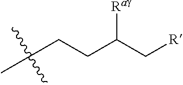

### and R′^{b} is:

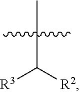

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

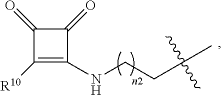

wherein R¹⁰ is NH(C₁₋₆ alkyl) and n2 is 2. In some embodiments of the compound of Formula (AII), (AII-a), (AII-b), (AII-c), (AII-d), or (AII-e), R′^{branched} is:

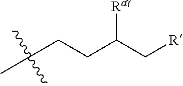

### and R′^{b} is:

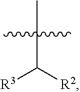

m and l are each 5, R′ is a C₂₋₅ alkyl, R^{aγ} is a C₂₋₆ alkyl, R² and R³ are each a C₈ alkyl, and R⁴ is

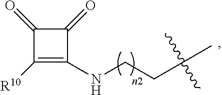

wherein R¹⁰ is NH(CH₃) and n2 is 2.

In some embodiments of the compound of Formula (AII), (AII-a), (AII-b), (AII-c), (AII-d), or (AII-e), R⁴ is —(CH₂)ₙOH and n is 2, 3, or 4. In some embodiments of the compound of Formula (AII), (AII-a), (AII-b), (AII-c), (AII-d), or (AII-e), R⁴ is —(CH₂)ₙOH and n is 2.

In some embodiments of the compound of Formula (AII), (AII-a), (AII-b), (AII-c), (AII-d), or (AII-e), R′^{branched} is:

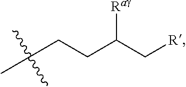

### R′^{b} is:

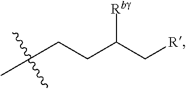

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (AII), (AII-a), (AII-b), (AII-c), (AII-d), or (AII-e), R′^{branched} is:

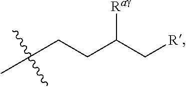

### R′^{b} is:

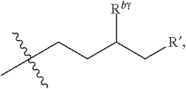

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable amino lipid is a compound of Formula (AII-f):

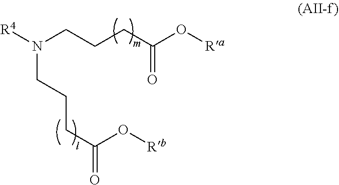

or its N-oxide, or a salt or isomer thereof,
wherein R′^{a} is R′^{branched} or R′^{cyclic}, wherein
R′^{branched} is:

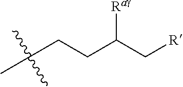

and R′^{b} is:

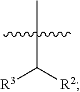

wherein

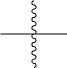

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (AII-f), m and l are each 5, and n is 2, 3, or 4.

In some embodiments of the compound of Formula (AII-f) R′ is a C₂₋₅ alkyl, R^{aγ} is a C₂₋₆ alkyl, and R² and R³ are each a C₆₋₁₀ alkyl.

In some embodiments of the compound of Formula (AII-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some embodiments, the ionizable amino lipid is a compound of Formula (AII-g):

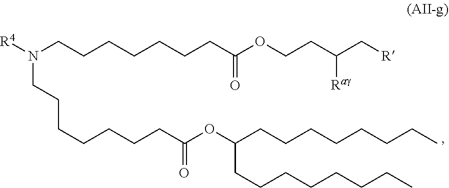

wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

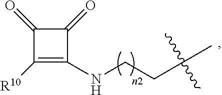

wherein

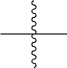

denotes a point of attachment, R¹⁰ is NH(C₁₋₆ alkyl), and n2 is selected from the group consisting of 1, 2, and 3. In some embodiments, the ionizable amino lipid is a compound of Formula (AII-h):

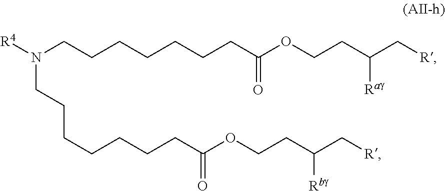

wherein
R^{aγ} and R^{bγ} are each independently a C₂₋₆ alkyl;
each R′ independently is a C₂₋₅ alkyl; and
R⁴ is selected from the group consisting of —(CH₂)ₙOH wherein n is selected from the group consisting of 3, 4, and 5, and

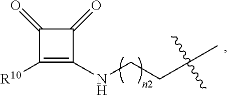

wherein

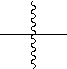

denotes a point of attachment, R¹⁰ is NH(C₁₋₆ alkyl), and n2 is selected from the group consisting of 1, 2, and 3.

In some embodiments of the compound of Formula (AII-g) or (AII-h), R⁴ is

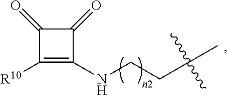

wherein R¹⁰ is NH(CH₃) and n2 is 2.

In some embodiments of the compound of Formula (AII-g) or (AII-h), R⁴ is —(CH₂)₂OH.

In some embodiments, the ionizable amino lipid may be one or more of compounds of Formula (VI):

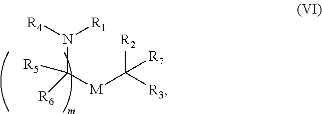

or their N-oxides, or salts or isomers thereof, wherein:
R₁ is selected from the group consisting of C₅₋₃₀ alkyl, C₅₋₂₀ alkenyl, —R*YR″, —YR″, and —R″M′R′;
R₂ and R₅ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle,
each X is independently selected from the group consisting of F, Cl, Br, and I; and
m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (VI) includes those in which:
R¹ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R⁷ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which:

R₁ is selected from the group consisting of C₅₋₃₀ alkyl, C₅₋₂₀ alkenyl, —R*YR″, —YR″, and —R″M′R′;
R₂ and R₅ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R⁴ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and -CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle,
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which

R; is selected from the group consisting of C₅₋₃₀ alkyl, C₅₋₂₀ alkenyl, —R*YR″, —YR″, and —R″M′R′;

R₂ and R₅ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R⁷ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In certain embodiments, a subset of compounds of Formula (VI) includes those of Formula (VI-A):

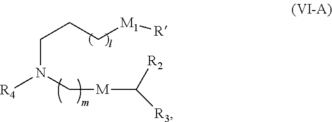

or its N-oxide, or a salt or isomer thereof, wherein l is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₅ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (VI) includes those of Formula (VI-B):

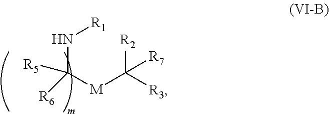

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is H, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (VI) includes those of Formula (VII):

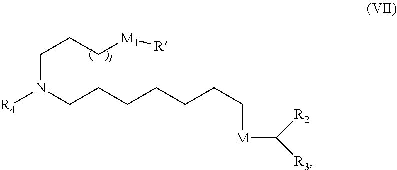

or its N-oxide, or a salt or isomer thereof, wherein l is selected from 1, 2, 3, 4, and 5; M₁ is a bond or M′; R⁴ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In one embodiment, the compounds of Formula (VI) are of Formula (VIIa),

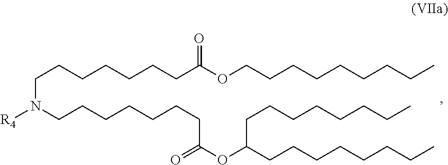

or their N-oxides, or salts or isomers thereof, wherein R₄ is as defined above.

In another embodiment, the compounds of Formula (VI) are of Formula (VIIb),

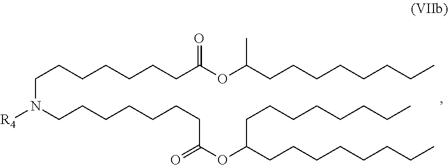

or their N-oxides, or salts or isomers thereof, wherein R₄ is as defined above.

In another embodiment, the compounds of Formula (VI) are of Formula (VIIc) or (VIIe):

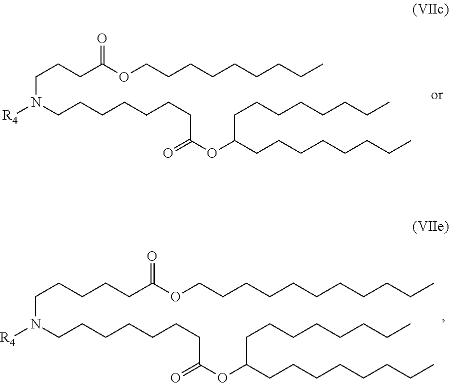

or their N-oxides, or salts or isomers thereof, wherein R₄ is as defined above.

In another embodiment, the compounds of Formula (VI) are of Formula (VIIf):

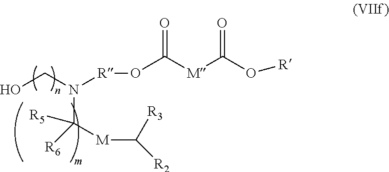

or their N-oxides, or salts or isomers thereof,
wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (VI) are of Formula (VIId),

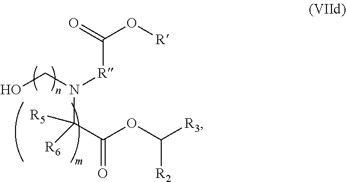

or their N-oxides, or salts or isomers thereof, wherein n is 2, 3, or 4; and m, R′, R″, and R₂ through R₆ are as defined above. For example, each of R₂ and R₃ may be independently selected from the group consisting of C₅₋₁₄ alkyl and C₅₋₁₄ alkenyl.

In some embodiments, an ionizable amino lipid comprises a compound having structure:

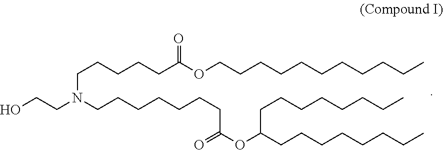 [SMILES: CCCCCCCCCCCOC(=O)CCCCCN(CCO)CCCCCCCC(=O)OC(CCCCCCCC)CCCCCCCC]

In some embodiments, an ionizable amino lipid comprises a compound having structure:

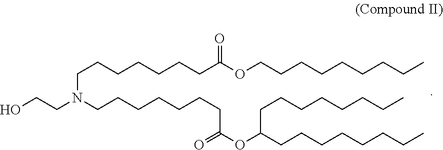 [SMILES: CCCCCCCCCOC(=O)CCCCCCCN(CCO)CCCCCCCC(=O)OC(CCCCCCCC)CCCCCCCC]

In a further embodiment, the compounds of Formula (VI) are of Formula (VIIg),

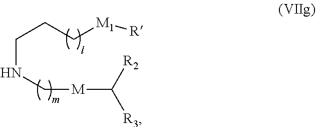

or their N-oxides, or salts or isomers thereof, wherein l is selected from 1, 2, 3, 4, and 5; m is selected from 5, 6, 7, 8, and 9; M₁ is a bond or M′; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

The central amine moiety of a lipid according to Formula (VI), (VI-A), (VI-B), (VII), (VIIa), (VIIb), (VIIc), (VIId), (VIIe), (VIIf), or (VIIg) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

In some embodiments, the ionizable amino lipid may be one or more of compounds of formula (VIII),

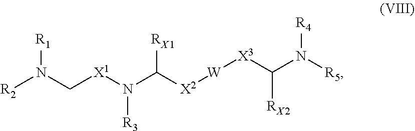

or salts or isomers thereof, wherein
W is

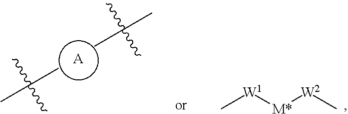

ring A is

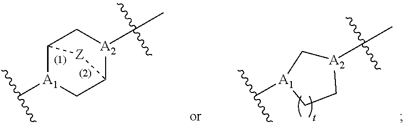

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O)—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

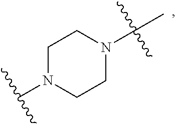 [SMILES: *N1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of any of formulae (VIIIa1)-(VIIIa8):

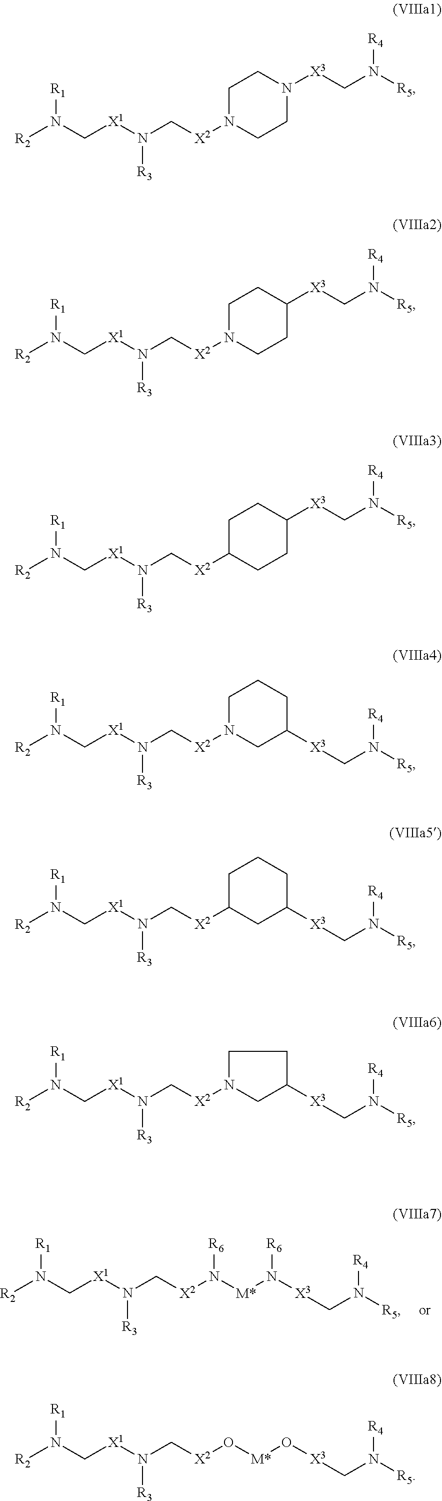

In some embodiments, the ionizable amino lipid is

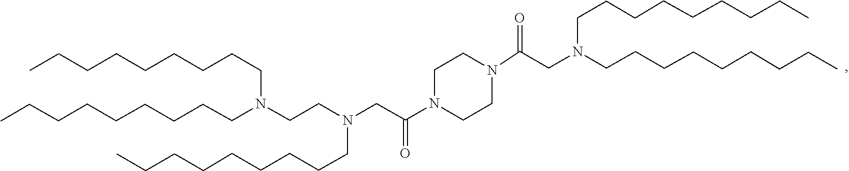 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (VIII), (VIIIa1), (VIIIa2), (VIIIa3), (VIIIa4), (VIIIa5), (VIIIa6), (VIIIa7), or (VIIIa8) may be protonated at a physiological pH. Thus, a lipid may have a positive or partial positive charge at physiological pH.

In some embodiments, the lipid nanoparticle comprises a lipid having the structure:

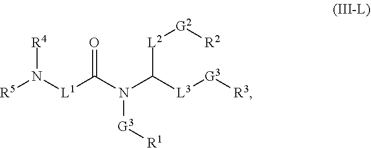

or a pharmaceutically acceptable salt, tautomer, or stereoisomer thereof, wherein:
R¹ is optionally substituted C₁-C₂₄ alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R⁴ and R⁵ join, along with the N to which they are attached, to form a heterocyclyl or heteroaryl;
L¹, L², and L³ are each independently optionally substituted C₁-C₁₈ alkylene;
G¹ is a direct bond, —(CH₂)ₙO(C═O)—, —(CH₂)ₙ(C═O)O—, or —(C═O)—;
G² and G³ are each independently —(C═O)O— or —O(C═O)—; and n is an integer greater than 0.

In some embodiments, the lipid nanoparticle comprises a lipid having the structure:

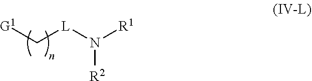

or a pharmaceutically acceptable salt, tautomer, or stereoisomer thereof, wherein:
G¹ is —N(R³)R⁴ or —OR⁵;
R¹ is optionally substituted branched, saturated or unsaturated C₁₂-C₃₆ alkyl;
R² is optionally substituted branched or unbranched, saturated or unsaturated C₁₂-C₃₆ alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
R⁵ is H or optionally substituted C₁-C₆ alkyl;
L is —C(═O)—, C₆-C₁₂ alkylene, C₆-C₁₂ alkenylene, or C₂-C₆ alkynylene; and
n is an integer from 1 to 12.

In some embodiments, the lipid nanoparticle comprises a lipid having the structure:

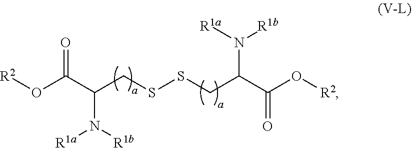

or a pharmaceutically acceptable salt thereof, wherein:
each R^{1a} is independently hydrogen, R^{1c}, or R^{1d};
each R^{1b} is independently R^{1c} or R^{1d};
each R^{1c} is independently —[CH₂]₂C(O)X¹R³;
each R^{1d} Is independently —C(O)R⁴;
each R² is independently —[C(R^{2a})₂]_{c}R^{2b};
each R^{2a} is independently hydrogen or C₁-C₆ alkyl;
R^{2b} is —N(L₁-B)₂; —(OCH₂CH₂)₆OH; or —(OCH₂CH₂)_{b}OCH₃;
each R³ and R⁴ is independently C₆-C₃₀ aliphatic;
each I₃ is independently C₁-C₁₀ alkylene;
each B is independently hydrogen or an ionizable nitrogen-containing group;
each X¹ is independently a covalent bond or O;
each a is independently an integer of 1-10;
each b is independently an integer of 1-10; and
each c is independently an integer of 1-10.

In some embodiments, the lipid nanoparticle comprises a lipid having the structure:

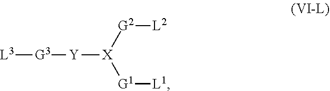

or a pharmaceutically acceptable salt, prodrug or stereoisomer thereof, wherein:
X is N, and Y is absent; or X is CR, and Y is NR;
L¹ is —O(C—O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;

L³ is —O(C═O)R³ or —(C═O)OR³;
G¹ and G² are each independently C₂-C₁₂ alkylene or C₂-C₁₂ alkenylene;
G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
each R is independently H or C₁-C₁₂ alkyl;
R¹, R² and R³ are each independently C₁-C₂₄ alkyl or C₂-C₂₄ alkenyl; and x is 0, 1 or 2, and
wherein each alkyl, alkenyl, alkylene, alkenylene, heteroalkylene and heteroalkenylene is independently substituted or unsubstituted unless otherwise specified.

In some embodiments, the lipid nanoparticle comprises a lipid having the structure:

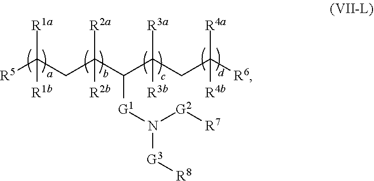

or a pharmaceutically acceptable salt, tautomer, prodrug or stereoisomer thereof, wherein:
L¹ and L² are each independently —O(C═O)—, —(C═O)O—, —C(═O)—, —O—, —S(O)x-ₛ; —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C—C₂ alkylene, —(C═O)—, —O(C═O)—, —SC(═O)—, —NR^{a}C(═O)— or a direct bond;
G² is —C(O)—, —(CO)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent RED and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4A} and R^{4B} are, at each occurrence, independently either: (a) H or C₁-C₁₂ alkyl; or (b) R^{4A} is H or C₁-C₁₂ alkyl, and R^{4B} together with the carbon atom to which it is bound is taken together with an adjacent R^{4B} and the carbon atom to which it is bound to form a carbon-carbon double bond;
R⁵ and R⁶ are each independently H or methyl;
R⁷ is H or C—C₂₀ alkyl;
R⁸ is OH, —N(R⁹)(C═O)R¹⁰, —(C═O)NR⁹R¹⁰, —NR⁹R¹⁰, —(C═O)OR″¹ or —O(C═O)R″, provided that G³ is C₄-C₆ alkylene when R⁸ is —NR⁹R¹⁰,
R⁹ and R¹⁰ are each independently H or C₁-C₁₂ alkyl;
R″ is aralkyl;
a, b, c and d are each independently an integer from 1 to 24; and x is 0, 1 or 2,
wherein each alkyl, alkylene and aralkyl is optionally substituted.

In some embodiments, the lipid nanoparticle comprises a lipid having the structure:

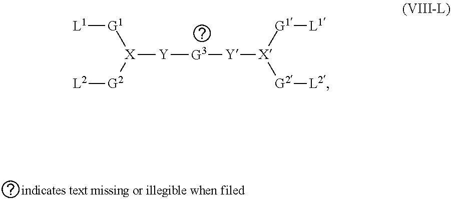

or a pharmaceutically acceptable salt, prodrug or stereoisomer thereof, wherein:
X and X′ are each independently N or CR;
Y and Y′ are each independently absent, —O(C═O)—, —(C═O)O— or NR, provided that:
   a) Y is absent when X is N;
   b) Y′ is absent when X′ is N;
   c) Y is —O(C═O)—, —(C═O)O— or NR when X is CR; and
   d) Y′ is —O(C═O)—, —(C═O)O— or NR when X′ is CR,
L¹ and L^{1′} are each independently —O(C═O)R′, —(C═O)OR′, —C(═O)R′, —OR¹, —S(O)_{z}R′, —S—SR¹, —C(═O)SR′, —SC(═O)R′, —NR^{a}C(═O)R′, —C(═O)NR^{b}R_{c}, —NR^{a}C(═O)NR^{b}R^{c}, —OC(═O)NR^{b}R^{c} or —NR^{a}C(═O)OR′;
L² and L^{2′} are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹, G^{1′}, G² and G^{2′} are each independently C₂-C₁₂ alkylene or C₂-C₁₂ alkenylene;
G is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂ alkenyl;
R^{c} and R^{f} are, at each occurrence, independently C₁-C₁₂ alkyl or C₂-C₁₂ alkenyl;
R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and wherein each alkyl, alkenyl, alkylene, alkenylene, heteroalkylene and heteroalkenylene is independently substituted or unsubstituted unless otherwise specified.

In some embodiments, the lipid nanoparticle comprises a lipid having the structure:

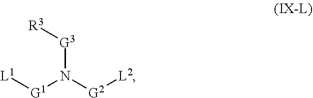

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R⁵;
R⁴ is C₁-C₁₂ alkyl;
R⁵ is substituted C₁-C₁₂ alkyl; and
x is 0, 1 or 2, and
wherein each alkyl, alkenyl, alkylene, alkenylene, cycloalkylene, cycloalkenylene, aryl and aralkyl is independently substituted or unsubstituted unless otherwise specified.

In some embodiments, the lipid nanoparticle comprises a lipid having the structure:

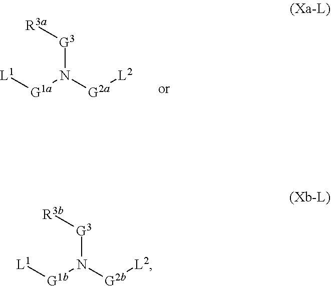

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R′, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G^{1a} and G^{2b} are each independently C₂-C₁₂ alkylene or C₂-C₁₂ alkenylene;
G^{1b} and G^{2b} are each independently C₁-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₂-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R^{3a} is —C(═O)N(R^{4a})R^{5a} or —C(═O)OR⁶;
R^{3b} is —NR^{4b}C(═O)R^{5b};
R^{4a} is C₁-C₁₂ alkyl;
R^{4b} is H, C₁-C₁₂ alkyl or C₂-C₁₂ alkenyl;
R^{5a} is H, C₁-C₈ alkyl or C₂-C₈ alkenyl;
R^{5b} is C₂-C₁₂ alkyl or C₂-C₁₂ alkenyl when R^{4b} is H; or R^{5b} is C₁-C₁₂ alkyl or C₂-C₁₂ alkenyl when R^{4b} is C₁-C₁₂ alkyl or C₂-C₁₂ alkenyl;
R⁶ is H, aryl or aralkyl; and
x is 0, 1 or 2, and
wherein each alkyl, alkenyl, alkylene, alkenylene, cycloalkylene, cycloalkenylene, aryl and aralkyl is independently substituted or unsubstituted.

In some embodiments, the lipid nanoparticle comprises a lipid having the structure:

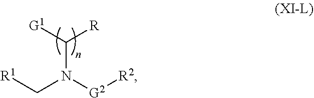

or a pharmaceutically acceptable salt, prodrug or stereoisomer thereof, wherein:
G¹ is —OH, —R³R⁴, —(C═O) R⁵ or —R³(C═O)R⁵;
G² is —CH₂— or —(C═O)—;
R is, at each occurrence, independently H or OH;
R¹ and R² are each independently optionally substituted branched, saturated or unsaturated C₁₂-C₃₆ alkyl;
R³ and R⁴ are each independently H or optionally substituted straight or branched, saturated or unsaturated C₁-C₆ alkyl;
R⁵ is optionally substituted straight or branched, saturated or unsaturated C₁-C₆ alkyl; and
n is an integer from 2 to 6.

In some embodiments, the lipid nanoparticle comprises a lipid having the structure:

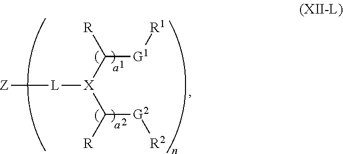

or a pharmaceutically acceptable salt, prodrug or stereoisomer thereof, wherein:
one of G¹ or G² is, at each occurrence, —O(C═O)—, —(C═O)O—, —C(═O)—, —O—, —S(O), —S—S—, —C(═O)S—, SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O—, and the other of G¹ or G² is, at each occurrence, —O(C═O)—, —(C═O)O—, —C(═O)—, —O—, —S(O), —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O— or a direct bond;
L is, at each occurrence, ˜O(C═O)—, wherein ˜ represents a covalent bond to X;
X is CR^{a};
Z is alkyl, cycloalkyl or a monovalent moiety comprising at least one polar functional group when n is 1; or Z is alkylene, cycloalkylene or a polyvalent moiety comprising at least one polar functional group when n is greater than 1;
R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;
R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
R¹ and R² have, at each occurrence, the following structure, respectively:

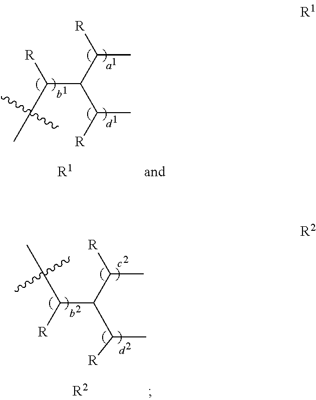

a¹ and a² are, at each occurrence, independently an integer from 3 to 12; b¹ and b² are, at each occurrence, independently 0 or 1;
c¹ and c² are, at each occurrence, independently an integer from 5 to 10; d¹ and d² are, at each occurrence, independently an integer from 5 to 10; y is, at each occurrence, independently an integer from 0 to 2; and n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

In some embodiments, the lipid nanoparticle comprises a lipid having the structure:

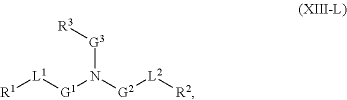

or a pharmaceutically acceptable salt, prodrug or stereoisomer thereof, wherein:
one of L¹ or L² is —O(C═O)—, —(C═O)O—, —C(═O)—, —O—, —S(O)—, —S—S—, —C(═O)S—, —SC(═O)—, —R^{a}C(═O)—, —C(═O)R^{a}—, R^{a}C(═O)R^{a}—, —OC(═O)R^{a}— or —R^{a}C(═O)O—, and the other of L¹ or L² is —O(C═O)—, —(C═O)O—, —C(═O)—, —O—, —S(O)ₓ—, —S—S—, —C(═O)S—, SC(═O)—, —R^{a}C(═O)—, —C(═O)R^{a}—, R^{a}C(═O)R^{a}—, —OC(═O) R^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —R⁵C(═O)R⁴;
R⁴ is C₁-C₁₂ alkyl;
R⁵ is H or C₁-C₆ alkyl; and
x is 0, 1 or 2.

In some embodiments, the lipid nanoparticle comprises a lipid having the structure:

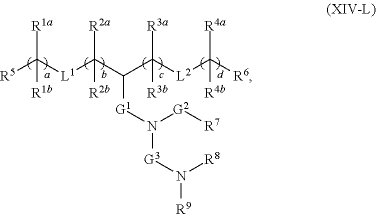

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, —R^{a}C(═O)R^{a}—, —OC(═O)R^{a}—, —R^{a}C(═O)O— or a direct bond;
G¹ is Ci-C₂ alkylene, —(C═O)—, —O(C═O)—, —SC(═O)—, —R^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and x is 0, 1 or 2.

In some embodiments, the lipid nanoparticle comprises a lipid having the structure:

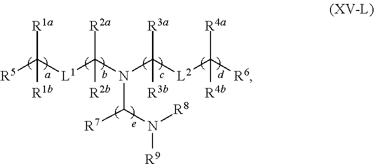

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom,
a and d are each independently an integer from 0 to 24; b and c are each independently an integer from 1 to 24; and e is 1 or 2,
provided that:
at least one of R^{1a}, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

In some embodiments, the lipid nanoparticle comprises a lipid having the structure:

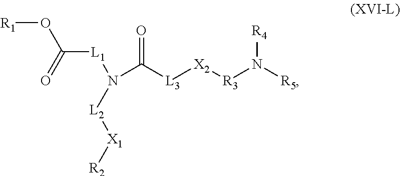

or a pharmaceutically acceptable salt thereof, wherein
R₁ and R₂ are the same or different, each a linear or branched alkyl with 1-9 carbons, or as alkenyl or alkynyl with 2 to 11 carbon atoms,
L₁ and L₂ are the same or different, each a linear alkyl having 5 to 18 carbon atoms, or form a heterocycle with N,
X₁ is a bond, or is —CG-G- whereby L2-CO—O—R₂ is formed,
X₂ is S or O,
L₃ is a bond or a lower alkyl, or form a heterocycle with N,
R₃ is a lower alkyl, and
R₄ and R₅ are the same or different, each a lower alkyl.

In some embodiments, the lipid nanoparticle comprises an ionizable lipid having the structure:

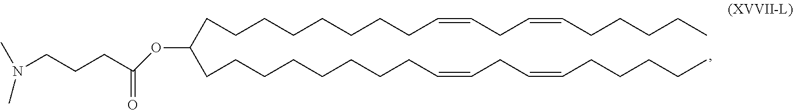 [SMILES: CCCCC/C=C\C/C=C\CCCCCCCCC(CCCCCCCC/C=C\C/C=C\CCCCC)OC(=O)CCCN(C)C]

or a pharmaceutically acceptable salt thereof.

In some embodiments, the lipid nanoparticle comprises a lipid having the structure:

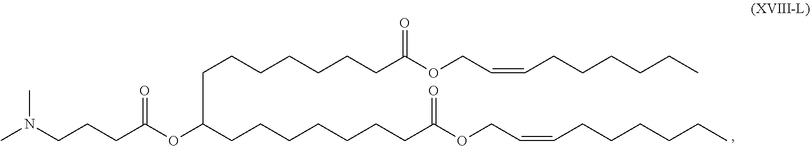 [SMILES: CCCCCC/C=C\COC(=O)CCCCCCCC(CCCCCCCC(=O)OC/C=C\CCCCCC)OC(=O)CCCN(C)C]

or a pharmaceutically acceptable salt thereof.

In some embodiments, the lipid nanoparticle comprises a lipid having the structure:

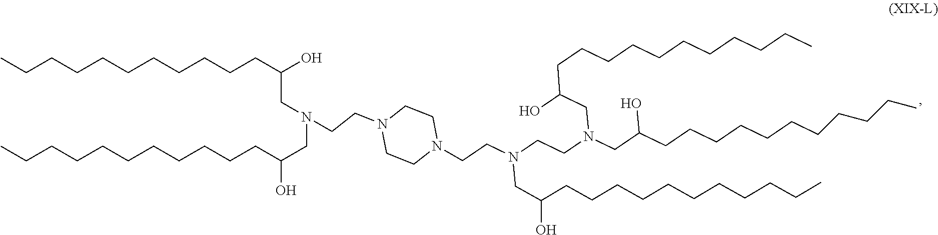 [SMILES: CCCCCCCCCCCC(O)CN(CCN1CCN(CCN(CC(O)CCCCCCCCCCC)CC(O)CCCCCCCCCCC)CC1)CCN(CC(O)CCCCCCCCCCC)CC(O)CCCCCCCCCCC]

or a pharmaceutically acceptable salt thereof.

In some embodiments, the lipid nanoparticle comprises a lipid having the structure:

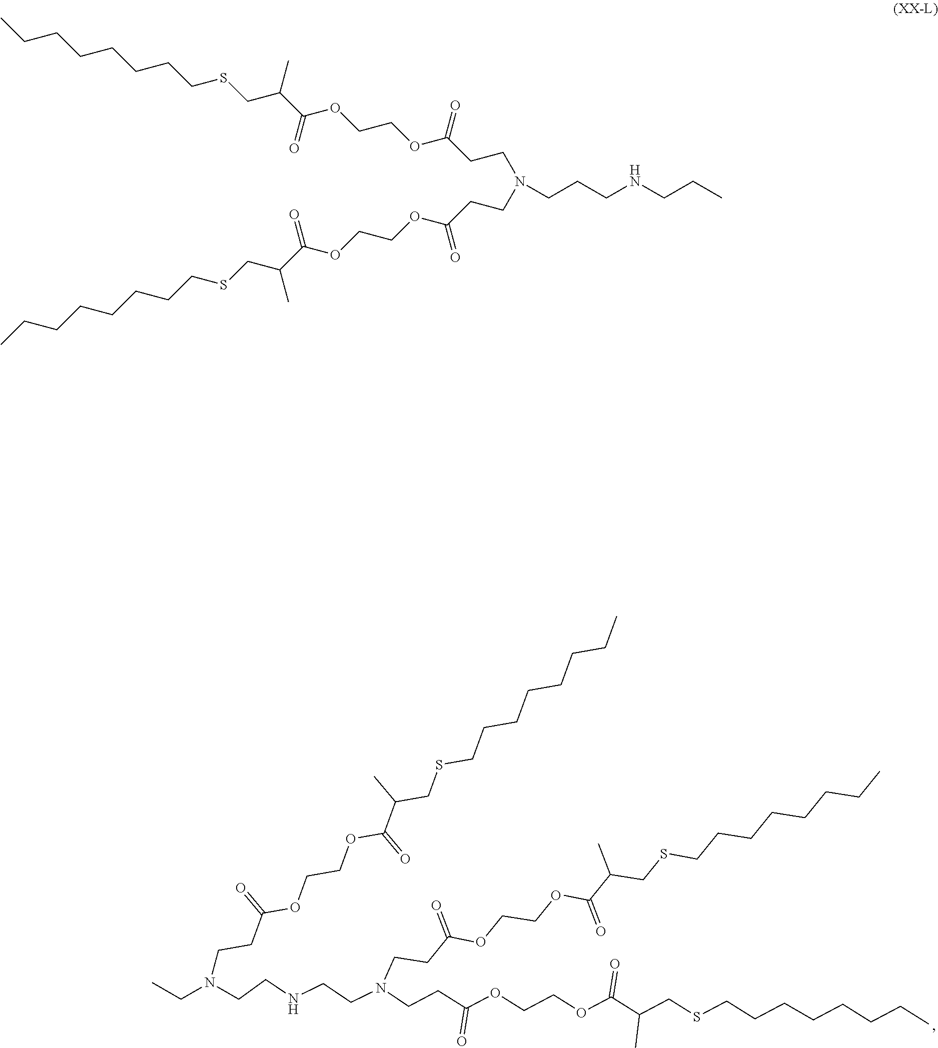

or a pharmaceutically acceptable salt thereof.

In some embodiments, the lipid nanoparticle comprises a lipid having the structure:

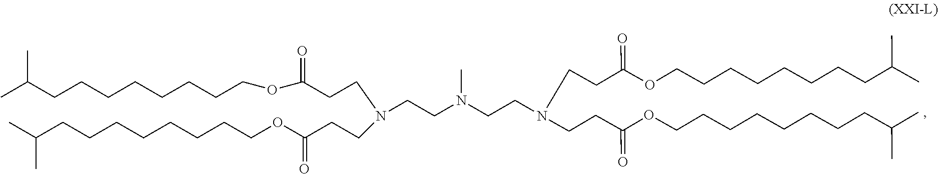 [SMILES: CC(C)CCCCCCCCOC(=O)CCN(CCC(=O)OCCCCCCCCC(C)C)CCN(C)CCN(CCC(=O)OCCCCCCCCC(C)C)CCC(=O)OCCCCCCCCC(C)C]

or a pharmaceutically acceptable salt thereof.

In some embodiments, the lipid nanoparticle comprises a lipid having the structure:

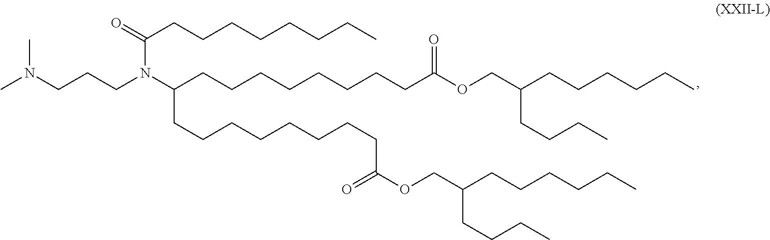 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

or a pharmaceutically acceptable salt thereof.

In some embodiments, the lipid nanoparticle comprises a lipid having the structure:

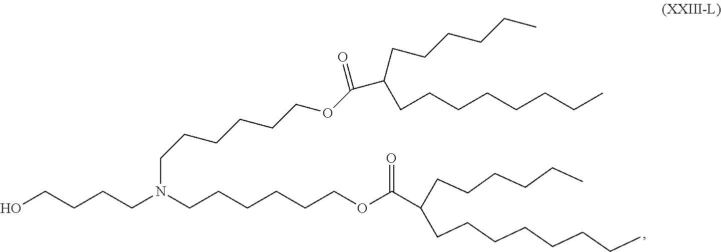 [SMILES: CCCCCCCCC(CCCCCC)C(=O)OCCCCCCN(CCCCO)CCCCCCOC(=O)C(CCCCCC)CCCCCCCC]

or a pharmaceutically acceptable salt thereof.

In some embodiments, the lipid nanoparticle comprises a lipid having the structure:

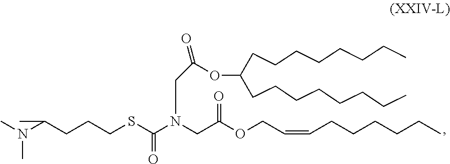 [SMILES: CCCCCC/C=C\COC(=O)CN(CC(=O)OC(CCCCCCCC)CCCCCCCC)C(=O)SCCCC(C)N(C)C]

or a pharmaceutically acceptable salt thereof.

In some embodiments, the lipid nanoparticle comprises a lipid having the structure:

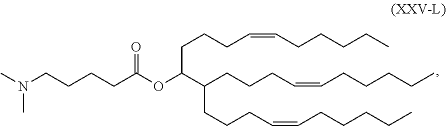 [SMILES: CCCCC/C=C\CCCC(CCC/C=C\CCCCC)C(CCC/C=C\CCCCC)OC(=O)CCCCN(C)C]

or a pharmaceutically acceptable salt thereof.

In some embodiments, the lipid nanoparticle comprises a lipid having the structure:

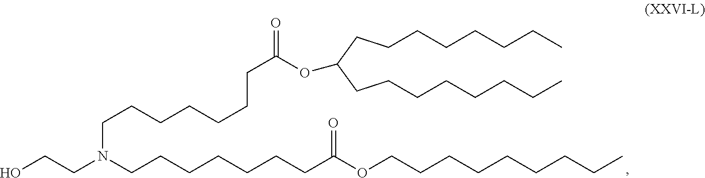 [SMILES: CCCCCCCCCOC(=O)CCCCCCCN(CCO)CCCCCCCC(=O)OC(CCCCCCCC)CCCCCCCC]

or a pharmaceutically acceptable salt thereof.

In some embodiments, the lipid nanoparticle comprises a lipid having the structure:

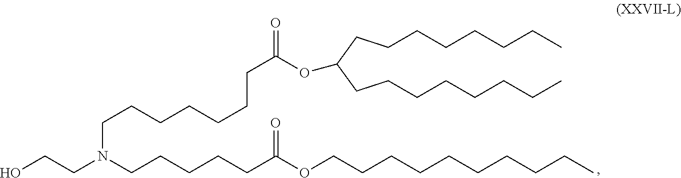 [SMILES: CCCCCCCCCCOC(=O)CCCCCN(CCO)CCCCCCCC(=O)OC(CCCCCCCC)CCCCCCCC]

or a pharmaceutically acceptable salt thereof.

### Non-Cationic Lipids

In certain embodiments, the lipid nanoparticles provided herein comprise one or more non-cationic lipids. Non-cationic lipids may be phospholipids.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, a non-cationic lipid comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In some embodiments, the lipid nanoparticle comprises 5-15 mol %, 5-10 mol %, or 10-15 mol % DSPC. For example, the lipid nanoparticle may comprise 5, 6, 7, 8, 9, 10, 11, 12, 13, 14, or 15 mol % DSPC.

In certain embodiments, the lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, crucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidyl glycerols, and phosphatidic acids. Phospholipids also include phosphosphingolipid, such as sphingomyelin.

In some embodiments, a phospholipid is selected from 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C₁₋₆ Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid is an analog or variant of DSPC. In certain embodiments, a phospholipid is a compound of Formula (IX).

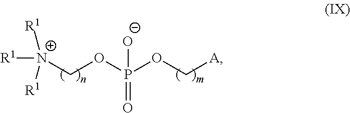

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

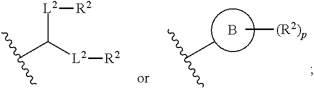

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O), S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

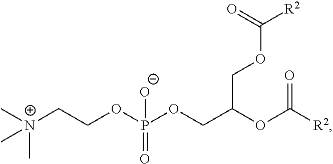

wherein each instance of R² is independently unsubstituted alkyl, unsubstituted alkenyl, or unsubstituted alkynyl.

In some embodiments, the phospholipids may be one or more of the phospholipids described in PCT Application No. PCT/US2018/037922.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises a molar ratio of 20-60% amino lipid, 5-25% phospholipid, 25-55% structural lipid, and 0.5-15% PEG lipid.

In some embodiments, the lipid nanoparticle comprises a molar ratio of 20-60% amino lipid, 5-30% phospholipid, 10-55% structural lipid, and 0.5-15% PEG lipid.

### Structural Lipids

The lipid composition of a pharmaceutical composition disclosed herein can comprise one or more structural lipids. As used herein, the term “structural lipid” includes sterols and also lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As used herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

In some embodiments, the lipid nanoparticle comprises 30-45 mol % sterol, optionally 35-40 mol %, for example, 30-31 mol %, 31-32 mol %, 32-33 mol %, 33-34 mol %, 35-35 mol %, 35-36 mol %, 36-37 mol %, 38-38 mol %, 38-39 mol %, or 39-40 mol %. In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 35-40 mol % cholesterol. For example, the lipid nanoparticle may comprise 35, 35.5, 36, 36.5, 37, 37.5, 38, 38.5, 39, 39.5, or 40 mol % cholesterol.

### Polyethylene Glycol (PEG)-Lipids

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-lipid). However, it has been discovered that certain components may decrease the stability of encapsulated nucleic acids (e.g., mRNA molecules). The reduced stability may limit the broad applicability of the particulate carriers. As such, there remains a need for methods by which to improve the stability of nucleic acid (e.g., mRNA) encapsulated within lipid nanoparticles.

The lipid composition of a pharmaceutical composition disclosed herein can comprise one or more polyethylene glycol (PEG) lipids. As used herein, the term “PEG-lipid” or “PEG-modified lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but is not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-distearyl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In some embodiments, the PEG-lipid is PEG₂ₖ-DMG.

In some embodiments, the lipid nanoparticles provided herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae provided herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

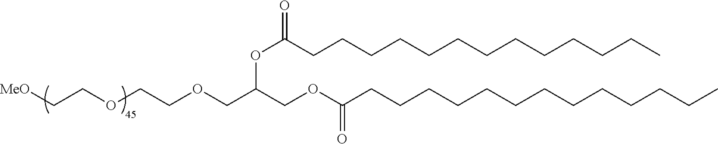

In some embodiments, PEG lipids can be PEGylated lipids described in International Publication No WO2012099755, the contents of which is herein incorporated by reference in its entirety. Any exemplary PEG lipids may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment.

In certain embodiments, a PEG lipid is a compound of Formula (X):

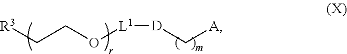

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S. C(O), C(O)N(R^{N}), NR^{N}C(O), C(O)O, OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

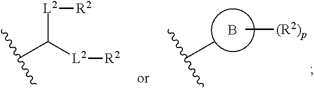

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S. C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (X) is a PEG-OH lipid (i.e., R³ is —OR^{O}, and R^{O} is hydrogen). In certain embodiments, the compound of Formula (X) is of Formula (X-OH):

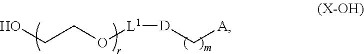

or a salt thereof.

In certain embodiments, a PEG lipid is a PEGylated fatty acid. In certain embodiments, a PEG lipid is a compound of Formula (XI). Provided herein are compounds of Formula (XI):

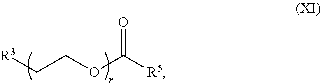

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), —C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), —C(S)N(R^{N}), NR^{N}C(S), NR^{N}C(S)N(R^{N}), S(O), OS(O), S(O)O, OS(O)O, OS(O)₂, S(O)₂O, —OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (XI) is of Formula (XI-OH):

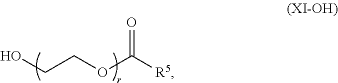

or a salt thereof. In some embodiments, r is 40-50.

In yet other embodiments the compound of Formula (XI) is:

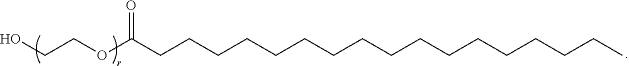

or a salt thereof.

In one embodiment, the compound of Formula (XI) is

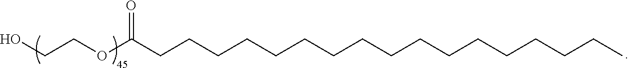

In some embodiments, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, the lipid nanoparticle comprises 1-5% PEG-modified lipid, optionally 1-3 mol %, for example 1.5 to 2.5 mol %, 1-2 mol %, 2-3 mol %, 3-4 mol %, or 4-5 mol %. In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

In some embodiments, the lipid nanoparticle comprises 20-60 mol % ionizable amino lipid, 5-25 mol % non-cationic lipid, 25-55 mol % sterol, and 0.5-15 mol % PEG-modified lipid.

In some embodiments, a LNP comprises an ionizable amino lipid of Compound I, wherein the non-cationic lipid is DSPC, the structural lipid that is cholesterol, and the PEG lipid is DMG-PEG.

In some embodiments, a LNP comprises an ionizable amino lipid of any of Formula VI, VII or VIIII, a phospholipid comprising DSPC, a structural lipid, and a PEG lipid comprising PEG-DMG.

In some embodiments, a LNP comprises an ionizable amino lipid of any of Formula VI, VII or VIII, a phospholipid comprising DSPC, a structural lipid, and a PEG lipid comprising a compound having Formula XI.

In some embodiments, a LNP comprises an ionizable amino lipid of Formula VI, VII or VIII, a phospholipid comprising a compound having Formula VIII, a structural lipid, and the PEG lipid comprising a compound having Formula X or XI.

In some embodiments, a LNP comprises an ionizable amino lipid of Formula VI, VII or VIII, a phospholipid comprising a compound having Formula IX, a structural lipid, and the PEG lipid comprising a compound having Formula X or XI.

In some embodiments, a LNP comprises an ionizable amino lipid of Formula VI, VII or VIII, a phospholipid having Formula IX, a structural lipid, and a PEG lipid comprising a compound having Formula XI.

In some embodiments, the lipid nanoparticle comprises 49 mol % ionizable amino lipid, 10 mol % DSPC, 38.5 mol % cholesterol, and 2.5 mol % DMG-PEG.

In some embodiments, the lipid nanoparticle comprises 49 mol % ionizable amino lipid, 11 mol % DSPC, 38.5 mol % cholesterol, and 1.5 mol % DMG-PEG.

In some embodiments, the lipid nanoparticle comprises 48 mol % ionizable amino lipid, 11 mol % DSPC, 38.5 mol % cholesterol, and 2.5 mol % DMG-PEG.

In some embodiments, the lipid nanoparticles comprise one or more of ionizable molecules, polynucleotides, and optional components, such as structural lipids, sterols, neutral lipids, phospholipids and a molecule capable of reducing particle aggregation (e.g., polyethylene glycol (PEG), PEG-modified lipid), such as those described above.

Some aspects provide lipid compositions comprising a lipid and a compound of compound of Formula I or of Formula II, or a tautomer or solvate thereof.

A lipid composition may comprise one or more lipids. Such lipids may include those useful in the preparation of lipid nanoparticle formulations as provided herein or as known in the art.

The compositions provided herein are also useful for treating or preventing a symptom of diseases characterized by missing or aberrant protein activity, by replacing the missing protein activity or overcoming the aberrant protein activity.

In some embodiments, a subject to which a composition comprising a nucleic acid, a lipid, and/or a compound of Formula I or Formula II is administered is a subject that suffers from or is at risk of suffering from a disease, disorder or condition, including a communicable or non-communicable disease, disorder or condition. As used herein, “treating” a subject can include either therapeutic use or prophylactic use relating to a disease, disorder or condition, and may be used to describe uses for the alleviation of symptoms of a disease, disorder or condition, uses for vaccination against a disease, disorder or condition, and uses for decreasing the contagiousness of a disease, disorder or condition, among other uses.

In some embodiments the nucleic acid is an mRNA vaccine designed to achieve particular biologic effects. Exemplary vaccines feature mRNAs encoding a particular antigen of interest (or an mRNA or mRNAs encoding antigens of interest). In exemplary aspects, the vaccines feature an mRNA or mRNAs encoding antigen(s) derived from infectious diseases or cancers.

In some embodiments, microbial growth within a composition disclosed herein is inhibited. In some embodiments, microbial growth is inhibited by the compound (e.g., compound of Formula I or Formula II). In some embodiments, a composition disclosed herein does not comprise a pharmaceutical preservative. Non-limiting examples of pharmaceutical preservatives include methyl paragen, ethyl paraben, propyl paraben, butyl paraben, benzyl alcohol, chlorobutanol, phenol, meta cresol (m-cresol), chloro cresol, benzoic acid, sorbic acid, thiomersal, phenylmercuric nitrate, bronopol, propylene glycol, benzalkonium chloride, and benzethonium chloride. In some embodiments, a composition disclosed herein does not comprise phenol, m-cresol, or benzyl alcohol. Compositions in which microbial growth is inhibited may be useful in the preparation of injectable formulations, including those intended for dispensing from multi-dose vials. Multi-dose vials refer to containers of pharmaceutical compositions from which multiple doses can be taken repeatedly from the same container. Compositions intended for dispensing from multi-dose vials typically must meet USP requirements for antimicrobial effectiveness. In some embodiments, a composition disclosed herein comprising a compound (e.g., a compound of Formula I, or of Formula II) has antimicrobial effectiveness, and may be dispensed from a multi-dose vial.

In some embodiments, “administering” or “administration” means providing a material to a subject in a manner that is pharmacologically useful. In some embodiments, a composition disclosed herein is administered to a subject enterally. In some embodiments, an enteral administration of the composition is oral. In some embodiments, a composition disclosed herein is administered to the subject parenterally. In some embodiments, a composition disclosed herein is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs.

To “treat” a disease as the term is used herein, means to reduce the frequency or severity of at least one sign or symptom of a disease, disorder or condition experienced by a subject. The compositions provided herein are typically administered to a subject in an effective amount, that is, an amount capable of producing a desirable result. The desirable result will depend upon the active agent being administered. For example, an effective amount of a composition comprising a nucleic acid, a lipid, and a compound of Formula I may be an amount of the composition that is capable of increasing expression of a protein in the subject. A therapeutically acceptable amount may be an amount that is capable of treating a disease or condition, e.g., a disease or condition that that can be relieved by increasing expression of a protein in a subject. As is well known in the medical and veterinary arts, dosage for any one subject depends on many factors, including the subject's size, body surface area, age, the particular composition to be administered, the active ingredient(s) in the composition, the intended outcome of the administration, time and route of administration, general health, and other drugs being administered concurrently.

In some embodiments, a subject is administered a composition comprising a nucleic acid, a lipid, and/or a compound of Formula I in an amount sufficient to increase expression of a protein in the subject.

### Methods of Formulating

Also provided are methods of formulating nucleic acids. Some embodiments comprise adding a stabilizer compound to a composition comprising a nucleic acid and a lipid. In some embodiments, a method of formulating a nucleic acid comprises adding to a composition comprising a nucleic acid and a lipid, a compound of Formula I, or a tautomer or solvate thereof, to obtain a formulated composition. In certain embodiments, the compound of Formula I is of Formula Ia, Formula Ib, or Formula Ic, or a tautomer or solvate thereof.

In some embodiments, a method of formulating a nucleic acid comprises adding to a composition comprising a nucleic acid and a lipid, a compound of Formula II, or a tautomer or solvate thereof, to obtain a formulated composition. In certain embodiments, the compound of Formula II is of Formula IIa, or a tautomer or solvate thereof.

In some embodiments, the stabilizer compound is added to a composition comprising mRNA, and then the mRNA/stabilizer composition is used in the formation of a LNP. In some embodiments, the mRNA, stabilizer compound, and one or more LNP components are independently mixed together to form a LNP composition comprising mRNA and the stabilizer compound. In some embodiments, the stabilizer compound is added to a mRNA-encapsulated LNP. In some embodiments, the stabilizer compound is added to the LNP in a composition that comprises one or more components of the LNP, such as an ionizable lipid, a non-cationic lipid, and sterol, and/or a PEG-lipid.

In some embodiments, the stabilizer compound is added to a composition (e.g., containing mRNA, mRNA-encapsulated LNPS, or LNP components) at a pH of about 3.5 to about 8.5, such as from about 4 to about 8, about 4.5 to about 7.5, about 4, about 4.5, about 5, about 5.5, about 6, about 6.5, about 7, about 7.4, about 7.5, about 7.6, or about 8.

In some embodiments, the compositions containing the stabilizer compound are prepared and packaged under conditions that minimize, inhibit, or prevent exposure of the composition to light (e.g., room light, sunlight, UV light, and/or fluorescent light). In some embodiments, the compositions are prepared and/or packaged in the absence of one or more of room light, sunlight, UV light, and fluorescent light. In some embodiments, the compositions are prepared and/or packaged in the presence of red light, i.e., light having a frequency or frequencies in the range of 630-740 nm. In some embodiments, compositions containing the stabilizer compound are exposed to light (e.g., room light, UV light, and/or fluorescent light) for a period of 24 hours or less, such as 20 hours or less, 15 hours or less, 10 hours or less, 5 hours or less, 3 hours or less, 2 hours or less, 1 hour or less, 45 minutes or less, 30 minutes or less, 15 minutes or less, 10 minutes or less, or 5 minutes or less.

In certain embodiments, LNP preparations (e.g., populations or formulations) are analyzed for polydispersity in size (e.g., particle diameter) and/or composition (e.g., amino lipid amount or concentration, phospholipid amount or concentration, structural lipid amount or concentration, PEG-lipid amount or concentration, mRNA amount (e.g., mass) or concentration) and, optionally, further assayed for in vitro and/or in vivo activity. Fractions or pools thereof can also be analyzed for accessible mRNA and/or purity (e.g., purity as determined by reverse-phase (RP) chromatography).

Particle size (e.g., particle diameter) can be determined by Dynamic Light Scattering (DLS). DLS measures a hydrodynamic diameter. Smaller particles diffuse more quickly, leading to faster fluctuations in the scattering intensity and shorter decay times for the autocorrelation function. Larger particles diffuse more slowly, leading to slower fluctuations in the scattering intensity and longer decay times in the autocorrelation function.

mRNA purity can be determined by reverse phase high-performance liquid chromatography (RP-HPLC) size based separation. This method can be used to assess mRNA integrity by a length-based gradient RP separation and UV detection of RNA at 260 nm. As used herein “main peak” or “main peak purity” refers to the RP-HPLC signal detected from mRNA that corresponds to the full size mRNA molecule loaded within a given LNP formulation. mRNA purity can also be assessed by fragmentation analysis. Fragmentation analysis (FA) is a method by which nucleic acid (e.g., mRNA) fragments can be analyzed by capillary electrophoresis. Fragmentation analysis involves sizing and quantifying nucleic acids (e.g., mRNA), for example by using an intercalating dye coupled with an LED light source. Such analysis may be completed, for example, with a Fragment Analyzer from Advanced Analytical Technologies, Inc.

Compositions formed via the methods provided herein may be particularly useful for administering an agent to a subject in need thereof. In some embodiments, the compositions are used to deliver a pharmaceutically active agent. In some instances, the compositions are used to deliver a prophylactic agent. The compositions may be administered in any way known in the art of drug delivery, for example, orally, parenterally, intravenously, intramuscularly, subcutaneously, intradermally, transdermally, intrathecally, submucosally, sublingually, rectally, vaginally, etc.

Once the compositions have been prepared, they may be combined with pharmaceutically acceptable excipients to form a pharmaceutical composition. As would be appreciated by one of skill in this art, the excipients may be chosen based on the route of administration as described below, the agent being delivered, and the time course of delivery of the agent.

Pharmaceutical compositions provided herein and for use in accordance with the embodiments provided herein may include a pharmaceutically acceptable excipient. As used herein, the term “pharmaceutically acceptable excipient” means a non-toxic, inert solid, semi-solid or liquid filler, diluent, encapsulating material or formulation auxiliary of any type. Some examples of materials which can serve as pharmaceutically acceptable excipients are sugars such as lactose, glucose, and sucrose, starches such as corn starch and potato starch; cellulose and its derivatives such as sodium carboxymethyl cellulose, methylcellulose, hydroxypropylmethylcellulose, ethyl cellulose, and cellulose acetate; powdered tragacanth; malt; gelatin; talc; excipients such as cocoa butter and suppository waxes; oils such as peanut oil, cottonseed oil, safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen free water; isotonic saline; citric acid, acetate salts, Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions can be administered to humans and/or to animals, orally, rectally, parenterally, intracisternally, intravaginally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., the particles), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, ethanol, U.S.P., and isotonic sodium chloride solution. In addition, sterile, fixed oils are conventionally employed as a solvent or suspending medium. For this purpose any bland fixed oil can be employed including synthetic mono or diglycerides. In addition, fatty acids such as oleic acid are used in the preparation of injectables.

The injectable formulations can be sterilized, for example, by filtration through a bacteria retaining filter, or by incorporating sterilizing agents in the form of sterile solid compositions which can be dissolved or dispersed in sterile water or other sterile injectable medium prior to use.

Compositions for rectal or vaginal administration may be suppositories which can be prepared by mixing the particles with suitable non irritating excipients or carriers such as cocoa butter, polyethylene glycol, or a suppository wax which are solid at ambient temperature but liquid at body temperature and therefore melt in the rectum or vaginal cavity and release the particles.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard filled gelatin capsules using such excipients as lactose or milk sugar as well as high molecular weight polyethylene glycols and the like.

The solid dosage forms of tablets, dragées, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also possible.

The ointments, pastes, creams, and gels may contain, in addition to the compositions provided herein, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the compositions provided herein, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compositions in a proper medium. Absorption enhancers can also be used to increase the flux of the compound across the skin. The rate can be controlled by either providing a rate controlling membrane or by dispersing the compositions in a polymer matrix or gel.

In other embodiments, the stabilized compositions are loaded and stored in prefilled syringes and cartridges for patient-friendly autoinjector and infusion pump devices.

Kits for use in preparing or administering the compositions are also provided. A kit for forming compositions may include any solvents, solutions, buffer agents, acids, bases, salts, targeting agent, etc. needed in the composition formation process. Different kits may be available for different targeting agents. In certain embodiments, the kit includes materials or reagents for purifying, sizing, and/or characterizing the resulting compositions. The kit may also include instructions on how to use the materials in the kit. The one or more agents (e.g., pharmaceutically active agent) to be contained within the composition are typically provided by the user of the kit.

Kits are also provided for using or administering the compositions. The compositions may be provided in convenient dosage units for administration to a subject. The kit may include multiple dosage units. For example, the kit may include 1-100 dosage units. In certain embodiments, the kit includes a week supply of dosage units, or a month supply of dosage units. In certain embodiments, the kit includes an even longer supply of dosage units. The kits may also include devices for administering the compositions. Exemplary devices include syringes, spoons, measuring devices, etc. The kit may optionally include instructions for administering the compositions (e.g., prescribing information).

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds provided herein include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The terms “composition” and “formulation” are used interchangeably.

As used herein, the term “intercalating small molecule” or “small molecule nucleic acid intercalating agent” refers to a compound containing aromatic or heteroaromatic ring systems that can insert between adjacent base pairs of double stranded DNA or folded or double stranded regions of mRNA. Intercalating agents typically but not necessarily, contain planar polyaromatic rings and cationic substituents. Intercalation between adjacent base pairs may be full or partial. A typical small molecule intercalating agent contains three or four fused rings that absorb light in the UV-visible region of the electromagnetic spectrum.

The following examples are intended to illustrate certain non-limiting embodiments. Without further elaboration, it is believed that one skilled in the art can, based on the above description, utilize the disclosed compositions and methods to the fullest extent. The following specific embodiments are, therefore, to be construed as merely illustrative, and not limitative in any way whatsoever.

### Embodiments

The following embodiments are provided.

Embodiment A. A stabilized pharmaceutical composition comprising a nucleic acid, a lipid, and a stabilizing compound that reduces adduct formation and/or nucleic acid degradation in the composition.

Embodiment B. A stabilized pharmaceutical composition comprising a nucleic acid, a lipid, and a stabilizing compound that physically interacts with the nucleic acid.

Embodiment C. A lipid nanoparticle comprising a nucleic acid and a stabilizing compound that physically interacts with the nucleic acid.

Embodiment D. A stabilized pharmaceutical composition comprising a nucleic acid, a lipid, and an intercalating stabilizer that binds to the nucleic acid with a micromolar dissociation constant.

Embodiment E. A stabilized pharmaceutical composition comprising a lipid nanoparticle comprising mRNA and a stabilizing compound reversibly bound to double stranded regions of the mRNA.

Embodiment F. A stabilized pharmaceutical composition comprising a nucleic acid and an intercalating stabilizer, wherein the composition does not contain an excipient stabilizer that functions via a chemical reactivity mechanism.

Embodiment G. A stabilized nucleic acid comprising a mRNA reversibly bound to an intercalating small molecule that is cationic, soluble in aqueous solution, and capable of permeating a lipid nanoparticle.

Embodiment H. A stabilized pharmaceutical composition comprising a nucleic acid bound to a stabilizing compound, wherein the composition is substantially free of unbound stabilizing compound.

Embodiment I. A stabilized pharmaceutical composition comprising lipid nanoparticles comprising a nucleic acid and a stabilizing compound, wherein substantially all of the stabilizing compound is located in or on the lipid nanoparticles.

Embodiment J. A stabilized pharmaceutical composition comprising a lipid nanoparticle comprising mRNA and an amount of an isoquinoline alkaloid, or a derivative thereof, effective to stabilize the composition.

Embodiment K. A stabilized pharmaceutical composition comprising a nucleic acid, a lipid, and a stabilizing compound that reduces adduct formation and/or nucleic acid degradation in the composition, wherein the stabilizing compound is not a compound of the formula:

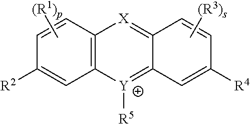

or an acceptable salt, tautomer, reduced form, or oxidized form thereof, wherein:
Y is N, S, or O;
X is N—R⁵, S, O, or C—R^{C};
R² and R⁴ are each independently —N(R^{N})₂,
each R⁵ is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or is absent;
each instance of R¹ and R³ is independently halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, optionally substituted sulfonyl, —OR^{O}, —N(R^{N})₂, or —SR^{S},
p is 0, 1, 2, or 3;
s is 0, 1, 2, or 3;
each instance of R^{O} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or an oxygen protecting group;
each instance of R^{N} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; or two R^{N} bonded to the name nitrogen atom are taken together with the intervening atoms to form optionally substituted heterocyclyl or optionally substituted heteroaryl;
each instance of R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; and
R^{C} is hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, optionally substituted sulfinyl, optionally substituted sulfonyl, —OR^{O}, —N(R^{N})₂, or —SR^{S}.

Embodiment 1. A stabilized pharmaceutical composition comprising a lipid nanoparticle (LNP) encapsulated nucleic acid and a stabilizing compound that reduces adduct formation between the nucleic acid and a lipid of the LNP and/or nucleic acid degradation in the composition.

Embodiment 2. A stabilized pharmaceutical composition comprising a lipid nanoparticle (LNP) encapsulated nucleic acid and a stabilizing compound that physically interacts with the nucleic acid.

Embodiment 3. A lipid nanoparticle (LNP) comprising a nucleic acid and a stabilizing compound that physically interacts with the nucleic acid.

Embodiment 4. A stabilized pharmaceutical composition comprising a lipid nanoparticle (LNP) encapsulated nucleic acid and an intercalating stabilizer that binds to the nucleic acid with a micromolar dissociation constant.

Embodiment 5. A stabilized pharmaceutical composition comprising a lipid nanoparticle (LNP) comprising mRNA and a stabilizing compound reversibly bound to double stranded regions of the mRNA.

Embodiment 6. A stabilized pharmaceutical composition comprising a lipid nanoparticle (LNP) encapsulated nucleic acid and an intercalating stabilizer, wherein the composition does not contain an excipient stabilizer that functions via a chemical reactivity mechanism.

Embodiment 7. A stabilized pharmaceutical composition comprising a lipid nanoparticle (LNP) encapsulated mRNA reversibly bound to an intercalating small molecule that is cationic, soluble in aqueous solution, and capable of permeating a lipid nanoparticle.

Embodiment 8. A stabilized pharmaceutical composition comprising lipid nanoparticle (LNP) encapsulated nucleic acid bound to a stabilizing compound, wherein the composition is substantially free of unbound stabilizing compound.

Embodiment 9. A stabilized pharmaceutical composition comprising lipid nanoparticles comprising a nucleic acid and a stabilizing compound, wherein substantially all of the stabilizing compound is located in or on the lipid nanoparticles.

Embodiment 10. A stabilized pharmaceutical composition comprising a lipid nanoparticle (LNP) encapsulated mRNA and an amount of an isoquinoline alkaloid, or a derivative thereof, effective to stabilize the composition.

Embodiment 11. The stabilized pharmaceutical composition of any of the preceding Embodiments, wherein the LNP comprises a molar ratio of about 20-60% ionizable cationic lipid:about 5-25% non-cationic lipid:about 25-55% sterol; and about 0.5-15% PEG-modified lipid.

Embodiment 12. A stabilized pharmaceutical composition comprising: a nucleic acid formulation comprising a nucleic acid and a lipid, and a compound of Formula I:

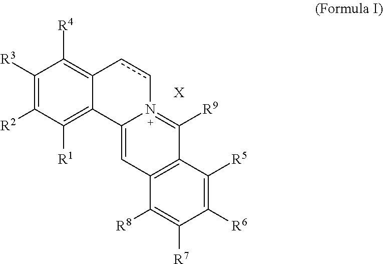

or a tautomer or solvate thereof, wherein:
[symbol] is a single bond or a double bond;
R¹ is H;
R² is OCH₃, or together with R³ is OCH₂O;
R³ is OCH₃, or together with R² is OCH₂O;
R⁴ is H;
R⁵ is H or OCH₃;
R⁶ is OCH₃;
R⁷ is H or OCH₃;
R⁸ is H;
R⁹ is H or CH₃; and
X is a pharmaceutically acceptable anion;
or a compound of Formula II:

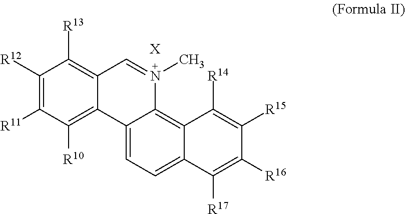

or a tautomer or solvate thereof, wherein:

R¹⁰ is H;

R¹¹ is H;
R¹² together with R¹³ is OCH₂O;
R¹⁴ is H;
R¹⁵ together with R¹⁶ is OCH₂O;
R¹⁷ is H; and
X is a pharmaceutically acceptable anion.

Embodiment 13. The composition of Embodiment 12, wherein the compound is of Formula I, or a tautomer or solvate thereof.

Embodiment 14. The composition of Embodiment 13, wherein the compound is of:

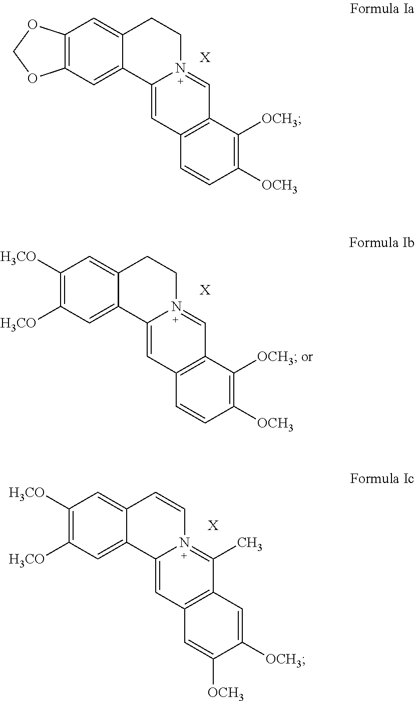

or a tautomer or solvate thereof.

Embodiment 15. The composition of Embodiment 12, wherein the compound is of Formula II, or a tautomer or solvate thereof.

Embodiment 16. The composition of Embodiment 15, wherein the compound is of:

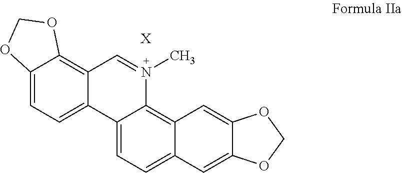

or a tautomer or solvate thereof.

Embodiment 17. The composition of any one of Embodiments 12-16, wherein X is a halide.

Embodiment 18. The composition of Embodiment 17, wherein X is chloride.

Embodiment 19. The composition of any one of Embodiments 12-18, wherein said nucleic acid formulation comprises lipid nanoparticles.

Embodiment 20. The composition of any one of Embodiments 12-18, wherein said nucleic acid formulation comprises liposomes.

Embodiment 21. The composition of any one of Embodiments 12-18, wherein said nucleic acid formulation comprises a lipoplex.

Embodiment 22. The composition of any one of Embodiments 19-21, wherein the nucleic acid is encapsulated within the lipid nanoparticles, liposomes, or lipoplex.

Embodiment 23. The composition of any one of Embodiments 12-22, wherein the nucleic acid is mRNA.

Embodiment 24. The composition of any one of Embodiments 12-23, wherein the compound has a purity of at least 70%, 80%, 90%, 95%, or 99%.

Embodiment 25. The composition of any one of Embodiments 12-24, wherein the compound contains fewer than 100 ppm of elemental metals.

Embodiment 26. The composition of any one of Embodiments 12-25, wherein the composition is formulated in an aqueous solution.

Embodiment 27. The composition of Embodiment 26, wherein the aqueous solution comprises lipid nanoparticles and wherein the nucleic acid is encapsulated in the lipid nanoparticles.

Embodiment 28. The composition of Embodiment 26 or 27, wherein the aqueous solution has a pH of or about 5 to 8, including pH of about 5, 5.5, 6, 6.5, 7, 7.5, or 8.

Embodiment 29. The composition of Embodiment 26 or 27, wherein the aqueous solution does not comprise NaCl.

Embodiment 30. The composition of Embodiment 26 or 27, wherein the aqueous solution comprises NaCl in a concentration of or about 150 mM.

Embodiment 31. The composition of any one of Embodiments 26-30, wherein the aqueous solution comprises a phosphate buffer, a tris buffer, an acetate buffer, a histidine buffer, or a citrate buffer.

Embodiment 32. The composition of any one of Embodiments 26-31, wherein the compound is present at a concentration of less than about 10 mM.

Embodiment 33. The composition of any one of Embodiments 26-32, wherein the compound is present at a concentration of or about 2 mM.

Embodiment 34. The composition of any one of Embodiments 26-32, wherein the compound is present at a concentration of or about 1 mM.

Embodiment 35. The composition of any one of Embodiments 26-32, wherein the compound is present at a concentration of or about 0.5 mM.

Embodiment 36. The composition of any one of Embodiments 12-25, wherein the nucleic acid is a lyophilized product.

Embodiment 37. The composition of Embodiment 36, wherein the lyophilized product comprises lipid nanoparticles wherein the nucleic acid is encapsulated in the lipid nanoparticles.

Embodiment 38. The composition of any one of Embodiments 1-2, and 4-37, further comprising a chelator.

Embodiment 39. The composition of Embodiment 38, wherein the composition is a solution comprising 1 μM-100 mM chelator.

Embodiment 40. The composition of Embodiment 39, wherein the solution comprises about 1 mM chelator.

Embodiment 41. The composition of any one of Embodiments 12-40, wherein the composition comprises a mRNA purity level of greater than 50% main peak mRNA purity after at least six months of storage at 2-8° C.

Embodiment 42. The composition of any one of Embodiments 12-40, wherein the composition comprises a mRNA purity level of greater than 50% main peak mRNA purity after at least twelve months of storage at 2-8° C.

Embodiment 43. The use of the composition of any one of Embodiments 1-2, and 4-42 for the treatment of a disease in a subject.

Embodiment 44. The use according to Embodiment 43, wherein the disease is caused by an infectious agent.

Embodiment 45. The use according to any one of Embodiments 43-44, wherein the disease is caused by or associated with a virus.

Embodiment 46. The use according to Embodiment 43, wherein the disease is caused by or associated with a malignant cell.

Embodiment 47. The use according to Embodiment 46, wherein the disease is cancer.

Embodiment 48. The composition of any one of Embodiments 1-2, and 4-42, or the use of any one of Embodiments 43-47, wherein microbial growth in the composition is inhibited by the compound.

Embodiment 49. The composition of any one of Embodiments 1-2, 4-42, and 48, or the use of any one of Embodiments 43-47, wherein the composition does not comprise phenol, m-cresol, or benzyl alcohol.

Embodiment 50. A method of formulating a nucleic acid comprising:
adding to a composition comprising a nucleic acid and a lipid, a compound of Formula I:

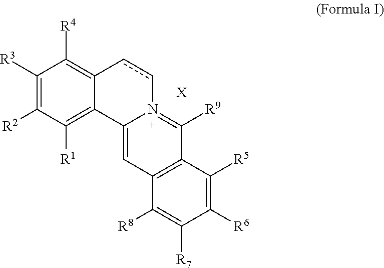

or a tautomer or solvate thereof, wherein:
[symbol] is a single bond or a double bond;
R¹ is H;
R² is OCH₃, or together with R³ is OCH₂O;
R³ is OCH₃, or together with R² is OCH₂O;
R⁴ is H;
R⁵ is H or OCH₃;
R⁶ is OCH₃;
R⁷ is H or OCH₃;
R⁸ is H;
R⁹ is H or CH₃; and
X is a pharmaceutically acceptable anion;
or a compound of Formula II:

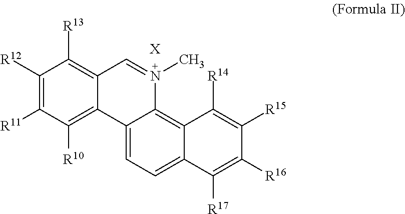

or a tautomer or solvate thereof, wherein:
R¹⁰ is H;
R¹¹ is H;
R¹² together with R¹³ is OCH₂O;
R¹⁴ is H;
R¹⁵ together with R¹⁶ is OCH₂O;
R¹⁷ is H; and
X is a pharmaceutically acceptable anion;
to obtain a formulated composition.

Embodiment 51. The method of Embodiment 50, wherein the compound is of Formula I, or a tautomer or solvate thereof.

Embodiment 52. The method of Embodiment 51, wherein the compound is of:

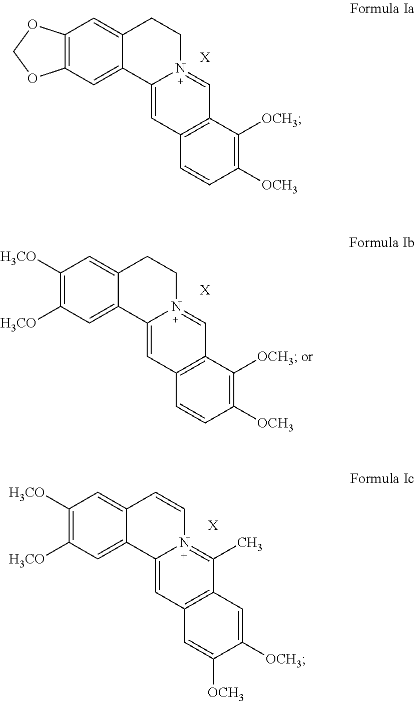

or a tautomer or solvate thereof.

Embodiment 53. The method of Embodiment 50, wherein the compound is of Formula II, or a tautomer or solvate thereof.

Embodiment 54. The method of Embodiment 53, wherein the compound is of:

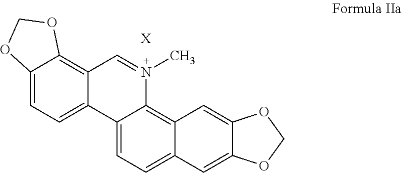

or a tautomer or solvate thereof.

Embodiment 55. The method of any one of Embodiments 50-54, wherein X is a halide.

Embodiment 56. The method of Embodiment 55, wherein X is chloride.

Embodiment 57. The method of any one of Embodiments 50-56, wherein the formulated composition comprises lipid nanoparticles.

Embodiment 58. The method of any one of Embodiments 50-56, wherein the formulated composition further comprises liposomes.

Embodiment 59. The method of any one of Embodiments 50-56, wherein the formulated composition further comprises a lipoplex.

Embodiment 60. The method of any one of Embodiments 50-59, wherein the nucleic acid is encapsulated in the lipid nanoparticles, liposomes, or lipoplex.

Embodiment 61. The method of Embodiment 50, further comprising subsequently removing the compound of Formula I or Formula II from the formulated composition.

Embodiment 62. The method of any one of Embodiments 50-61, wherein the compound has a purity of at least 70%, 80%, 90%, 95%, or 99%.

Embodiment 63. The method of any one of Embodiments 50-62, wherein the compound contains fewer than 100 ppm of elemental metals.

Embodiment 64. The method of any one of Embodiments 50-63, wherein the composition is formulated in an aqueous solution.

Embodiment 65. The method of Embodiment 64, wherein the aqueous solution comprises lipid nanoparticles and wherein a nucleic acid is encapsulated in the lipid nanoparticles.

Embodiment 66. The method of Embodiment 64 or 65, wherein the aqueous solution has a pH of or about 5 to 8, including pH of about 5, 5.5, 6, 6.5, 7, 7.5, or 8.

Embodiment 67. The method of any one of Embodiments 64-66, wherein the aqueous solution does not comprise NaCl.

Embodiment 68. The method of any one of Embodiments 64-66, wherein the aqueous solution comprises NaCl in a concentration of or about 150 mM.

Embodiment 69. The method of any one of Embodiments 64-68, wherein the aqueous solution comprises a phosphate buffer, a tris buffer, an acetate buffer, a histidine buffer, or a citrate buffer.

Embodiment 70. The method of any one of Embodiments 64-69, wherein the compound is present at a concentration of less than about 10 mM.

Embodiment 71. The method of any one of Embodiments 64-69, wherein the compound is present at a concentration of or about 2 mM.

Embodiment 72. The method of any one of Embodiments 64-69, wherein the compound is present at a concentration of or about 1 mM.

Embodiment 73. The method of any one of Embodiments 64-69, wherein the compound is present at a concentration of or about 0.5 mM.

Embodiment 74. The method of any one of Embodiments 50-73, wherein the composition is a lyophilized product.

Embodiment 75. The method of Embodiment 74, wherein the lyophilized product comprises lipid nanoparticles.

Embodiment 76. The method of Embodiment 75, wherein the lipid nanoparticles encapsulate a nucleic acid.

Embodiment 77. The method of any one of Embodiments 50-76, performed with shielding from light exposure.

Embodiment 78. The method of any one of Embodiments 50-77, performed under red light.

Embodiment 79. A pharmaceutically acceptable method of processing an mRNA-lipid nanoparticle for therapeutic injection, comprising adding a compound of Formula I or Formula II, or a tautomer or solvate thereof to a lipid nanoparticle, and subsequently adding an mRNA to the lipid nanoparticle-compound mixture.

Embodiment 80. A pharmaceutically acceptable method of conferring anti-microbial properties to an mRNA-lipid nanoparticle composition, comprising adding a compound of Formula I or Formula II, or a tautomer or solvate thereof to the mRNA-lipid nanoparticle composition.

Embodiment 81. A pharmaceutically acceptable method of processing an mRNA-lipid nanoparticle for therapeutic injection, comprising adding an mRNA to a lipid nanoparticle, and subsequently adding a compound of Formula I or Formula II, or a tautomer or solvate thereof to the lipid nanoparticle-mRNA mixture.

Embodiment 82. A pharmaceutically acceptable method of processing an mRNA-lipid nanoparticle for therapeutic injection, comprising combining an mRNA, a lipid nanoparticle, and a compound of Formula I or Formula II, or a tautomer or solvate thereof.

Embodiment 83. A composition comprising:
a lipid nanoparticle encapsulating a mRNA, wherein the composition comprises a mRNA purity level of greater than 50% main peak mRNA purity after at least thirty days of storage.

Embodiment 84. The composition of Embodiment 83, wherein the composition comprises a mRNA purity level of greater than 60% main peak mRNA purity after at least thirty days of storage.

Embodiment 85. The composition of Embodiment 83 or 84, wherein the composition comprises a mRNA purity level of greater than 70% main peak mRNA purity after at least thirty days of storage.

Embodiment 86. The composition of any one of Embodiments 83-85, wherein the composition comprises a mRNA purity level of greater than 80% main peak mRNA purity after at least thirty days of storage.

Embodiment 87. The composition of any one of Embodiments 83-86, wherein the composition comprises a mRNA purity level of greater than 90% main peak mRNA purity after at least thirty days of storage.

Embodiment 88. The composition of any one of Embodiments 83-87, wherein the composition comprises a mRNA purity level of greater than 50% main peak mRNA purity after at least six months of storage.

Embodiment 89. The composition of any one of Embodiments 83-88, wherein the composition comprises a mRNA purity level of greater than 50% main peak mRNA purity after at least twelve months of storage.

Embodiment 90. The composition of any one of Embodiments 83-89, wherein the storage is at room temperature.

Embodiment 91. The composition of any one of Embodiments 83-89, wherein the storage is at greater than room temperature.

Embodiment 92. The composition of any one of Embodiments 83-89, wherein the storage is at 4° C.

Embodiment 93. The composition of any one of Embodiments 83-92, wherein the composition comprises a compound of Formula I or Formula II, or a tautomer or solvate thereof.

Embodiment 94. The composition of Embodiment 93, wherein the compound of Formula I is of Formula Ia, Formula Ib, or Formula Ic.

Embodiment 95. The composition of Embodiment 93, wherein the compound is berberine, palmatine, coralyne, or sanguinarine, or a tautomer or solvate thereof.

Embodiment 96. A composition comprising:
a lipid nanoparticle encapsulating a mRNA, wherein the mRNA comprises intact mRNA and at least one RNA fragment, wherein the composition comprises less than 50% RNA fragments after at least thirty days of storage.

Embodiment 97. The composition of Embodiment 96, wherein the composition comprises less than 60% RNA fragments after at least thirty days of storage.

Embodiment 98. The composition of Embodiment 96 or 97, wherein the composition comprises less than 70% RNA fragments after at least thirty days of storage.

Embodiment 99. The composition of any one of Embodiments 96-98, wherein the composition comprises less than 80% RNA fragments after at least thirty days of storage.

Embodiment 100. The composition of any one of Embodiments 96-99, wherein the composition comprises less than 90% RNA fragments after at least thirty days of storage.

Embodiment 101. The composition of any one of Embodiments 96-100, wherein the composition comprises less than 95% RNA fragments after at least thirty days of storage.

Embodiment 102. The composition of any one of Embodiments 96-101, wherein the composition is stored for at least six months.

Embodiment 103. The composition of any one of Embodiments 96-102, wherein the storage is at room temperature.

Embodiment 104. The composition of any one of Embodiments 96-102, wherein the storage is at greater than room temperature.

Embodiment 105. The composition of any one of Embodiments 96-102, wherein the storage is at 4° C.

Embodiment 106. The composition of any one of Embodiments 96-105, wherein the composition comprises a compound of Formula I, or a tautomer or solvate thereof.

Embodiment 107. The composition of any one of Embodiments 96-105, wherein the composition comprises a compound of Formula II, or a tautomer or solvate thereof.

Embodiment 108. The composition of Embodiment 106 or 107, wherein the compound is berberine, palmatine, coralyne, or sanguinarine, or a tautomer or solvate thereof.

Embodiment 109. The composition of any one of Embodiments 96-108, wherein the lipid nanoparticle comprises a ratio of 20-60% amino lipids, 5-30% phospholipid, 10-55% structural lipid, and 0.5-15% PEG-modified lipid.

Embodiment 110. The composition of any one of Embodiments 96-109, wherein the lipid nanoparticle comprises a ratio of 20-60% amino lipids, 5-25% phospholipid, 25-55% structural lipid, and 0.5-15% PEG-modified lipid.

Embodiment 111. A method for producing a protein in a subject, comprising administering a composition of any one of Embodiments 12-42, 48, 49, or 83-110 to a subject, wherein the nucleic acid is an mRNA and wherein the mRNA encodes for the production of a protein in the subject.

Embodiment 112. A syringe or cartridge, comprising a composition of any one of Embodiments 12-42, 48, 49, or 83-110.

Embodiment 113. An infusion pump, comprising a composition of any one of Embodiments 12-42, 48, 49, or 83-110.

Embodiment 114. A syringe or cartridge, comprising multiple doses of a composition of any one of Embodiments 12-42, 48, 49, or 83-110.

Embodiment 115. A photoprotective container comprising the stabilized pharmaceutical composition, lipid nanoparticle, or composition of any one of Embodiments 1-42, 48, 49, or 83-110.

Embodiment 116. The photoprotective container of Embodiment 115, wherein the container prevents light from contacting the stabilized pharmaceutical composition, lipid nanoparticle, or composition.

Embodiment 117. The photoprotective container of Embodiment 116, wherein the container comprises a film, foil, or coating.

Embodiment 118. The method of any one of Embodiments 50-82 and 111, performed in a photoprotective container, performed in the absence of sunlight, room light, UV light, and/or fluorescent light, and/or performed under red light.

Embodiment 119. The syringe or cartridge, or infusion pump, of any one of Embodiments 112-114, wherein the syringe or cartridge, or infusion pump is a photoprotective container.

Embodiment 120. The photoprotective container of any one of Embodiments 115-117, the method of Embodiment 118, or the syringe, cartridge, or infusion pump of Embodiment 119, wherein the photoprotective container reduces the amount of a reactive species (e.g, a decomposition product, such as an aldehyde) in the pharmaceutical composition, lipid nanoparticle, or composition.

### EXAMPLES

### Example 1

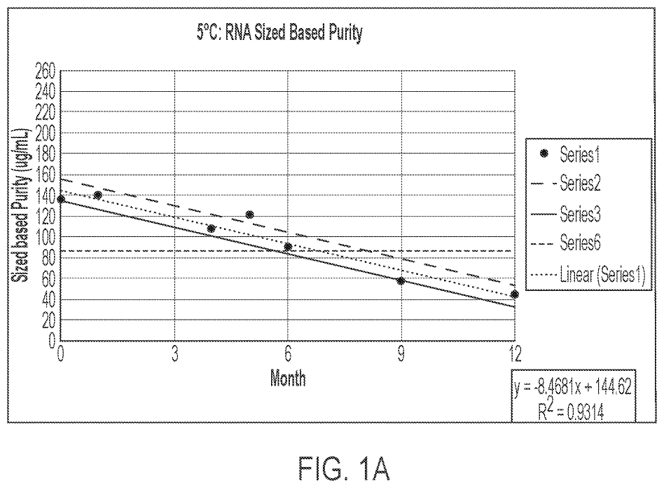

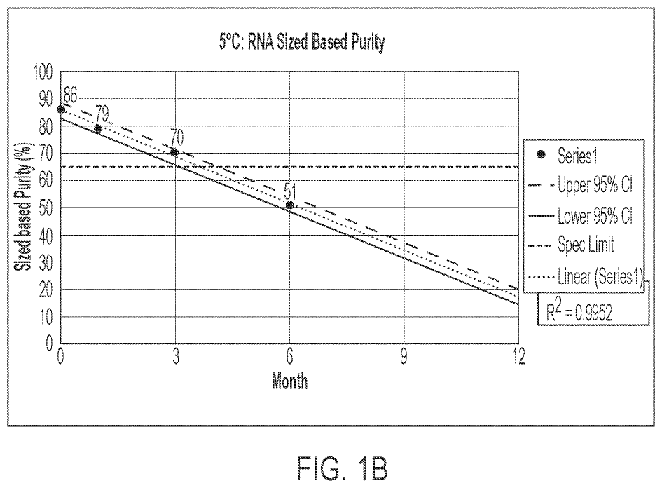

This example describes the instability of RNA in lipid nanoparticle formulations when stored as a refrigerated liquid. One of the most formidable barriers to translating the concept of using messenger RNA as a pharmaceutical agent is the inherent instability of the mRNA molecule. RNA is highly susceptible to chemical and enzymatic cleavage as well as adduct formation, which causes a loss of translational potency. Lipid nanoparticle (LNP) formulations of mRNA thus undergo rapid loss of purity when stored as a refrigerated liquid. FIGS. 1A and 1B demonstrate that the shelf life of LNP-mRNA formulations falls below this cutoff. Consequently, most mRNA formulations must be stored frozen at −20° C. or −80° C. Although these storage conditions may be viable in the case of rare disease treatment or highly specialized indications, they are far from ideal. Additionally, refrigerated liquid products are preferred over −80° C. products as they are more patient-friendly for widespread use. The ability to formulate mRNA drug products in refrigerated liquid compositions would facilitate widespread use of mRNA drugs, such as for vaccine products, which are typically provided as shelf-stable injectables requiring no special reconstitution or storage conditions.

mRNA Stabilization with Berberine

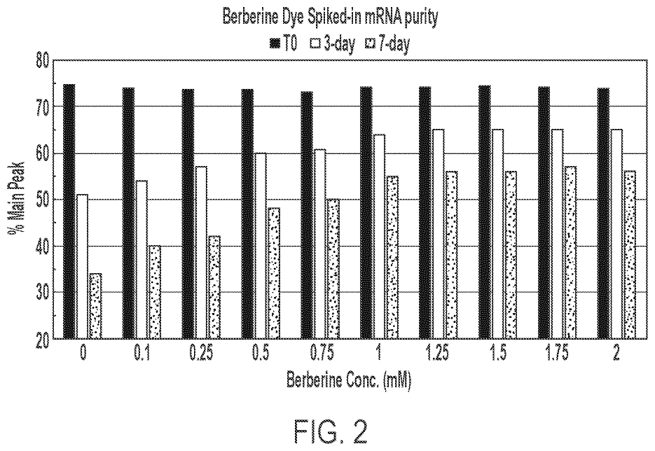

Berberine USP was combined with an LNP containing mRNA (0.2 mg/ml in buffer) in amounts corresponding to the concentrations reported in FIG. 2. Main peak purity was measured by RP-HPLC at T0, 3-day, and 7-day. See, FIG. 2.

mRNA Stabilization with Palmatine

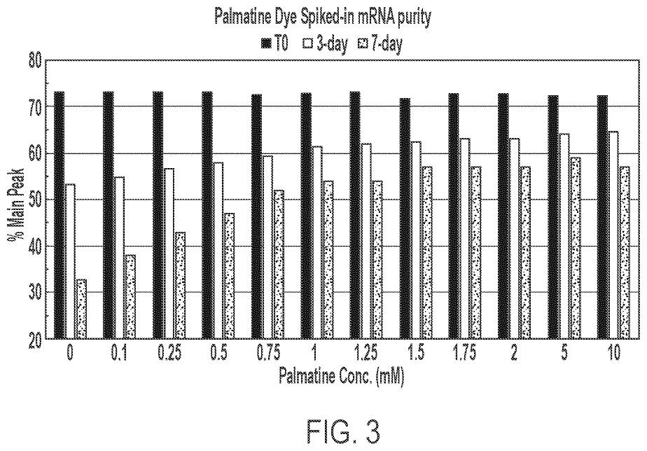

Palmatine USP was combined with an LNP containing mRNA (0.2 mg/mL in buffer) in amounts corresponding to the concentrations reported in FIG. 3. Main peak purity was measured by RP-HPLC at T0, 3-day, and 7-day. See, FIG. 3.

mRNA Stabilization with Coralyne Versus Palmatine

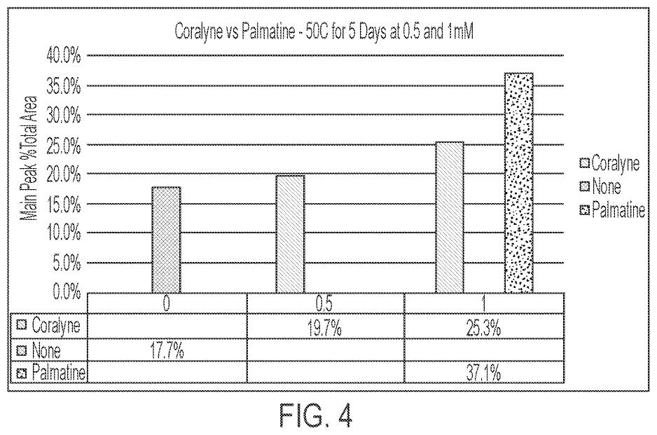

Coralyne USP and Palmatine USP were combined with buffered formulations of mRNA in amounts corresponding to the concentrations reported in FIG. 4. After storage for 5 days at 50° C., main peak purity was measured by RP-HPLC. See, FIG. 4.

Each of berberine, palmatine, and coralyne showed a stabilizing effect on mRNA.

### Example 2

A bacterial reverse mutation assay is run to evaluate the mutagenic potential of the palmatine and berberine by measuring their ability to induce reverse mutations at selected loci of several strains of Salmonella typhimurium and at the tryptophan locus of Escherichia coli WP2 uvrA in the presence and absence of an exogenous metabolic activation system. The tester strains include the S. typhimurium histidine auxotrophs TA98, TA100, TA1535 and TA97a as described by Ames et al. (1975) and the E. coli tester strain WP2 uvrA as described by Green and Muriel (1976).

A test system (e.g., plate) comprising a tester strain is exposed to a vehicle alone (e.g., DMSO) or various concentrations of palmatine or berberine, both in the presence and absence of liver homogenate S9. The test system is incubated for an incubation period at 2-8° C. The condition of the bacterial lawn is then evaluated for evidence of toxicity and precipitate. Toxicity is scored relative to a vehicle control; toxicity is evaluated as a decrease in the number of revertant colonies and/or a thinning or disappearance of the bacterial lawn background. Precipitation is evaluated by visual examination without magnification.

For strain TA1535, data sets are judged positive if the increase in mean revertants at the peak of the dose response is equal to or greater than 3.0-times the mean vehicle control value and above the corresponding acceptable vehicle control range with a minimum of 6 revertants. For strains TA98, TA100, TA97a and WP2 uvrA, data sets are judged positive if the increase in mean revertants at the peak of the dose response is equal to or greater than 2.0-times the mean vehicle control value and above the corresponding acceptable vehicle control range with a minimum of 6 revertants. An equivocal response is an increase in a revertant count that is greater than the acceptable vehicle control range but lacks a dose response or does not achieve the respective fold increase threshold cited. A response is evaluated as negative, if it is neither positive nor equivocal.

### Example 3

Experiments were performed to (i) evaluate immunogenicity of mRNA LNPs containing palmatine or berberine at varying concentrations as compared to non-stabilized control mRNA LNPs.

BALB/c mice (N=210) are injected with a mRNA LNP stabilized with varying concentrations of palmatine or berberine (e.g., 0, 0.5 mM, 0.75 mM, 1 mM, 2 mM, 5 mM, or 10 mM). Various dose levels (e.g., 2.5 ug/mg; 1.25 ug/mg; or 0.63 ug/mg) are injected into mice intramuscularly on day 1. On day 21, immunogenicity is assessed via serum antibody titers against protein encoded by the mRNA. The mice are given a booster injection on day 22, and then immunogenicity is again assessed on day 36.

Three month, 25° C. stability samples from mRNA LNPs containing palmatine at varying concentrations, and non-stabilized mRNA LNPs (no palmatine) were tested in vivo as continued immunogenicity monitoring on stability. The results demonstrate that the non-stabilized samples (25° C.) are beginning to exhibit non-equivalent antibody titers compared to the −70° C. non-stabilized control samples. The drop in antibody titers is correlated to a significant drop in mRNA purity. The stabilized samples all maintain higher mRNA purity than the non-stabilized after 3 MTHs at 25° C. This higher purity is correlated with more similar antibody titers to the −70° C. control arm.

### Example 4

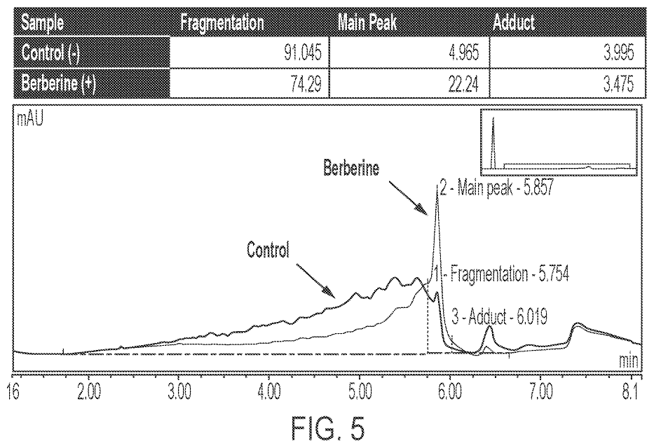

HPLC analyses were performed to evaluate the purity of mRNA stressed at elevated temperature (40° C.) for 3 weeks. In the absence of stabilizer, less than 5% of the mRNA (main peak 5.857 min) remained intact. By contrast, in the presence of ˜1 mM berberine, over 22% of the mRNA (main peak 5.857 min) remained intact. These data show that berberine conferred a significant degree of stability with respect to loss of mRNA purity at elevated temperature. See, FIG. 5.

### Example 5

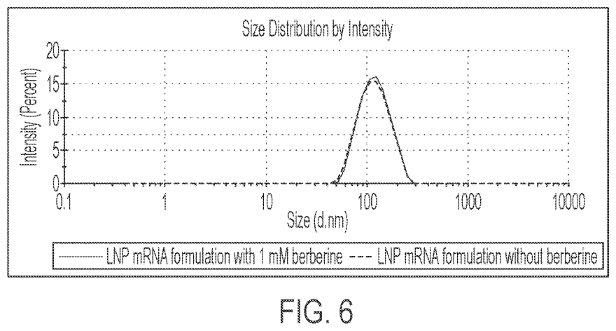

Dynamic light scattering is sensitive to perturbation to the physical integrity of mRNA LNPs. mRNA LNPs were formulated with 1 mM berberine or without berberine and then exposed to 40° C. temperature stress for 1 week. As a control, mRNA LNPs were formulated without berberine. The size distribution of the mRNA LNPs was then analyzed by dynamic light scattering. It was apparent from the intensity distributions that the presence of berberine had no effect on the hydrodynamic characteristics of the lipid nanoparticle. The mRNA LNP retains good physical stability in the presence of berberine, suggesting good physical compatibility of berberine for pharmaceutical formulation. See, FIG. 6.

### Example 6

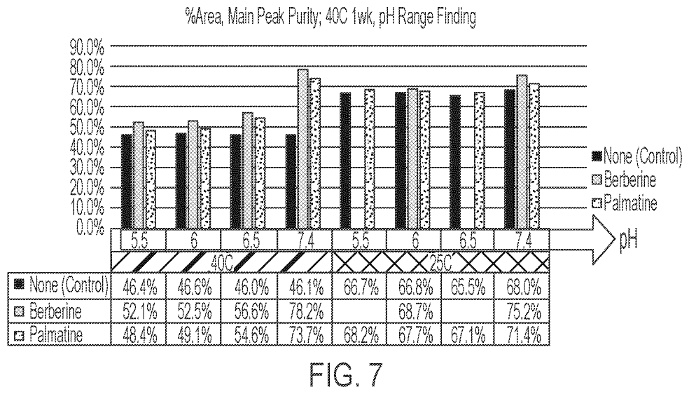

The stability of mRNA LNP formulations was evaluated as a function of pH and temperature in the presence and absence of berberine and palmatine. 1 mM excipients were added in pH conditions 5.5 to 7.4 to evaluate pH impact on mRNA stabilization in mRNA LNPs. For pH between 5.5-6.5, 7 mM citrate and 8% sucrose buffer was used. For pH of 7.4, tris/sucrose buffer was used. The LNP formulations were incubated at 0.8 mg/mL for 1 week at 40° C. or 2 weeks at 25° C., and then main peak purity was measured. Incubation for 1 week at 40° C. or 2 weeks at 25° C. show similar trends. Berberine and palmatine both show strong stabilizing effects across a wide pH range of LNP formulations, including at pH of 7.4. See, FIG. 7.

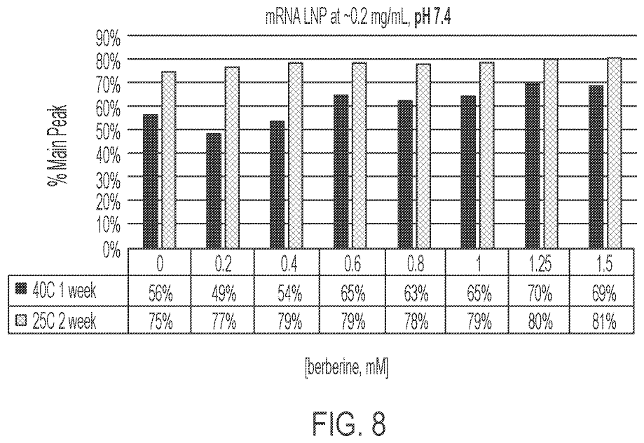

The stability of mRNA LNP formulations at pH of 7.4 was then further evaluated as a function of berberine concentration. A series of mRNA LNPs were formulated with various levels of berberine (0, 0.2, 0.4, 0.6, 0.8, 1.0, 1.25, 1.5 mM) and stressed in 7.4 pH tris/sucrose buffer at ˜0.2 mg/mL for either 1 week at 40° C. or 2 weeks at 25° C., and then main peak purity was measured. The data show that the stabilizing effect of berberine occurs in a concentration-dependent manner, under both 25° C. and 40° C. stresses. In this study, maximal effect occurs in the 1-1.25 mM berberine range. See, FIG. 8.

### Example 7

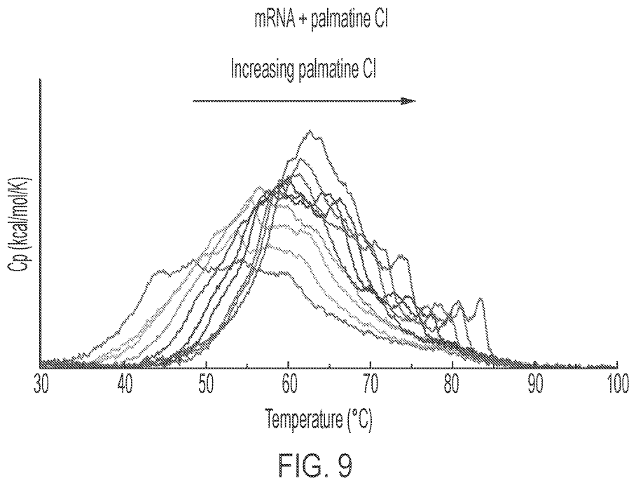

With reference to FIG. 9, differential scanning microcalorimetry thermograms were obtained of lipid nanoparticles encapsulating mRNA and containing increasing concentrations of palmatine chloride (left-most trace—free mRNA, right-most trace—500 μM palmatine). The experiments were performed in 20 mM Tris, 8% sucrose buffer. The data shows that palmatine binding leads to increased thermal stability of mRNA.

### Example 8

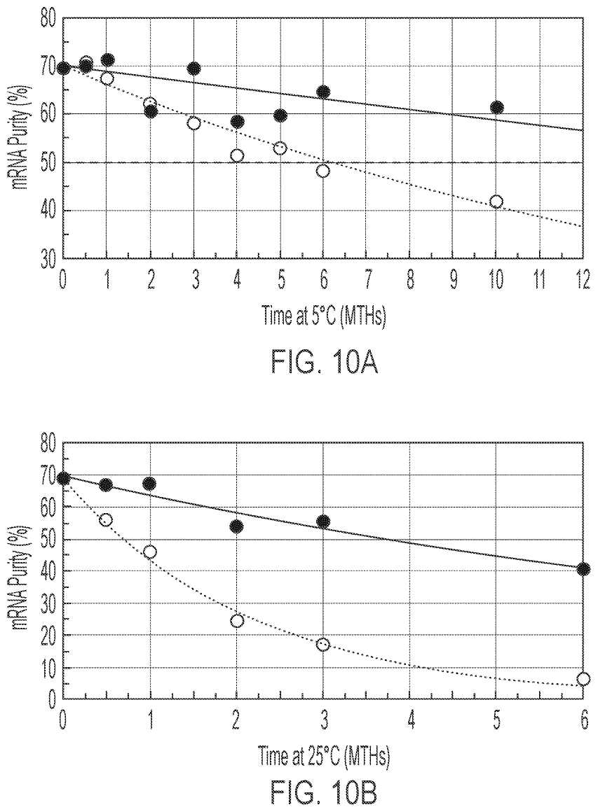

With reference to FIGS. 10A and 10B, studies were performed to determine the long-term effect of palmatine chloride on thermal stability of mRNA. Aliquots of mRNA solution were mixed with 2 mM palmatine chloride and were incubated for the indicated periods of time at 5° C. (FIG. 10A) and 25° C. (FIG. 10B). The percentage of remaining intact mRNA was quantified using chromatography. The dashed line traces represent mRNA-only samples. The solid line traces represent mRNA samples containing palmatine chloride. The data shows that mRNA thermal stability is significantly improved in the presence of palmatine chloride.

### Example 9

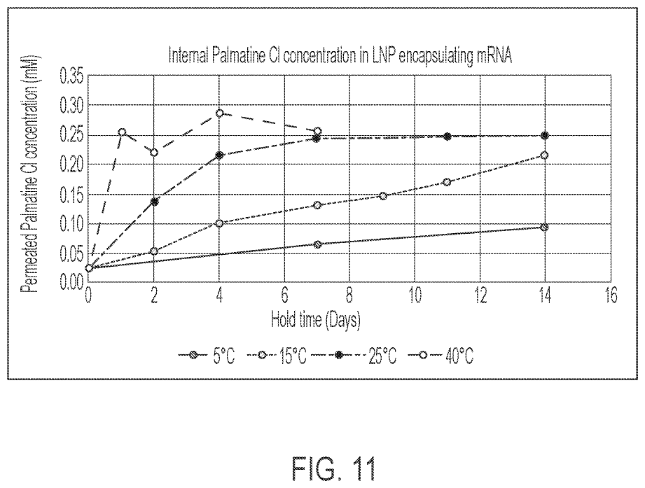

With reference to FIG. 11, studies were performed to determine the effect of time and temperature on diffusion (permeation) of palmatine chloride into lipid nanoparticles encapsulating mRNA. The samples were prepared in 20 mM Tris, 8% sucrose. The lipid nanoparticles encapsulating mRNA were incubated in 2 mM palmatine for 1 to 14 days. Free palmatine was separated from the lipid nanoparticles by gel filtration on 40 kD Zeba cartridges, whereas the concentration of lipid nanoparticle-permeated palmatine was determined by reverse phase chromatography using a Luna C18 column. The data shows an increased rate of permeation, and increased permeated palmatine concentration, as a function of temperature.

### Example 10

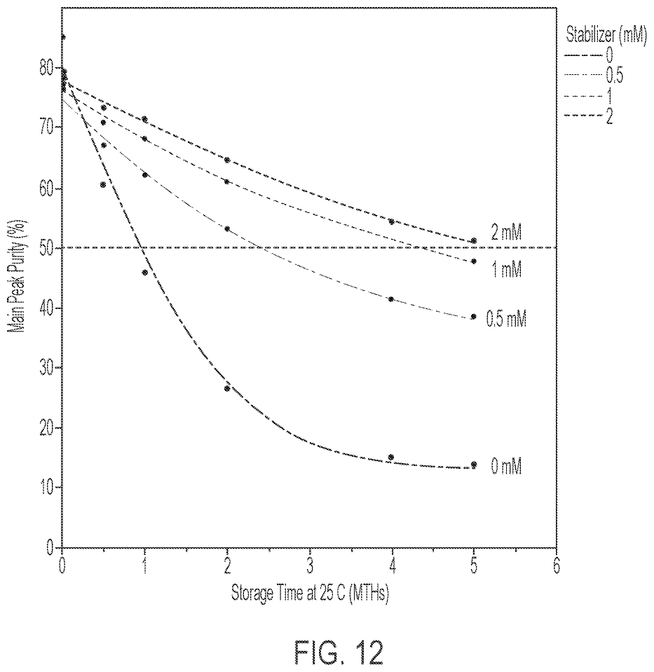

With reference to FIG. 12, studies were performed to determine the stability of lipid nanoparticles encapsulating mRNA containing the chelator DTPA and varying concentrations of palmatine chloride. 0.20 mg/ml samples of lipid nanoparticles encapsulating mRNA and 1 mM DTPA were incubated with palmatine chloride (0 mM, 0.5 mM, 1 mM, or 2 mM). Measurements of main peak (mRNA) purity were taken at time points up to 5 months. The data shows that mRNA stability is significantly improved in the presence of palmatine chloride.

### EQUIVALENTS

While several embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments are provided that are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying.” “having,” “containing,” “involving.” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A stabilized pharmaceutical composition comprising a lipid nanoparticle (LNP) encapsulated nucleic acid and a stabilizing compound that reduces adduct formation between the nucleic acid and a lipid of the LNP and/or nucleic acid degradation in the composition.

2. A stabilized pharmaceutical composition comprising a lipid nanoparticle (LNP) encapsulated nucleic acid and a stabilizing compound that physically interacts with the nucleic acid.

3. A lipid nanoparticle (LNP) comprising a nucleic acid and a stabilizing compound that physically interacts with the nucleic acid.

4. A stabilized pharmaceutical composition comprising a lipid nanoparticle (LNP) encapsulated nucleic acid and an intercalating stabilizer that binds to the nucleic acid with a micromolar dissociation constant.

5. A stabilized pharmaceutical composition comprising a lipid nanoparticle (LNP) comprising mRNA and a stabilizing compound reversibly bound to double stranded regions of the mRNA.

6. A stabilized pharmaceutical composition comprising a lipid nanoparticle (LNP) encapsulated nucleic acid and an intercalating stabilizer, wherein the composition does not contain an excipient stabilizer that functions via a chemical reactivity mechanism.

7. A stabilized pharmaceutical composition comprising a lipid nanoparticle (LNP) encapsulated mRNA reversibly bound to an intercalating small molecule that is cationic, soluble in aqueous solution, and capable of permeating a lipid nanoparticle.

8. A stabilized pharmaceutical composition comprising a lipid nanoparticle (LNP) encapsulated nucleic acid bound to a stabilizing compound, wherein the composition is substantially free of unbound stabilizing compound.

9. A stabilized pharmaceutical composition comprising lipid nanoparticles comprising a nucleic acid and a stabilizing compound, wherein substantially all of the stabilizing compound is located in or on the lipid nanoparticles.

10. A stabilized pharmaceutical composition comprising a lipid nanoparticle (LNP) encapsulated mRNA and an amount of an isoquinoline alkaloid, or a derivative thereof, effective to stabilize the composition.

11. The stabilized pharmaceutical composition of any of the preceding claims, wherein the LNP comprises a molar ratio of about 20-60% ionizable cationic lipid:about 5-25% non-cationic lipid:about 25-55% sterol; and about 0.5-15% PEG-modified lipid.

12. A stabilized pharmaceutical composition comprising:
a nucleic acid formulation comprising a nucleic acid and a lipid, and a compound of Formula I: 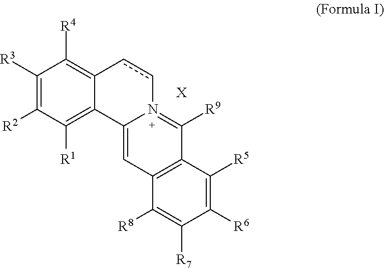
or a tautomer or solvate thereof, wherein: [symbol]
is a single bond or a double bond;
R¹ is H;
R² is OCH₃, or together with R³ is OCH₂O;
R³ is OCH₃, or together with R² is OCH₂O;
R⁴ is H;
R⁵ is H or OCH₃;
R⁶ is OCH₃;
R⁷ is H or OCH₃;
R⁸ is H;
R⁹ is H or CH₃; and
X is a pharmaceutically acceptable anion;
or a compound of Formula II: 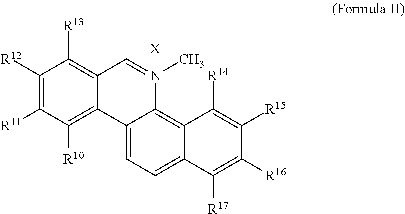
or a tautomer or solvate thereof, wherein:
R¹⁰ is H;
R¹¹ is H;
R¹² together with R¹³ is OCH₂O;
R¹⁴ is H;
R¹⁵ together with R¹⁶ is OCH₂O;
R¹⁷ is H; and
X is a pharmaceutically acceptable anion.

13. The composition of claim 12, wherein the compound is of Formula I, or a tautomer or solvate thereof.

14. The composition of claim 13, wherein the compound is of: 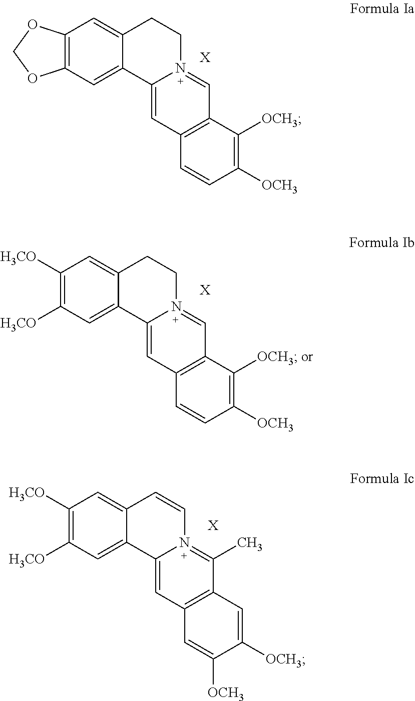
or a tautomer or solvate thereof.

15. The composition of claim 12, wherein the compound is of Formula II, or a tautomer or solvate thereof.

16. The composition of claim 15, wherein the compound is of: 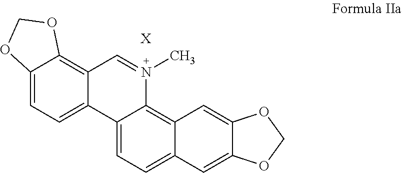
or a tautomer or solvate thereof.

17. The composition of claim 12, wherein X is a halide.

18. The composition of claim 17, wherein X is chloride.

19. The composition of claim 12, wherein said nucleic acid formulation comprises lipid nanoparticles.

20. The composition of claim 12, wherein said nucleic acid formulation comprises liposomes.

21. The composition of claim 12, wherein said nucleic acid formulation comprises a lipoplex.

22. The composition of any one of claims 19-21, wherein the nucleic acid is encapsulated within the lipid nanoparticles, liposomes, or lipoplex.

23. The composition of claim 12, wherein the nucleic acid is mRNA.

24. The composition of claim 12, wherein the compound has a purity of at least 70%, 80%, 90%, 95%, or 99%.

25. The composition of claim 12, wherein the compound contains fewer than 100 ppm of elemental metals.

26. The composition of claim 12, wherein the composition is formulated in an aqueous solution.

27. The composition of claim 26, wherein the aqueous solution comprises lipid nanoparticles and wherein the nucleic acid is encapsulated in the lipid nanoparticles.

28. The composition of claim 26 or 27, wherein the aqueous solution has a pH of or about 5 to 8, including pH of about 5, 5.5, 6, 6.5, 7, 7.5, or 8.

29. The composition of claim 26 or 27, wherein the aqueous solution does not comprise NaCl.

30. The composition of claim 26 or 27, wherein the aqueous solution comprises NaCl in a concentration of or about 150 mM.

31. The composition of claim 26, wherein the aqueous solution comprises a phosphate buffer, a tris buffer, an acetate buffer, a histidine buffer, or a citrate buffer.

32. The composition of claim 26, wherein the compound is present at a concentration of less than about 10 mM.

33. The composition of claim 26, wherein the compound is present at a concentration of or about 2 mM.

34. The composition of claim 26, wherein the compound is present at a concentration of or about 1 mM.

35. The composition of claim 26, wherein the compound is present at a concentration of or about 0.5 mM.

36. The composition of claim 12, wherein the nucleic acid is a lyophilized product.

37. The composition of claim 36, wherein the lyophilized product comprises lipid nanoparticles wherein the nucleic acid is encapsulated in the lipid nanoparticles.

38. The composition of claim 12, further comprising a chelator.

39. The composition of claim 38, wherein the composition is a solution comprising 1 μM-100 mM chelator.

40. The composition of claim 39, wherein the solution comprises about 1 mM chelator.

41. The composition of claim 12, wherein the composition comprises a mRNA purity level of greater than 50% main peak mRNA purity after at least six months of storage at 2-8° C.

42. The composition of claim 12, wherein the composition comprises a mRNA purity level of greater than 50% main peak mRNA purity after at least twelve months of storage at 2-8° C.

43. The use of the composition of claim 12 for the treatment of a disease in a subject.

44. The use according to claim 43, wherein the disease is caused by an infectious agent.

45. The use according to any one of claims 43-44, wherein the disease is caused by or associated with a virus.

46. The use according to claim 43, wherein the disease is caused by or associated with a malignant cell.

47. The use according to claim 46, wherein the disease is cancer.

48. The composition of claim 12, or the use of claim 43, wherein microbial growth in the composition is inhibited by the compound.

49. The composition of claim 12, or the use of claim 43, wherein the composition does not comprise phenol, m-cresol, or benzyl alcohol.

50. A method of formulating a nucleic acid comprising:
adding to a composition comprising a nucleic acid and a lipid, a compound of Formula I: 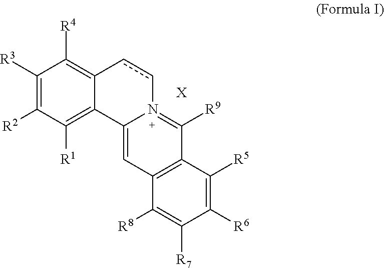
or a tautomer or solvate thereof, wherein: [symbol]
is a single bond or a double bond;
R¹ is H;
R² is OCH₃, or together with R³ is OCH₂O;
R³ is OCH₃, or together with R² is OCH₂O;
R⁴ is H;
R⁵ is H or OCH₃;
R⁶ is OCH₃;
R⁷ is H or OCH₃;
R⁸ is H;
R⁹ is H or CH₃; and
X is a pharmaceutically acceptable anion;
or a compound of Formula II: 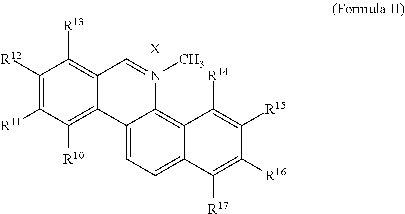
or a tautomer or solvate thereof, wherein:
R¹⁰ is H;
R¹¹ is H;
R¹² together with R¹³ is OCH₂O;
R¹⁴ is H;
R¹⁵ together with R¹⁶ is OCH₂O;
R¹⁷ is H; and
X is a pharmaceutically acceptable anion;
to obtain a formulated composition.

51. The method of claim 50, wherein the compound is of Formula I, or a tautomer or solvate thereof.

52. The method of claim 51, wherein the compound is of: 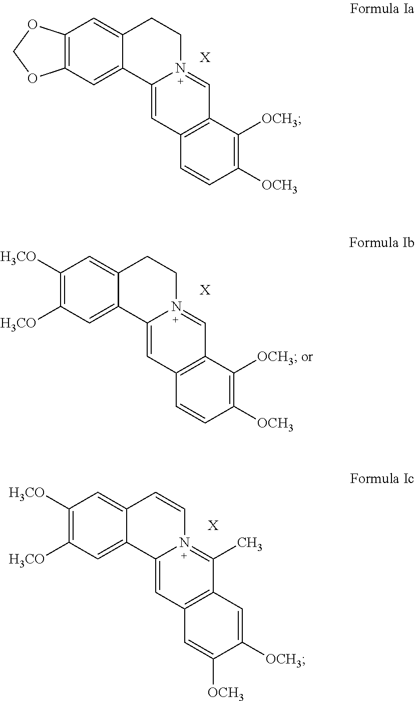
or a tautomer or solvate thereof.

53. The method of claim 50, wherein the compound is of Formula II, or a tautomer or solvate thereof.

54. The method of claim 53, wherein the compound is of: 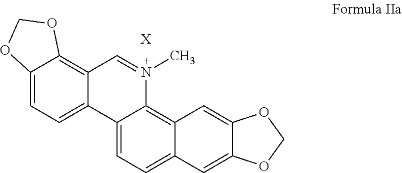
or a tautomer or solvate thereof.

55. The method of any one of claims 50-54, wherein X is a halide.

56. The method of claim 55, wherein X is chloride.

57. The method of claim 50, wherein the formulated composition comprises lipid nanoparticles.

58. The method of claim 50, wherein the formulated composition further comprises liposomes.

59. The method of claim 50, wherein the formulated composition further comprises a lipoplex.

60. The method of claim 50, wherein the nucleic acid is encapsulated in the lipid nanoparticles, liposomes, or lipoplex.

61. The method of claim 50, further comprising subsequently removing the compound of Formula I or Formula II from the formulated composition.

62. The method of claim 50, wherein the compound has a purity of at least 70%, 80%, 90%, 95%, or 99%.

63. The method of claim 50, wherein the compound contains fewer than 100 ppm of elemental metals.

64. The method of claim 50, wherein the composition is formulated in an aqueous solution.

65. The method of claim 64, wherein the aqueous solution comprises lipid nanoparticles and wherein a nucleic acid is encapsulated in the lipid nanoparticles.

66. The method of claim 64 or 65, wherein the aqueous solution has a pH of or about 5 to 8, including pH of about 5, 5.5, 6, 6.5, 7, 7.5, or 8.

67. The method of claim 64, wherein the aqueous solution does not comprise NaCl.

68. The method of claim 64, wherein the aqueous solution comprises NaCl in a concentration of or about 150 mM.

69. The method of claim 64, wherein the aqueous solution comprises a phosphate buffer, a tris buffer, an acetate buffer, a histidine buffer, or a citrate buffer.

70. The method of claim 64, wherein the compound is present at a concentration of less than about 10 mM.

71. The method of claim 64, wherein the compound is present at a concentration of or about 2 mM.

72. The method of claim 64, wherein the compound is present at a concentration of or about 1 mM.

73. The method of claim 64, wherein the compound is present at a concentration of or about 0.5 mM.

74. The method of claim 50, wherein the composition is a lyophilized product.

75. The method of claim 74, wherein the lyophilized product comprises lipid nanoparticles.

76. The method of claim 75, wherein the lipid nanoparticles encapsulate a nucleic acid.

77. The method of claim 50, performed with shielding from light exposure.

78. The method of claim 50, performed under red light.

79. A pharmaceutically acceptable method of processing an mRNA-lipid nanoparticle for therapeutic injection, comprising adding a compound of Formula I or Formula II, or a tautomer or solvate thereof to a lipid nanoparticle, and subsequently adding an mRNA to the lipid nanoparticle-compound mixture.

80. A pharmaceutically acceptable method of conferring anti-microbial properties to an mRNA-lipid nanoparticle composition, comprising adding a compound of Formula I or Formula II, or a tautomer or solvate thereof to the mRNA-lipid nanoparticle composition.

81. A pharmaceutically acceptable method of processing an mRNA-lipid nanoparticle for therapeutic injection, comprising adding an mRNA to a lipid nanoparticle, and subsequently adding a compound of Formula I or Formula II, or a tautomer or solvate thereof to the lipid nanoparticle-mRNA mixture.

82. A pharmaceutically acceptable method of processing an mRNA-lipid nanoparticle for therapeutic injection, comprising combining an mRNA, a lipid nanoparticle, and a compound of Formula I or Formula II, or a tautomer or solvate thereof.

83. A composition comprising:
a lipid nanoparticle encapsulating a mRNA, wherein the composition comprises a mRNA purity level of greater than 50% main peak mRNA purity after at least thirty days of storage.

84. The composition of claim 83, wherein the composition comprises a mRNA purity level of greater than 60% main peak mRNA purity after at least thirty days of storage.

85. The composition of claim 83 or 84, wherein the composition comprises a mRNA purity level of greater than 70% main peak mRNA purity after at least thirty days of storage.

86. The composition of claim 83, wherein the composition comprises a mRNA purity level of greater than 80% main peak mRNA purity after at least thirty days of storage.

87. The composition of claim 83, wherein the composition comprises a mRNA purity level of greater than 90% main peak mRNA purity after at least thirty days of storage.

88. The composition of claim 83, wherein the composition comprises a mRNA purity level of greater than 50% main peak mRNA purity after at least six months of storage.

89. The composition of claim 83, wherein the composition comprises a mRNA purity level of greater than 50% main peak mRNA purity after at least twelve months of storage.

90. The composition of claim 83, wherein the storage is at room temperature.

91. The composition of claim 83, wherein the storage is at greater than room temperature.

92. The composition of claim 83, wherein the storage is at 4° C.

93. The composition of claim 83, wherein the composition comprises a compound of Formula I or Formula II, or a tautomer or solvate thereof.

94. The composition of claim 93, wherein the compound of Formula I is of Formula Ia, Formula Ib, or Formula Ic.

95. The composition of claim 93, wherein the compound is berberine, palmatine, coralyne, or sanguinarine, or a tautomer or solvate thereof.

96. A composition comprising:
a lipid nanoparticle encapsulating a mRNA, wherein the mRNA comprises intact mRNA and at least one RNA fragment, wherein the composition comprises less than 50% RNA fragments after at least thirty days of storage.

97. The composition of claim 96, wherein the composition comprises less than 60% RNA fragments after at least thirty days of storage.

98. The composition of claim 96 or 97, wherein the composition comprises less than 70% RNA fragments after at least thirty days of storage.

99. The composition of claim 96, wherein the composition comprises less than 80% RNA fragments after at least thirty days of storage.

100. The composition of claim 96, wherein the composition comprises less than 90% RNA fragments after at least thirty days of storage.

101. The composition of claim 96, wherein the composition comprises less than 95% RNA fragments after at least thirty days of storage.

102. The composition of claim 96, wherein the composition is stored for at least six months.

103. The composition of claim 96, wherein the storage is at room temperature.

104. The composition of claim 96, wherein the storage is at greater than room temperature.

105. The composition of claim 96, wherein the storage is at 4° C.

106. The composition of claim 96, wherein the composition comprises a compound of Formula I, or a tautomer or solvate thereof.

107. The composition of claim 96, wherein the composition comprises a compound of Formula II, or a tautomer or solvate thereof.

108. The composition of claim 106 or 107, wherein the compound is berberine, palmatine, coralyne, or sanguinarine, or a tautomer or solvate thereof.

109. The composition of claim 96, wherein the lipid nanoparticle comprises a ratio of 20-60% amino lipids, 5-30% phospholipid, 10-55% structural lipid, and 0.5-15% PEG-modified lipid.

110. The composition of claim 96, wherein the lipid nanoparticle comprises a ratio of 20-60% amino lipids, 5-25% phospholipid, 25-55% structural lipid, and 0.5-15% PEG-modified lipid.

111. A method for producing a protein in a subject, comprising
administering a composition of claim 12 to a subject, wherein the nucleic acid is an mRNA and wherein the mRNA encodes for the production of a protein in the subject.

112. A syringe or cartridge, comprising a composition of claim 12.

113. An infusion pump, comprising a composition of claim 12.

114. A syringe or cartridge, comprising multiple doses of a composition of claim 12.

115. A photoprotective container comprising the composition of claim 12.

116. The photoprotective container of claim 115, wherein the container prevents light from contacting the stabilized pharmaceutical composition, lipid nanoparticle, or composition.

117. The photoprotective container of claim 116, wherein the container comprises a film, foil, or coating.

118. The method of claim 50, performed in a photoprotective container, performed in the absence of sunlight, room light, UV light, and/or fluorescent light, and/or performed under red light.

119. The syringe or cartridge of claim 112 or 114, wherein the syringe or cartridge is a photoprotective container.

120. The photoprotective container of any one of claims 115-117, the method of claim 118, or the syringe or cartridge of claim 119, wherein the photoprotective container reduces the amount of a reactive decomposition product in the composition.
